(19) United States
(12) Patent Application Publication
(10) Pub. No.: US 2023/0383011 A1
(43) Pub. Date: Nov. 30, 2023

(54) SITE SPECIFIC NOTCH-ACTIVATING MOLECULE AND USES THEREOF

(71) Applicant: Chugai Seiyaku Kabushiki Kaisha, Tokyo (JP)

(72) Inventors: Zhen Ning Adrian WEE, Singapore (SG); Yoshinao RUIKE, Kanagawa (JP); Hiroyasu MURAMATSU, Shizuoka (JP); Tomoyuki IGAWA, Tokyo (JP)

(21) Appl. No.: 18/010,611

(22) PCT Filed: Jun. 30, 2021
(86) PCT No.: PCT/JP2021/024655

§ 371 Date: Dec. 15, 2022

(30) Foreign Application Priority Data
Jun. 30, 2020 (JP) 2020-112553

(51) Int. Cl. C07K 16/46; C12N 15/63; A61P 35/00

(52) U.S. Cl. CPC C07K 16/468; C12N 15/63; A61P 35/00; C07K 2317/31; C07K 2317/52

(57) ABSTRACT

The present invention relates to Notch receptor-targeting multispecific antigen-binding molecules, uses thereof, etc. The present invention provides multispecific antigen-binding molecules that comprise a first antigen-binding moiety which specifically binds to a Notch receptor on a first target cell, and a second antigen-binding moiety which specifically binds to an anchor antigen on a second target cell. Furthermore, the inventors demonstrate anchorage-dependent Notch signalling activation by the multispecific antigen-binding molecules of the invention.

### TECHNICAL FIELD

The present disclosure relates to Notch receptor targeting multispecific antigen-binding molecules, uses thereof, and such.

### BACKGROUND ART

### Introduction on Notch Receptor Family and Notch Ligands

The Notch signalling is a highly conserved process which is essential to a diverse spectrum of cellular systems, and its deregulation has been implicated in a vast number of developmental disorders and malignancies. The Notch family of transmembrane receptors comprises of four protein paralogs (Notch1-4) in humans and mice, with largely non-redundant functions. Prior to plasma membrane localization, the Notch receptor is post-translationally cleaved at S1 site. This cleavage occurs inside the trans-Golgi network mediated by a furin-like protease (NPL 1). The two polypeptides that form the mature membrane-bound Notch are called the extracellular domain (ECD) and the transmembrane fragment comprising of a transmembrane domain and an intracellular domain.

From N-terminus, the ECD consists of 29-36 epidermal growth factor (EGF)-like domains. EGF repeat 12 has been reported to be the main binding domain involved in receptor-ligand interactions. Following the EGF-like repeating domains is the negative regulatory region (NRR), which contains three cysteine-rich Lin12/Notch repeats (LNR) and the HD domain which connects the transmembrane domain and ECD polypeptides to form the Notch heterodimer (NPL 1). The NRR is crucial in mediating auto-inhibition of Notch receptor and prevent activation in the absence of the correct signal (NPL 2).

Notch ligands of the Delta/Serrate/Lag-2 (DSL) family in humans and mice are categorized into 2 classes, depending on whether they are a homolog of the Drosophila Notch ligand Delta or Serrate. The Delta-like ligands (DLL) include DLL1, DLL3, DLL4, and the Serrate homologs include Jagged1 (also called Jag1) and Jagged2 (also called Jag2). Despite functional differences among the four Notch receptors, interaction with either DLL or Jagged ligands leads to the activation of the same canonical signalling pathway (NPL 3).

### Physiological Function of Notch Signalling Especially Role in Stem Cell Signalling and Tissue Regeneration

Notch signalling pathway is recognized as one of the few signalling pathways that are repeatedly used in multiple developmental processes in embryonic and adult tissues. During development, Notch signalling is involved in the stringent control of the balance between the self-renewal and differentiation of various tissue stem cells (SCs) and are responsible for maintaining tissue homeostasis and regeneration of damaged tissues (NPL 3). Context specificity of Notch activation dictates the specific process or functional events that occur (e.g. differentiation, proliferation or apoptosis) and when such events occur (i.e. developmental stages) (NPL 4). This context-dependent Notch activity can therefore drive numerous aspects of the development of multicellular eukaryotic organisms, and has recently been linked to stem cell fate and maintenance in embryonic and adult tissues, including satellite cell, neuronal stem cell, intestinal stem cell and hematopoietic stem cell.

### Limitation of Current Therapeutics

Components of Notch signalling are attractive therapeutic targets as dysregulation of Notch signalling have been implicated in a plethora of developmental disorder and malignancies. However to date, there are several small molecules which demonstrated selectively inhibition of Notch signalling (NPL 5 and 6). gamma-secretase inhibitors (GSIs) which have been used widely to block proteolytic activation of Notch, but they are also known to be non-specific in their action as they also block the processing of more than 90 other substrates, including amyloid precursor protein (APP), E-cadherin and ErbB4 (NPL 7 to 9). In addition, however, such compounds inhibit the proteolysis of multiple transmembrane proteins, including all four Notch receptor (NPL 10) and cause significant toxicities when administered chronically, most notably severe secretory diarrhea that results from colonic goblet cell metaplasia (NPL 11). This gastrointestinal toxicity is believed to be due to the inhibition of NOTCH1 and/or NOTCH2 (NPL 12) which promote the differentiation of progenitor cells in the colonic crypts into absorptive enterocytes.

Apart from small molecules, antagonistic antibodies that are selective to specific Notch receptors have been reported by several groups (NPL 13 to 17). However, most of these Notch antagonistic antibodies are focused on targeting oncology indications and their clinical applications are limited by similar considerations faced by the small molecules inhibitors.

### CITATION LIST

### Non Patent Literature

[NPL 1] Proc Natl Acad Sci USA. 1998 Jul. 7; 95(14):8108
[NPL 2] Nature Structural & Molecular Biology, 14, 295-300(2007)
[NPL 3] Development 2013 140: 689-704
[NPL 4] Cell Death & Disease 8, e2595 (2017)
[NPL 5] Sci Rep. 2019; 9: 10811.
[NPL 6] Methods Mol Biol. 2014; 1187:311-22.
[NPL 7] Nature. 1999 Apr. 8; 398(6727):518-22.
[NPL 8] J Alzheimers Dis. 2011; 25(1):3-28.
[NPL 9] Breast Cancer (Dove Med Press). 2012 Jun. 21; 4:83-90.
[NPL 10] Nature Reviews Molecular Cell Biology, 5, 499-504(2004)
[NPL 11] The Journal of Biological Chemistry, 279, 12876-12882.
[NPL 12] Nature volume 435, pages 964-968(2005)
[NPL 13] Methods. 2012 September; 58(1): 69-78.
[NPL 14] Methods Mol Biol. 2014; 1187:335-42.
[NPL 15] The Journal of Biological Chemistry, 283, 8046-8054.
[NPL 16] Clin Cancer Res; 21(9) May 1, 2015
[NPL 17] Plos One, February 2010, Volume 5, Issue 2, e9094

### SUMMARY OF INVENTION

### Technical Problem

The present inventors have thought that it is challenging to limit the Notch modulating effect to the pathological site while sparing the normal tissue of side effects. As such, improving the site specificity of Notch modulators is one of the potential strategy to circumvent side effects from systemic administration of Notch modulators. The present invention has been made on the basis of such an idea. An objective of the present disclosure is to provide multispecific antigen-binding molecules that enable Notch signaling pathway activation in a cell of interest in a cell anchorage dependent manner, methods for producing the multispecific antigen-binding molecules, and pharmaceutical compositions comprising such a multispecific antigen-binding molecule as an active ingredient for activating Notch signaling pathway in a cell of interest.

There is a previous review that described the core components involved in Notch signalling, and ligand/receptor interaction and triggering of proteolytic activation (Cell. 2009 Apr. 17; 137(2):216-33) According to this report, the authors' theory is as follows: activation of Notch signalling involves: (1) cell-to-cell contacts that allow interactions between Notch receptor and its ligands (Delta like ligands and Jagged ligands); (2) Notch receptor undergoes conformational changes upon ligand engagement resulting in a mechanical force that exposes a protective domain within Notch receptor known as the negative regulatory region (NRR). This will lead to the subsequent proteolytic cleavage of the S2 site and the remaining domains are recognized and cleaved by the constitutively active gamma-secretase at S3 site to release the Notch intracellular domain (NICD); and (3) Nuclear translocation of NICD from membrane leads to its binding to a conserved transcription factor, CSL; CBF1/RBPJ to upregulate Notch target genes.

### Solution to Problem

The inventors found that a multispecific antigen-binding molecule that comprises a first antigen-binding moiety which specifically binds to a Notch receptor on a first target cell, and a second antigen-binding moiety which specifically binds to an anchor antigen on a second target cell, can cause Notch signaling activation in the first target cell when (or only when) the multispecific antigen-binding molecule is binding to the anchor antigen on the second target cell (i.e., anchorage-dependent signaling activation). Tissue or site specificity of Notch signaling pathway activation is conferred by site-specific binding domain's selective binding to a unique anchor antigen whose expression is specific, exclusive or limited to the tissue or cell population of interest. The concept of site specific Notch “trans-activation” can be achieved by adopting such multispecific antibody formats.

More specifically, the present invention provides the following:
[1] A multispecific antigen-binding molecule comprising:
   (i) a first antigen-binding moiety which specifically binds to a Notch receptor on a first target cell, and
   (ii) a second antigen-binding moiety which specifically binds to an anchor antigen on a second target cell,
   wherein the first target cell and the second target cell are different cells, and
   wherein the multispecific antigen-binding molecule activates the Notch signaling pathway in the first target cell when the multispecific antigen-binding molecule is binding to the anchor antigen on the second target cell.
[2] The multispecific antigen-binding molecule of [1], wherein the first target cell is a tissue stem cell, activated CD4 T-lymphocyte, cell secreting pro-fibrotic factors or pro-tumorigenic cell in tumor microenvironment.
[3] The multispecific antigen-binding molecule of [2], wherein the tissue stem cell is a satellite cell, Adult intestinal stem cell or crypt base columnar (CBC) cell.
[4] The multispecific antigen-binding molecule of any one of [1] to [3], wherein the first-binding moiety comprises a Notch-binding domain of a Notch receptor ligand.
[5] The multispecific antigen-binding molecule of [4], wherein the Notch receptor ligand is a ligand to a Notch1, Notch2, Notch3, or Notch4 receptor.
[6] The multispecific antigen-binding molecule of [4] or [5], wherein the Notch receptor ligand is a Delta protein or Jagged protein.
[7] The multispecific antigen-binding molecule of [6], wherein the Delta protein is a Delta Like Ligand 1 (DLL1), DLL3, or DLL4.
[8] The multispecific antigen-binding molecule of [6], wherein the Jagged protein is a Jagged 1 or Jagged 2.
[9] The multispecific antigen-binding molecule of any one of [1] to [3], wherein the first antigen-binding moiety comprises a Fab, scFv, VHH, VL, VH, or single domain antibody that specifically binds to the Notch receptor.
[10] The multispecific antigen-binding molecule of any one of [1] to [9], wherein the second target cell is selected from the group consisting of a muscle cell which is not a satellite cell, activated fibroblast, immune cell that expresses FcgRIIB, GPC3 expressing cancer cell, and cell in the intestinal crypts.
[11] The multispecific antigen-binding molecule of [10], wherein the immune cell that expresses FcgRIIB is selected from the group consisting of a circulating B lymphocyte, monocyte, neutrophil, lymphoid-dendritic cell and myeloid-dendritic cell.
[12] The multispecific antigen-binding molecule of [10], wherein the anchor antigen on the second target cell is selected from the group consisting of Calcium Voltage-Gated Channel Subunit Alpha1 S (CACNA1S), Fibroblast activation protein (FAP), Glypican-3 (GPC3) and Fc gamma RIIB (CD32B).
[13] The multispecific antigen-binding molecule of any one of [1] to [12], wherein the second antigen-binding moiety comprises a Fab, scFv, VHH, VL, VH, single domain antibody, ligand, or engineered Fc region that specifically binds to the anchor antigen.
[14] The multispecific antigen-binding molecule of any one of [1] to [13], wherein the multispecific antigen-binding molecule further comprises an Fc region.
[15] The multispecific antigen-binding molecule of [14], wherein the Fc region is an engineered Fc region which exhibits reduced binding affinity to human Fc gamma receptor, as compared to a native human IgG1 Fc domain.
[16] The multispecific antigen-binding molecule of any one of [13] to [15], wherein the second antigen-binding moiety comprises an engineered Fc region which specifically binds to FcgRIIB.
[17] The multispecific antigen-binding molecule of [16], wherein the multispecific antigen-binding molecule further comprises one more of the first antigen-binding moiety.
[18] The multispecific antigen-binding molecule of [16], wherein the multispecific antigen-binding molecule further comprises a third antigen-binding moiety which specifically binds to an anchor antigen on a third target cell.
[19] The multispecific antigen-binding molecule of [18], wherein the second target cell and the third target cell are different cells or the same cells.
[20] A pharmaceutical composition comprising the multispecific antigen-binding molecule of any one of [1] to [19], and a pharmaceutically acceptable carrier.
[21] A method for activating Notch signaling pathway in a first target cell, comprising contacting the first target cell with an effective amount of the multispecific antigen-binding molecule of any one of [1] to [19], or the pharmaceutical composition of [20].
[22] The method of [21], wherein the first target cell is in a mammalian subject in vivo.
[23] The method of [22], wherein the subject is a human.
[24] An isolated nucleic acid encoding the multispecific antigen-binding molecule of any one of [1] to [19].
[25] A vector comprising the nucleic acid of [24].
[26] A host cell comprising the nucleic acid of [24] or the vector of [25].
[27] A method of producing the multispecific antigen-binding molecule any one of [1] to [19], comprising culturing the host cell of [26].

### BRIEF DESCRIPTION OF DRAWINGS

FIG. 1 Illustration showing the concept of site specific Notch activation with a site specific binding domain and a polypeptide capable of binding to Notch receptor leading to its activation. (A) Bi-specific antibody with one Fab arm capable of binding specifically to an anchor antigen to induce anchorage dependent trans-activation of Notch receptors by the Notch agonist domain. (B) Multi-specific antibody with an engineered Fc capable of binding to an anchor antigen and with two Notch agonist domains. (C) Multi-specific antibody with an engineered Fc capable of binding to an anchor antigen and with an engineered Fc capable of binding to one anchor antigen (1), one Notch agonist domain and an additional binding domain to a second anchor antigen (2) for additional specificity.

FIG. 2 Illustration depicting the concept of Notch agonist antibody with Fc gamma RIIB-selective binding technology(The technology selectively increases binding affinity of the Fc region to inhibitory Fc gamma RIIb over activating Fc gamma receptors including Fc gamma RIIa, Fc gamma RI, and Fc gamma RIIIa by introducing mutations in the Fc region.).

FIG. 3 Preparation of multi-specific Notch agonistic antibody. Illustration of molecule formats and naming rule. (A) anti-AA//Jag-Fc, consists of an Fc, devoid of Fc gamma R binding (“Fc gamma R silenced”), a human Jag1 extra cellular domain (ECD), and a Fab, binding to a target antigen such as GPC3. Heterodimerization and correct assembly are achieved by knob into hole (kih) mutation in the Fc (B) Jag1//Jag1-Fc, consists of an Fc, devoid of Fc gamma R binding, and two human Jag1 extra cellular domain (ECD).

FIG. 4 Anchorage dependent Notch activation induced Notch agonist antibodies. (A) RBP-Jk reporter assay shows the effect of anchorage dependent Notch activation in C212 cells stably expressing the luciferase reporter gene (C2C12-Notch reporter cells) induced by Notch agonist antibodies. Human IgG1 antibody was included as an isotype control. The Notch agonist antibodies (10 microgram (mcg)/mL) were either directly immobilized to culture plate via adsorption, immobilized by anti-human IgG kappa light chain (anti-IgG kappa-LC) antibody (10 mcg/mL) adsorbed on culture plate before seeding C212-Notch reporter cells, or add together with C2C12-Notch reporter cells (no immobilization). Data are represented as luciferase fold stimulation normalized to isotype control antibody treatment. (B) Human IgG1 antibody was included as an isotype control. The Notch agonist antibodies (10 mcg/mL) were either directly immobilized to culture plate via adsorption, immobilized by anti-human IgG Fc specific antibody (10 mcg/mL) that were first adsorbed on culture plate or added with or without anti-anti-human IgG Fc antibody directly to culture medium without immobilization.

FIG. 5 Notch activation is dependent on the availability and level of anchor antigen. (A) SK-HEP1 cells stably overexpressing GPC3 (SK-PCa 60) was co-cultured at indicated cell density with C2C12-Notch reporter cells, treated with either isotype control antibody or anti-GPC3//Jag1-Fc antibody (10 mcg/mL) for 24 hours before performing dual luciferase assay. Data are represented as luciferase fold stimulation normalized to isotype control treatment. (B) SK-HEP1 cells stably overexpressing GPC3 at various levels (High GPC3: SK-PCa 60, Mid GPC3: SK-PCA31 and Low SK-PCA 13) were co-cultured with C2C12-Notch reporter cells, treated with either isotype control antibody or anti-GPC3//Jag1-Fc antibody (10 mcg/mL) for 24 hours before performing dual luciferase assay. Data are represented as luciferase fold stimulation normalized to isotype control treatment.

FIG. 6 qPCR analysis shows the relative expression of Notch target genes, (A) HEY1 and (B) NRARP. Either isotype control antibody or anti-GPC3//Jag1-Fc antibody (0 or 25 mcg/mL) was adsorbed to culture plate overnight before the addition of parental C2C12 cells. The C2C12 cells were further treated with either DMSO or gamma-secretase inhibitor, DAPT (10 micromolar) for 24 hours before cells were harvested for qPCR analysis.

FIG. 7 Anchorage dependent Notch activation induced by bi-specific Notch agonist antibody against site specific anchor antigens. (A) Notch reporter cells were co-cultured with NIH3T3-FAP overexpressing cells and treated with either anti-KLH control antibody or anti-FAP//Jag1 bi-specific antibody (10 mcg/mL) for 24 hours before performing luciferase assay. (B) FACS analysis showing the ability of anti-FAP//Jag1-Fc to bind to surface FAP overexpressed on NIH3T3 cells. (C) Notch reporter cells were co-cultured with MDCK-Fc gamma RIIB overexpressing cells. Jag1//Jag1-Fc* consists of an engineered Fc which preferentially binds to Fc gamma RIIB as anchor antigen.

FIG. 8 Anchorage dependent Notch activation induced by bi-specific Notch agonist antibody with extracellular domain of other Notch ligands (i.e. Jagged2, DLL1, DLL3, DLL4) instead of Jagged1. (A) Bi-specific antibodies (10 mcg/mL) consisting of an Notch agonist arm (i.e. Notch ligand as indicated) and an anti-anchor antigen arm (i.e. anti-KLH or anti-GPC3) were either immobilized by anti-human Fc specific antibody coated on plate or added directly to Notch reporter cells in culture medium (Non-immobilized condition) before performing luciferase assay. Data are represented as fold change of relative luciferase unit (RLU) after normalizing to anti-KLH control antibody treatment. (B) SK-HEP1 cells stably overexpressing GPC3 (SK-PCA 60 and SK-PCA 31) was co-cultured with C2C12-Notch reporter cells, treated with bi-specific antibody (10 mcg/mL) consisting of either an anti-KLH antibody or anti-GPC3 antibody for the anti-anchor antigen binding arm, and the extracellular domain of human Notch ligands as indicated for 24 hours before performing luciferase assay. Data are represented as fold change of relative luciferase unit (RLU) after normalizing to anti-KLH control antibody treatment. (C) Quantification of cell surface GPC3 expressed on GPC3 over-expressing cells (SK-PCA31 and SK-PCA60) using Quantum™ Simply Cellular (registered trademark) (QSC) microspheres kit from Bangs Laboratories. Data was presented as the number of surface GPC3 expressed per cell.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R.I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R.I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

The definitions and detailed description below are provided to facilitate understanding of the present disclosure illustrated herein.

### Definitions

### Amino Acids

Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Alteration of Amino Acids

For amino acid alteration (also described as “amino acid substitution” or “amino acid mutation” within this description) in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations: amino acid(s) at (a) position 33, (b) position 55, (c) position 96, (d) positions 33 and 55, (e) positions 33 and 96, (f) positions 55 and 96, and (g) positions 33, 55, and 96.

Furthermore, herein, as an expression showing alteration of amino acids, an expression that shows before and after a number indicating a specific position, one-letter or three-letter codes for amino acids before and after alteration, respectively, may be used appropriately. For example, the alteration N100bL or Asn100bLeu used when substituting an amino acid contained in an antibody variable region indicates substitution of Asn at position 100b (according to Kabat numbering) with Leu. That is, the number shows the amino acid position according to Kabat numbering, the one-letter or three-letter amino-acid code written before the number shows the amino acid before substitution, and the one-letter or three-letter amino-acid code written after the number shows the amino acid after substitution. Similarly, the alteration P238D or Pro238Asp used when substituting an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number shows the amino acid before substitution, and the one-letter or three-letter amino-acid code written after the number shows the amino acid after substitution.

### Polypeptides

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

### Percent (%) Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Recombinant Methods and Compositions

Antibodies and antigen-binding molecules may be produced using recombinant methods and compositions, e.g., as described in U.S. Pat. No. 4,816,567. In one embodiment, isolated nucleic acid encoding an antibody as described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making the multispecific antigen-binding molecule of the present disclosure is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody described herein, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

Recombinant production of an antigen-binding molecule described herein could be done with methods similar to those described above, by using a host cell comprises (e.g., has been transformed with) one or plural vectors comprising nucleic acid that encodes an amino acid sequence comprising the whole antigen-binding molecule or part of the antigen-binding molecule.

### Antigen-Binding Molecules and Multispecific Antigen-Binding Molecules

The term “antigen-binding molecule”, as used herein, refers to any molecule that comprises an antigen-binding site or any molecule that has binding activity to an antigen, and may further refer to molecules such as a peptide or protein having a length of about five amino acids or more. The peptide and protein are not limited to those derived from a living organism, and for example, they may be a polypeptide produced from an artificially designed sequence. They may also be any naturally-occurring polypeptide, synthetic polypeptide, recombinant polypeptide, and such. Scaffold molecules comprising a known stable conformational structure such as alpha/beta barrel as scaffold, and in which part of the molecule is made into an antigen-binding site, is also one embodiment of the antigen binding molecule described herein.

“Multispecific antigen-binding molecules” refers to antigen-binding molecules that bind specifically to more than one antigen. The term “bispecific” means that the antigen binding molecule is able to specifically bind to at least two distinct antigenic determinants. The term “trispecific” means that the antigen binding molecule is able to specifically bind to at least three distinct antigenic determinants.

In certain embodiments, the multispecific antigen-binding molecule of the present application is a bispecific antigen-binding molecule, i.e. specifically binding to a Notch receptor on a first target cell, and specifically binding to an anchor antigen on a second target cell.

In certain embodiments, the first target cell expressing a Notch receptor and the second target cell expressing an anchor antigen are different cells.

In certain embodiments, the multispecific antigen-binding molecule of the present application is a trispecific antigen-binding molecule, i.e. specifically binding to a Notch receptor on a first target cell, specifically binding to an anchor antigen on a second target cell and specifically binding to an anchor antigen on a third target cell.

In certain embodiments, the first target cell expressing a Notch receptor and the second target cell expressing an anchor antigen are different cells, and the second target cell expressing an anchor antigen and the third target cell expressing an anchor antigen are different cells or the same cells. In certain embodiments, the first target cell expressing a Notch receptor and the third target cell expressing an anchor antigen are different cells.

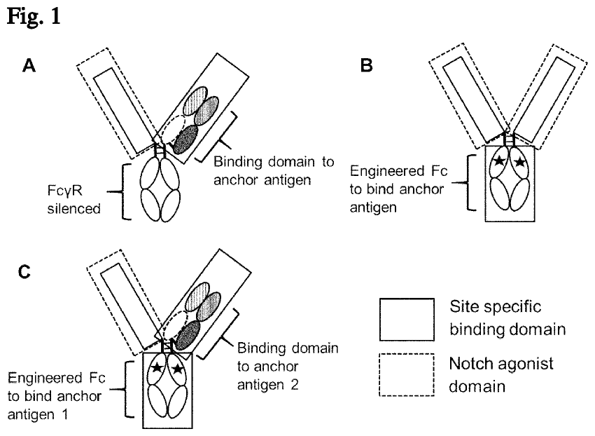

The components of the multispecific antigen-binding molecules of the present disclosure can be fused to each other in a variety of configurations. Exemplary configurations are depicted in FIG. 1.

In some aspects, a multispecific antigen-binding molecule of the present invention comprises at least one first antigen-binding moiety (e.g., Notch agonist domain), e.g., one or two first antigen-binding moieties. In some aspects, a multispecific antigen-binding molecule of the present invention comprises at least one second antigen-binding moiety (e.g., site-specific binding domain), e.g., one or two second antigen-binding moieties.

In one aspect, the present disclosure provides a multispecific antigen-binding molecule comprising:
(i) a first antigen-binding moiety which specifically binds to a Notch receptor on a first target cell, and
(ii) a second antigen-binding moiety which specifically binds to an anchor antigen on a second target cell,
   wherein the first target cell and the second target cell are different cells, and the multispecific antigen-binding molecule trans-activates the Notch signaling pathway in the first target cell. In one aspect, the feature, “the multispecific antigen-binding molecule trans-activates the Notch signaling pathway in the first target cell” is alternatively referred to as follows: “the multispecific antigen-binding molecule activates the Notch signaling pathway in the first target cell when (or only when) the multispecific antigen-binding molecule is binding to the anchor antigen on the second target cell. Preferably, the anchor antigen on the second target cell is not expressed (or not significantly/substantially/specifically expressed) in the first target cell.

In one aspect, the present disclosure provides a multispecific antigen-binding molecule comprising:
(i) a first antigen-binding moiety which specifically binds to a Notch receptor on a first target cell, and
(ii) a second antigen-binding moiety which specifically binds to an anchor antigen on a second target cell,
   wherein the first target cell and the second target cell are different cells.

In certain aspect, the present disclosure provides a multispecific antigen-binding molecule further comprises an Fc region.

In certain embodiment, the Fc region can be an Fc region which exhibits reduced binding affinity to human Fc gamma receptor, as compared to a native human IgG1 Fc domain. In certain embodiment, the Fc region can be an engineered Fc region which exhibits an ability to bind to an Fc gamma receptor that is reduced compared to the ability of the Fc region of a wild-type IgG antibody of the same isotype as the multispecific antigen-binding molecule to bind to the Fc gamma receptor.

In one aspect, the present disclosure provides a multispecific antigen-binding molecule comprising:
(i) a first antigen-binding moiety which specifically binds to a Notch receptor on a first target cell, and
(ii) a second antigen-binding moiety which specifically binds to an anchor antigen on a second target cell,
   wherein the first target cell and the second target cell are different cells, and the multispecific antigen-binding molecule trans-activates the Notch signaling pathway in the first target cell.

In one aspect, the feature, “the multispecific antigen-binding molecule trans-activates the Notch signaling pathway in the first target cell” is alternatively referred to as follows: “the multispecific antigen-binding molecule activates the Notch signaling pathway in the first target cell when (or only when) the multispecific antigen-binding molecule is binding to the anchor antigen on the second target cell. Preferably, the anchor antigen on the second target cell is not expressed (or not significantly/substantially/specifically expressed) in the first target cell.

In one embodiment, the second antigen-binding moiety comprises an engineered Fc region which specifically binds to an anchor antigen on a second target cell. In one embodiment, the second antigen-binding moiety comprises an engineered Fc region which specifically binds to FcgRIIB as an anchor antigen. In certain embodiment, the multispecific antigen-binding molecule further comprises one more of the first antigen-binding moiety.

In one aspect, the present disclosure provides a multispecific antigen-binding molecule comprising:
(i) a first antigen-binding moiety which specifically binds to a Notch receptor on a first target cell, and
(ii) a second antigen-binding moiety which specifically binds to an anchor antigen on a second target cell,
   wherein the first target cell and the second target cell are different cells, and the multispecific antigen-binding molecule trans-activates the Notch signaling pathway in the first target cell. In one aspect, the feature, “the multispecific antigen-binding molecule trans-activates the Notch signaling pathway in the first target cell” is alternatively referred to as follows: “the multispecific antigen-binding molecule activates the Notch signaling pathway in the first target cell when (or only when) the multispecific antigen-binding molecule is binding to the anchor antigen on the second target cell. Preferably, the anchor antigen on the second target cell is not expressed (or not significantly/substantially/specifically expressed) in the first target cell.

In one embodiment, the second antigen-binding moiety comprises an engineered Fc region which specifically binds to an anchor antigen on a second target cell. In one embodiment, the second antigen-binding moiety comprises an engineered Fc region which specifically binds to FcgRIIB as an anchor antigen. In one embodiment, the multispecific antigen-binding molecule further comprises a third antigen-binding moiety which specifically binds to an anchor antigen on a third target cell.

In certain embodiment, the second target cell and the third target cell can be different cells or the same cells. In certain embodiments, the first target cell expressing a Notch receptor and the third target cell expressing an anchor antigen are different cells.

According to any of the above embodiments, components of the multispecific antigen-binding molecules (e.g. antigen binding moiety, Fc region (“Fc domain”)) may be fused directly or through various linkers, particularly peptide linkers comprising one or more amino acids, typically about 2-20 amino acids, that are described herein or are known in the art. Suitable, non-immunogenic peptide linkers include, for example, (G4S)n, (SG4)n, (G4S)n or G4(SG4)n peptide linkers, wherein n is generally a number between 1 and 10, typically between 2 and 4.

In one aspect, the present disclosure provides a multispecific antigen-binding molecule comprising:
(i) a first antigen-binding moiety that specifically binds to Notch recpetor; and
(ii) a second antigen-binding moiety that specifically binds to an anchor antigen;
wherein the first antigen-binding moiety and the second antigen-binding moiety comprises antibody variable regions each, wherein the first antibody variable region of the first antigen-binding moiety is fused with a first heavy chain constant region, the second antibody variable region of the first antigen-binding moiety is fused with a first light chain constant region, the third antibody variable region of the second antigen-binding moiety is fused with a second heavy chain constant region, the fourth antibody variable region of the second antigen-binding moiety is fused with a second light chain constant region.

### Pyroglutamylation

It is known that when an antibody is expressed in cells, the antibody is modified after translation. Examples of the posttranslational modification include cleavage of lysine at the C terminal of the heavy chain by a carboxypeptidase; modification of glutamine or glutamic acid at the N terminal of the heavy chain and the light chain to pyroglutamic acid by pyroglutamylation; glycosylation; oxidation; deamidation; and glycation, and it is known that such posttranslational modifications occur in various antibodies (Journal of Pharmaceutical Sciences, 2008, Vol. 97, p. 2426-2447).

The multispecific antigen-binding molecules of the present disclosure also includes a multispecific antibody which has undergone posttranslational modification. Examples of the multispecific antigen-binding molecules thereof of the present disclosure, which undergoes posttranslational modification, include multispecific antibodies which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

Antigen Binding Moiety which Specifically Binds to Notch Receptor

As used herein, the term “antigen binding moiety” refers to a polypeptide molecule that specifically binds to an antigen. In one embodiment, an antigen binding moiety is able to direct the entity to which it is attached to a target site, for example to a specific type of cell expressing a Notch receptor.

In another embodiment an antigen binding moiety which specifically binds to Notch receptor is able to activate signaling through Notch receptor, for example a Notch signaling pathway in an anchor antigen dependent manner. Antigen binding moieties may include antibodies, fragments thereof, ligands as further defined herein.

In certain embodiments, antigen binding moieties may include an antigen binding domain or an antibody variable region of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: alpha, delta, epsilon, gamma, or mu. Useful light chain constant regions include any of the two isotypes: kappa and lambda.

In one aspect, the present disclosure provides a multispecific antigen-binding molecule comprising:
(i) a first antigen-binding moiety which specifically binds to a Notch receptor on a first target cell, and
(ii) a second antigen-binding moiety which specifically binds to an anchor antigen on a second target cell,
wherein the first target cell and the second target cell are different cells, and the multispecific antigen-binding molecule trans-activates the Notch signaling pathway in the first target cell. In one aspect, the feature, “the multispecific antigen-binding molecule trans-activates the Notch signaling pathway in the first target cell” is alternatively referred to as follows: “the multispecific antigen-binding molecule activates the Notch signaling pathway in the first target cell when (or only when) the multispecific antigen-binding molecule is binding to the anchor antigen on the second target cell. Preferably, the anchor antigen on the second target cell is not expressed (or not significantly/substantially/specifically expressed) in the first target cell.

As used herein, the terms “first”, “second”, and “third” with respect to antigen binding moieties etc., are used for convenience of distinguishing when there is more than one of each type of moiety and such. Use of these terms is not intended to confer a specific order or orientation of the multispecific antigen-binding molecule unless explicitly so stated.

In one aspect, the first antigen-binding moiety which specifically binds to a Notch receptor of the present disclosure includes any polypeptide which can bind and activate Notch signaling in a first target cell in an anchorage dependent manner (i.e. concurrent binding of site specific binding domain to its anchor antigen).

In certain embodiments, the Notch receptor antigen-binding moiety (“first antigen-binding moiety”) is generally a Fab molecule, particularly a conventional Fab molecule. In certain embodiments, the Notch receptor antigen-binding moiety (“first antigen-binding moiety”) is “single chain Fv (scFv)”, “single chain antibody”, “Fv”, “single chain Fv 2 (scFv2)”, “Fab”, “F(ab′)2”, VHH, VL, VH, single domain antibody, or any antibody fragment.

In certain embodiments, the Notch receptor antigen-binding moiety (“first antigen-binding moiety”) comprises a Notch-binding domain of a Notch receptor ligand. In certain embodiments, the Notch receptor ligand is a ligand to a Notch1, Notch2, Notch3, or Notch4 receptor. Herein below, Genbank or RefSeq registration numbers are shown within parentheses. In certain embodiments, RefSeq registration numbers of the human Notch receptors are as follows: Notch1 (NP_060087.3 or P46531), Notch2 (NP_077719.2 (isoform 1) or NP_001186930.1 (isoform 2)), Notch3 (NP_000426.2), or Notch4 (NP_004548.3 or Q99466). In certain embodiments, the Notch receptor ligand is a Delta protein or Jagged protein. In certain embodiment, an extra cellular domain (ECD) of any of those ligands disclosed herein can be used as a Notch-binding domain. In certain embodiments, the Delta protein is a Delta Like Ligand 1 (DLL1) (GenBank Accession Nos. ABC26875 or NP005609; RefSeq NP_005609.3), DLL3 (GenBank Accession Nos./RefSeq NP_982353.1 or NP_058637.1), or DLL4 (GenBank Accession Nos. NP_982353.1; RefSeq NP_061947.1), homologs, or functional (Notch binding) variants, fragments, or derivatives thereof. In certain embodiments, the Delta protein is Delta Like Ligand 1 (DLL1) or DLL4. In certain embodiments, the Jagged protein is a Jagged 1 (GenBank Accession No. AAC51731; RefSeq NP_000205.1) or Jagged 2 (GenBank Accession No. AAD15562; RefSeq NP_002217.3 (isoform A) or NP_660142.1 (isoform B)), homologs, or functional (Notch binding) variants, fragments, or derivatives thereof. In certain embodiment, a human Jagged 1 ECD shown as a partial sequence of SEQ ID NO: 3 or 4 can be used as a Notch-binding domain.

In certain embodiments, the Notch receptor antigen-binding moiety (“first antigen-binding moiety”) specifically binds to the whole or a portion of a partial peptide of the Notch receptor. In a particular embodiment, the Notch receptor is human Notch receptor or cynomolgus Notch receptor or mouse Notch receptor, most particularly human Notch receptor. In a particular embodiment, the Notch receptor antigen-binding moiety (“first antigen-binding moiety”) is cross-reactive for (i.e. specifically binds to) human and cynomolgus Notch receptors.

The multispecific antigen-binding molecules of the present disclosure also include a multispecific antibody which has undergone posttranslational modification. Examples of the multispecific antigen-binding molecules thereof of the present disclosure, which undergoes posttranslational modification, include multispecific antigen-binding molecules which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

Antigen-Binding Moiety which Specifically Binds to an Anchor Antigen

In one aspect, the multispecific antigen-binding molecule described herein comprises at least one antigen-binding moiety capable of binding to an anchor antigen (also referred to herein as an “anchor antigen-binding moiety” or “second antigen-binding moiety”). In certain embodiments, the multispecific antigen-binding molecule comprises one antigen-binding moiety capable of binding to Calcium Voltage-Gated Channel Subunit Alpha1 S (CACNA1S), Fibroblast activation protein (FAP), Glypican-3 (GPC3) or Fc gamma RIIB (CD32B).

In one aspect, the second antigen-binding moiety which specifically binds to an anchor antigen of the present disclosure includes any polypeptide which can bind the anchor antigen as long as the multispecific antigen-binding molecule of the present disclosure can trans-activate the Notch signaling pathway in the first target cell.

In certain embodiments, the anchor antigen-binding moiety (“second antigen-binding moiety”) is generally a Fab molecule, particularly a conventional Fab molecule. In certain embodiments, the anchor antigen-binding moiety (“second antigen-binding moiety”) is a domain comprising antibody light-chain and heavy-chain variable regions (VL and VH). In certain embodiments, the anchor antigen-binding moiety (“second antigen-binding moiety”) is “single chain Fv (scFv)”, “single chain antibody”, “Fv”, “single chain Fv 2 (scFv2)”, “Fab”, “F(ab′)2”, VHH, VL, VH, single domain antibody or any antibody fragment.

In certain embodiments, the anchor antigen-binding moiety (“second antigen-binding moiety”) specifically binds to the whole or a portion of a partial peptide of the anchor antigen. In a particular embodiment, the anchor antigen is a human anchor antigen or cynomolgus anchor antigen or mouse anchor antigen, most particularly a human anchor antigen. In a particular embodiment, the anchor antigen-binding moiety (“second antigen-binding moiety”) is cross-reactive for (i.e. specifically binds to) human and cynomolgus monkey anchor antigens.

In one aspect, the present disclosure provides a multispecific antigen-binding molecule comprising:
(i) a first antigen-binding moiety which specifically binds to a Notch receptor on a first target cell, and
(ii) a second antigen-binding moiety which specifically binds to an anchor antigen on a second target cell,
wherein the first target cell and the second target cell are different cells, and the multispecific antigen-binding molecule trans-activates the Notch signaling pathway in the first target cell. In one aspect, the feature, “the multispecific antigen-binding molecule trans-activates the Notch signaling pathway in the first target cell” is alternatively referred to as follows: “the multispecific antigen-binding molecule activates the Notch signaling pathway in the first target cell when (or only when) the multispecific antigen-binding molecule is binding to the anchor antigen on the second target cell. Preferably, the anchor antigen on the second target cell is not expressed (or not significantly/substantially/specifically expressed) in the first target cell.

In specific embodiments, the second antigen-binding moiety of the present disclosure specifically binds to GPC3, and the GPC3 antigen-binding moiety (“second antigen-binding moiety”) comprises the combinations of H-chain CDR 1, CDR 2, and CDR 3 and L-chain CDR 1, CDR 2, and CDR 3 of (b1) below:
(b1) a heavy chain variable region comprising the complementarity determining region (CDR) 1, the CDR 2, and the CDR 3 comprised in SEQ ID NO: 7, and a light chain variable region comprising the CDR 1, the CDR 2, and the CDR 3 comprised in SEQ ID NO: 8.

In specific embodiments, the GPC3 antigen-binding moiety (“second antigen-binding moiety”) comprises the antibody variable regions that comprise human antibody frameworks or humanized antibody frameworks.

In specific embodiments, the GPC3 antigen-binding moiety (“second antigen-binding moiety”) comprises (d1) below:
(d1) a heavy chain variable region comprising the amino acid sequence of SEQ ID NO: 7, and a light chain variable region comprising the amino acid sequence of SEQ ID NO: 8.

In one embodiment, the GPC3 antigen-binding moiety (“second antigen-binding moiety”) comprises a heavy chain variable region sequence that is at least about 95%, 96%, 97%, 98%, 99% or 100% identical to SEQ ID NO: 7 and a light chain variable region sequence that is at least about 95%, 96%, 97%, 98%, 99% or 100% identical to SEQ ID NO: 8.

In specific embodiments, an antigen-binding molecule of the present invention comprises:
the sequence of SEQ ID NO: 5 (“chain 1” which comprises a variable heavy chain domain (VH) and a constant heavy chain domain 1 (CH1) (site-specific binding domain), and an Fc region);
the sequence of SEQ ID NO: 6 (“Chain 2” which comprises a variable light chain domain (VL) (site-specific binding domain) and a constant light chain domain (CL)); and
the sequence of SEQ ID NO: 3 (“Chain 3” which comprises a Jag1 ECD and an Fc region).

In specific embodiments, an antigen-binding molecule of the present invention comprises:
the sequence of SEQ ID NO: 5 (“chain 1” which comprises a variable heavy chain domain (VH) and a constant heavy chain domain 1 (CH1) (site-specific binding domain), and an Fc region);
the sequence of SEQ ID NO: 6 (“Chain 2” which comprises a variable light chain domain (VL) (site-specific binding domain) and a constant light chain domain (CL)); and
the sequence of SEQ ID NO: 25 (“Chain 3” which comprises a Jag2 ECD and an Fc region).

In specific embodiments, an antigen-binding molecule of the present invention comprises:
the sequence of SEQ ID NO: 5 (“chain 1” which comprises a variable heavy chain domain (VH) and a constant heavy chain domain 1 (CH1) (site-specific binding domain), and an Fc region);
the sequence of SEQ ID NO: 6 (“Chain 2” which comprises a variable light chain domain (VL) (site-specific binding domain) and a constant light chain domain (CL)); and
the sequence of SEQ ID NO: 26 (“Chain 3” which comprises a DLL1 ECD and an Fc region).

In specific embodiments, an antigen-binding molecule of the present invention comprises:
the sequence of SEQ ID NO: 5 (“chain 1” which comprises a variable heavy chain domain (VH) and a constant heavy chain domain 1 (CH1) (site-specific binding domain), and an Fc region);
the sequence of SEQ ID NO: 6 (“Chain 2” which comprises a variable light chain domain (VL) (site-specific binding domain) and a constant light chain domain (CL)); and
the sequence of SEQ ID NO: 27 (“Chain 3” which comprises a DLL3 ECD and an Fc region).

In specific embodiments, an antigen-binding molecule of the present invention comprises:
the sequence of SEQ ID NO: 5 (“chain 1” which comprises a variable heavy chain domain (VH) and a constant heavy chain domain 1 (CH1) (site-specific binding domain), and an Fc region);
the sequence of SEQ ID NO: 6 (“Chain 2” which comprises a variable light chain domain (VL) (site-specific binding domain) and a constant light chain domain (CL)); and
the sequence of SEQ ID NO: 28 (“Chain 3” which comprises a DLL4 ECD and an Fc region).

In specific embodiments, the second antigen-binding moiety of the present disclosure specifically binds to FAP, and the FAP antigen-binding moiety (“second antigen-binding moiety”) comprises the combinations of H-chain CDR 1, CDR 2, and CDR 3 and L-chain CDR 1, CDR 2, and CDR 3 of (b1) below:
(b1) a heavy chain variable region comprising the complementarity determining region (CDR) 1, the CDR 2, and the CDR 3 comprised in SEQ ID NO: 31, and a light chain variable region comprising the CDR 1, the CDR 2, and the CDR 3 comprised in SEQ ID NO: 32.

In specific embodiments, the FAP antigen-binding moiety (“second antigen-binding moiety”) comprises the antibody variable regions that comprise human antibody frameworks or humanized antibody frameworks.

In specific embodiments, the FAP antigen-binding moiety (“second antigen-binding moiety”) comprises (d1) below:
(d1) a heavy chain variable region comprising the amino acid sequence of SEQ ID NO: 31, and a light chain variable region comprising the amino acid sequence of SEQ ID NO: 32.

In one embodiment, the FAP antigen-binding moiety (“second antigen-binding moiety”) comprises a heavy chain variable region sequence that is at least about 95%, 96%, 97%, 98%, 99% or 100% identical to SEQ ID NO: 31 and a light chain variable region sequence that is at least about 95%, 96%, 97%, 98%, 99% or 100% identical to SEQ ID NO: 32.

In specific embodiments, an antigen-binding molecule of the present invention comprises:
the sequence of SEQ ID NO: 22 (“chain 1” which comprises a variable heavy chain domain (VH) and a constant heavy chain domain 1 (CH1) (site-specific binding domain), and an Fc region);
the sequence of SEQ ID NO: 23 (“Chain 2” which comprises a variable light chain domain (VL) (site-specific binding domain) and a constant light chain domain (CL)); and
the sequence of SEQ ID NO: 3 (“Chain 3” which comprises a Jag1 ECD and an Fc region).

In specific embodiments, the second antigen-binding moiety of the present disclosure specifically binds to Fc gamma RIIB, and comprises (d1) below:
(d1) an amino acid sequence that is at least about 95%, 96%, 97%, 98%, 99% or 100% identical to SEQ ID NO: 30; or
(d2) an amino acid sequence of SEQ ID NO: 30

The multispecific antigen-binding molecules of the present disclosure also include a multispecific antibody which has undergone posttranslational modification. Examples of the multispecific antigen-binding molecules thereof of the present disclosure, which undergoes posttranslational modification, include multispecific antibodies which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

### Antigen

As used herein, the term “antigen” refers to the whole of or a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g. Notch receptor such as Notch1, Notch 2, Notch3, and Notch4, Calcium Voltage-Gated Channel Subunit Alpha1 S (CACNA1S), Fibroblast activation protein (FAP), Glypican-3 (GPC3) and Fc gamma RIIB (CD32B), etc.) can be any native form the proteins from any vertebrate source, including mammals such as primates (e.g. humans) and rodents (e.g. mice and rats), unless otherwise indicated. In a particular embodiment, the antigen is a human Notch receptor, human CACNA1S human FAP, human GPC3, or human CD32B. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

### Notch Receptor

The term “Notch receptor”, as used herein, refers to any native Notch receptor and homologs of which are known by those skilled in the art from any vertebrate source, including mammals such as primates (e.g. humans) and rodents (e.g., mice and rats), unless otherwise indicated. The amino acid sequence of human Notch receptor1 (also called Notch1) is shown in Genbank Accession No. P46531, the amino acid sequence of human Notch receptor2 (also called Notch2) is shown in Genbank Accession No. AAH71562.2, the amino acid sequence of human Notch receptor3 (also called Notch3) is shown in Genbank Accession No. AAB91371.1, and the amino acid sequence of human Notch receptor4 (also called Notch4) is shown in Genbank Accession No. AAC63097.1.

The term “Notch signaling pathway”, as used herein, refers to the cell-signaling cascade that occurs from the proteolytic cleavage of the expressed mature Notch receptors in a cell membrane due to interaction between Notch proteins and relevant proteins such as Jagged or Delta proteins.

In one embodiment, the anchor antigen on a second target cell can be any relevant antigen as long as the multispecific antigen-binding molecule can trans-activate the Notch signaling pathway in the first target cell.

In one embodiment, the anchor antigen on a second target cell preferably is not expressed on a first target cell. In some embodiments, the anchor antigen on a second target cell preferably is not significantly/substantially/specifically expressed on a first target cell. The phrase “not significantly/substantially/specifically expressed”, as used herein, refers to the expression of a protein such as anchor antigen at a level of expression that includes non-significant, non-substantial, non-specific, or background expression but does not include significant, substantial, or specific expression. Whether or not the expression is significant, substantial, specific, or background can be suitably measured by a skilled person. The level of non-significant, non-substantial, non-specific, or background expression may be zero, or may not be zero but near zero, or may be very low enough to be technically neglected by those skilled in the art. For those skilled in the art, the phrase “not expressed” can have the same meaning as the phrase “not significantly/substantially/specifically expressed”.

In some embodiments, a suitable ratio of the first antigen to the anchor antigen on the first target cell that is capable of exhibiting an agonistic activity on a multispecific antigen-binding molecule of the present disclosure can be determined in view of the disclosure provided herewith as well as knowledge available in the art. Therefore, any ratio between the first antigen to the anchor antigen on the first target cell, while not expressly indicated herewith, should still be considered within the scope of the present disclosure as long as such a ratio is capable of providing improvement (e.g. at least 2.5-, 5-, 10-, 20-, 30-, 40-, 50-, 60-, 70-, 80-, 90-, 100-, 150-, 200-, 250-, 300-, 400-, 500-, 600-, 700-, 800-, 900-, 1,000-, or more folds) in the activity of a multispecific antigen-binding molecule in modulating a target signaling pathway as compared to using an antigen-binding moiety specific to either one of first and anchor antigens.

Some examples of a ratio of the first antigen to the anchor antigen on the first target cell ranges about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 50:1, 100:1, 200:1, 500:1, or 1000:1 or more.

In certain embodiments, the multispecific antigen-binding molecule described herein binds to an epitope of Notch receptor, CACNA1S, FAP, GPC3, or CD32B that is conserved among the Notch receptor, CACNA1S, FAP, GPC3, or CD32B proteins from different species

### CACNA1S (Cav1.1, Calcium Channel, Voltage-Dependent, L Type, Alpha 1S Subunit)

CACNA1S also known as the calcium channel, voltage-dependent, L type, alpha 1S subunit, (Cav1.1), is a protein which in humans is encoded by the CACNA1S gene. It is also known as CACNL1A3 and the dihydropyridine receptor (DHPR, so named due to the blocking action DHP has on it). This gene encodes one of the five subunits of the slowly inactivating L-type voltage-dependent calcium channel in skeletal muscle cells. Mutations in this gene have been associated with hypokalemic periodic paralysis, thyrotoxic periodic paralysis and malignant hyperthermia susceptibility. Cav1.1 is a voltage-dependent calcium channel found in the transverse tubule of muscles. In skeletal muscle it associates with the ryanodine receptor RyR1 of the sarcoplasmic reticulum via a mechanical linkage. It senses the voltage change caused by the end-plate potential from nervous stimulation and propagated by sodium channels as action potentials to the T-tubules.

### FAP (Fibroblast Activation Protein, Alpha)

Fibroblast activation protein alpha (FAP-alpha) also known as prolyl endopeptidase FAP is an enzyme that in humans is encoded by the FAP gene. Prolyl endopeptidase FAP is a 170 kDa membrane-bound gelatinase. FAP is a 760 amino acid long type II transmembrane glycoprotein. It contains a very short cytoplasmic N terminal part (6 amino acids), a transmembrane region (amino acids 7-26), and a large extracellular part with an alpha/beta-hydrolase domain and an eight-bladed beta-propeller domain. A soluble form of FAP, which lacks the intracellular and transmembrane part, is present in blood plasma. FAP is a non-classical serine protease, which belongs to the S9B prolyl oligopeptidase subfamily.

### GPC3

GPC3 gene whose nucleotide sequence is disclosed in RefSeq accession number NM_001164617.1 and GPC3 protein is shown in RefSeq accession number NP_001158089.1. Methods for producing an antibody with desired binding activity are known to those skilled in the art. Below is an example that describes a method for producing an antibody (anti-GPC3 antibody) that binds to Glypican-3 (hereinafter, also referred to as GPC3), which belongs to the GPI-anchored receptor family (Int J Cancer. (2003) 103(4), 455-65). Specifically, monoclonal antibodies are prepared as mentioned below. Anti-GPC3 antibodies can be obtained as polyclonal or monoclonal antibodies using known methods. The anti-GPC3 antibodies preferably produced are monoclonal antibodies derived from mammals. Such mammal-derived monoclonal antibodies include antibodies produced by hybridomas or host cells transformed with an expression vector carrying an antibody gene by genetic engineering techniques.

Monoclonal antibody-producing hybridomas can be produced using known techniques, for example, as described below. Specifically, mammals are immunized by conventional immunization methods using a GPC3 protein as a sensitizing antigen. Resulting immune cells are fused with known parental cells by conventional cell fusion methods. Then, hybridomas producing an anti-GPC3 antibody can be selected by screening for monoclonal antibody-producing cells using conventional screening methods.

### Anchor Antigen on a Third Target Cell

The multispecific antigen-binding molecule of the present invention may further comprises a third antigen-binding moiety binding to an anchor antigen on a third target cell. As mentioned herein, tissue/site specificity of Notch signaling pathway activation is achieved by specific binding domain's selective binding to an anchor antigen with specific, exclusive or limited expression in the tissue or cell population of interest. An objective of the third antigen-binding moiety is to further increase the specificity of Notch signaling pathway activation in an anchorage dependent manner. The third target cell and the anchor antigen on the third target cell may be suitably selected by a skilled person such that the above objective is achieved. In some embodiments, the second target cell and the third target cell are different cells, and the anchor antigen on the second target cell is different from the anchor antigen on the third target cell. In some embodiments, the second target cell and the third target cell are the same cells, and the anchor antigen on the second target cell is different from the anchor antigen on the third target cell. In some embodiments, the first target cell expressing a Notch receptor and the third target cell expressing the anchor antigen are different cells.

### Trans-Activation

The terms such as “trans-activation”, “trans-activate(s)”, and “trans-activating” (and other grammatical variations) refer to the feature of a multispecific antigen-binding molecule of the present invention where the multispecific antigen-binding molecule can lead to Notch signal activation on a first target cell when (or only when) the multispecific antigen-binding molecule is binding to an anchor antigen expressed on a second target cell. The concept is that the Notch signaling is activated when the two types of cells, i.e., the first and second target cells, are spatially “linked” or “connected” (i.e., the wording “trans” implies this spatial “linkage” or “connection”) with the multispecific antigen-binding molecule. Preferably, the anchor antigen expressed on the second target cell is not expressed (or not significantly/substantially/specifically expressed) on the first target cell. In some embodiments, the multispecific antigen-binding molecule trans-activates Notch signaling pathway in the first target cell when (or only when) the multispecific antigen-binding molecule is binding to the anchor antigen on the second target cell, where the anchor antigen is, preferably, not expressed (or not significantly/substantially/specifically expressed) on the first target cell. The above description focuses on Notch signaling activation which depends on the anchor antigen on the second target cell (i.e., “anchorage-dependent” trans-activation of the Notch signaling pathway); however, the same can apply to other anchor antigens on target cells for anchorage, such as an anchor antigen on a third target cell.

In some aspects, the multispecific antigen-binding molecule of the present disclosure functions as an agonist and activate a signaling pathway of interest (or a target signaling pathway). In certain embodiments, the multispecific antigen-binding molecule functioning as an agonist of the target signaling pathway upregulates (e.g., stimulates, enhances, promotes or increases) the activity of the target signaling pathway by at least 2.5-, 5-, 10-, 20-, 30-, 40-, 50-, 60-, 70-, 80-, 90-, 100-, 150-, 200-, 250-, 300-, 400-, 500-, 600-, 700-, 800-, 900-, 1,000-, or more folds, or any values of folds between them as compared to using an antigen-binding moiety specific to either one of first and second antigens. The activity of the target signaling, in some embodiments, can be measured via a reporter assay that is responsive to the activation of the signaling pathway.

In some embodiments, a suitable threshold level of a second antigen (or a third antigen) expression on the surface of a second target cell (or a third target cell) that is capable of exhibiting a agonistic activity on a multispecific antigen-binding molecule activity of the present disclosure can be determined in view of the disclosure provided herewith as well as knowledge available in the art. Therefore, any level of expression of the second antigen (or the third antigen) on the second target cell (or the third target cell), while not expressly indicated herewith, should still be considered within the scope of the present disclosure as long as such a threshold is capable of providing improvement (e.g. at least 2.5-fold or more) in the activity of a multispecific antigen-binding molecule in modulating a target signaling pathway as compared to that of an antigen-binding moiety specific to either one of first and second antigens (or third antigen). Some examples of a threshold expression level of a second antigen (or a third antigen) on the surface of second target cell (or third target cell) ranges about 100, 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 11,000, 12,000, 13,000, 14,000, 15,000, 16,000, 17,000, 18,000, 19,000, 20,000, 25,000, 30,000, 40,000, 50,000, 100,000, 200,000, 500,000, or more copies or any intervening number of copies per cell.

### Target Cells and Anchor Antigens

Tissue or site specificity of Notch signaling pathway activation can be achieved by specific binding of the multispecific antigen-binding molecule to a molecule of interest (e.g., a Notch receptor) on a first target cell (via a first antigen-binding moiety) and an anchor antigen on a second target cell (via a second antigen-binding moiety), and optionally, an anchor antigen on a third target cell (via a third antigen-binding moiety), and so on. Preferably, the expression of the anchor antigen is specific, exclusive or limited to the second (or third) target cell. Tissue/site specific trans-activation of the signaling pathway can be achieved by suitably selecting combinations of target cells and anchor antigens as described herein. In some embodiments, neither of the first target cell or the second target cell is in a tumor microenvironment, i.e., both of the first target cell and the second target cell are in a non-tumor microenvironment. In some embodiments, neither of the first target cell or the second target cell is a tumor cell, i.e., both of the first target cell and the second target cell are non-tumor cells. In some embodiment, neither of the first target cell or the second target cell is a non-tumor cell within the tumor microenvironment, i.e. both of the first target cell and the second target cell are non-tumor cells in a non-tumor environment. In some embodiment, the Notch signaling pathway is not anti-oncogenic, i.e., the Notch signaling pathway is non-anti-oncogenic. In some embodiments, the notch signaling pathway is anti-inflammatory, i.e. the notch signaling pathway is not pro-inflammatory, i.e. the Notch signaling pathway is non-inflammatory. The term “tumor microenvironment” refers to a small environment that includes normal cells, molecules, and vessels surrounding a tumor cell, and can affect the growth, proliferation, and/or migration of the tumor cell. In some embodiments, the anchor antigen bound by the second antigen-binding moiety of the present invention is not a specific antigen for tumor cells such as CD33, CD326, CD133 or mesothelin. In some embodiments, the anchor antigen bound by the second antigen-binding moiety of the present invention is not an extracellular antigen or substrate present in the tumour microenvironment, such as collagen.

In one aspect, the multispecific antigen-binding molecule comprising:
(i) a first antigen-binding moiety which specifically binds to a Notch receptor on a first target cell, and
(ii) a second antigen-binding moiety which specifically binds to an anchor antigen on a second target cell,
wherein the first target cell and the second target cell are different cells, and the multispecific antigen-binding molecule trans-activates the Notch signaling pathway in the first target cell.

In one aspect, the feature, “the multispecific antigen-binding molecule trans-activates the Notch signaling pathway in the first target cell” is alternatively referred to as follows: “the multispecific antigen-binding molecule activates the Notch signaling pathway in the first target cell when (or only when) the multispecific antigen-binding molecule is binding to the anchor antigen on the second target cell. Preferably, the anchor antigen on the second target cell is not expressed (or not significantly/substantially/specifically expressed) in the first target cell.

In one embodiment, the first target cell expressing a Notch receptor can be any relevant cell as long as the multispecific antigen-binding molecule can trans-activate the Notch signaling pathway in the first target cell.

In some embodiments, the first target cell expressing a Notch receptor is a tissue stem cell (also known as ‘tissue-specific stem cell’ or ‘Adult stem cell’), activated CD4 T-lymphocyte, cell secreting pro-fibrotic factors or pro-tumorigenic cell in tumor microenvironment. In one such embodiment, the tissue stem cell is a satellite cell, adult intestinal stem cell or crypt base columnar (CBC) cell.

In one aspect, the second antigen-binding moiety specifically binds to an anchor antigen on a second target cell.

In one embodiment, the second target cell expressing an anchor antigen can be any relevant cell as long as the multispecific antigen-binding molecule can trans-activate the Notch signaling pathway in the first target cell in the anchor antigen dependent manner.

In one embodiment, the second target cell is selected from the group consisting of a muscle cell which is not a satellite cell, activated fibroblast, immune cell that expresses FcgRIIB, GPC3 expressing cancer cell, and cell in the intestinal crypts.

In some embodiments, the immune cell that expresses FcgRIIB is selected from the group consisting of a circulating B lymphocyte, monocyte, neutrophil, lymphoid-dendritic cell and myeloid-dendritic cell.

A person skilled in the art can readily select any appropriate anchor antigen and second target cell for the design and implementation of the second antigen-binding moiety according to common general technical knowledge.

A person skilled in the art can readily select any appropriate anchor antigen and third (or further) target cell for the design and implementation of the third (or further) antigen-binding moiety according to common general technical knowledge.

In some embodiment, examples of the combinations of the first target cell and the second target cell with which the multispecific antigen-binding molecule trans-activates the Notch signaling pathway in the first target cell, are as follows:
(1) Satellite cells (1st target, for Notch activation) and differentiating myoblast and differentiated myotubule cells (2nd target cell, expressing anchor antigen, e.g., CACNA1S)
(2) Cells secreting pro-fibrotic factors (1st target cell) and any fibroblastic cells (2nd target cell) expressing, e.g., FAP (Fibroblast activated protein) in areas of active tissue remodelling such as tumour stroma or healing wounds. For example, rheumatoid myofibroblast-like synoviocytes, and myofibroblasts.
(3) Activated CD4-T cells (1st target cell) and immune cells (e.g. B-cell, plasma cell, macrophages, monocyte, eosinophil, neutrophil, dendritic cell, mast cell) expressing, e.g., FcgRIIB (2nd target cell) at the inflamed sites.
(4) Pro-tumorigenic cells in tumor microenvironment (1^{st} target cell) which Notch activation would induced anti-cancer effects (e.g. growth inhibition or induce apoptosis) and GPC3 expressing cancer cells (2^{nd} target cell)
(5) Adult intestinal stem cell or crypt base columnar (CBC) cells (1^{st} target cell) and neighboring cells in the intestinal crypts (2^{nd} target cell) (e.g. Paneth cells, +4 cells, transient amplifying cells)
   The potential anchor antigen (2^{nd} target cell) associated with Adult intestinal stem cells or crypt base columnar (CBC) cells can be suitably selected by a skilled person such that the above objective is achieved.

In one such embodiment, any appropriate anchor antigen for the design and implementation of the second (and further) antigen-binding moiety can be selected according to at least one criteria selected from (1) to (5).
1) The spatial expression of the anchor antigen should be restricted to or exclusively expressed by the cell type or tissue of interest to limit systemic exposure and minimize risk of toxicity from Notch activation.
2) The temporal expression of the anchor antigen should be carefully considered. For example, some anchor antigens are only expressed in stem cells and will be lost after commitment to differentiation. Notch activation at different developmental stages also results in different phenotypes in transgenic mice. Early Notch activation leads to embryonic lethality and impaired muscle development. On the contrary, Notch activation in post-natal transgenic mice helps to improve aged muscle and boost muscle regeneration.
3) The anchor antigen should have stable expression on the cell or anchor to the cell surface with slow internalization.
4) The anchor antigen should be uniformly expressed in most cells or tissues of interest with low heterogeneity to minimize uneven activation of Notch signalling.
5) The anchor antigen should be expressed at sufficient levels even in pathological conditions and ensure sufficient retention of bi-specific Notch agonist antibody.

In some embodiments, the present disclosure provides a method of screening for an anchor antigen for a second antigen-binding moiety of a multispecific antigen-binding molecule of the present disclosure, where the method comprises:
(i) assessing whether a candidate anchor antigen satisfies at least one criteria selected from 1) to 5) described above; and
(ii) selecting the anchor antigen for the second antigen-binding moiety if at least one criteria is satisfied.

In some embodiments, the present disclosure provides a method of producing a multispecific antigen-binding molecule which comprises a first antigen-binding moiety and a second antigen-binding moiety, where the method comprises:
(i) assessing whether a candidate anchor antigen satisfies at least one criteria selected from 1) to 5) described above; and
(ii) selecting the anchor antigen for the second antigen-binding moiety if the at least one criteria is satisfied;
(iii) preparing a nucleic acid encoding a multispecific antigen-binding molecule comprising the first antigen-binding moiety which specifically binds to a Notch receptor on a first target cell, and the second antigen-binding moiety which specifically binds to the anchor antigen on a second target cell; and
(iv) expressing the nucleic acid to produce the multispecific antigen-binding molecule, wherein the multispecific antigen-binding molecule activates the Notch signaling pathway in the first target cell when the multispecific antigen-binding molecule is binding to the anchor antigen on the second target cell.

In some embodiments, the present disclosure provides a method of producing a multispecific antigen-binding molecule which comprises a first antigen-binding moiety and a second antigen-binding moiety, where the method comprises:
(i) assessing whether a candidate anchor antigen satisfies at least one criteria selected from 1) to 5) described above; and
(ii) selecting the anchor antigen for the second antigen-binding moiety if the at least one criteria is satisfied;
(iii) preparing a nucleic acid encoding a multispecific antigen-binding molecule comprising the first antigen-binding moiety which specifically binds to a Notch receptor on a first target cell, and the second antigen-binding moiety which specifically binds to the anchor antigen on a second target cell; and
(iv) expressing the nucleic acid to produce the multispecific antigen-binding molecule.

The following list shows candidate anchor antigens or an engineered Fc with preferential binding to an anchor antigen (e.g. Fc gamma RIIB-selective binding technology and Fc gamma RIIB).

| | Subcellular | | |
|---|---|---|---|
| Anchor antigen | localization | Tissue/cell expression | Relevant pathology |
| Calcium Voltage-Gated | Membrane | Skeletal muscle | Muscular |
| Channel Subunit | | | dystrophic |
| Alpha1 S (CACNA1S) | | | conditions |
| Fibroblast activation | Membrane | Activated fibroblast | Tissue fibrosis |
| protein (FAP) | | | |
| FcγRIIB (CD32B) | Membrane | circulating B lymphocytes, | Autoimmune |
| | | monocytes, neutrophils, | diseases |
| | | myeloid dendritic cells | (e.g. SLE, RA and |
| | | (DCs) | MS) |

| List of extracellular proteins as anchor antigen candidates | Tissue |
|---|---|
| Excitatory amino acid transporter 1 | Brain |
| Glutamate [NMDA] receptor subunit zeta 1 | Brain |
| Immunoglobulin superfamily, member 8 | Brain |
| Neuronal cell adhesion molecule | Brain |
| 10 days neonate cortex cDNA, RIKEN library, clone: A830029E02 product: | Brain |
| weakly similar to BK134P22.1 | |
| N-CAM 180 of Neural cell adhesion molecule 1, 180 kDa isoform | Brain |
| Sodium/potassium-transporting ATPase beta-2 chain | Brain |
| DSD-1-proteoglycan | Brain |
| Adult male testis cDNA, synaptic vesicle glycoprotein 2 b | Brain |
| Hepatocyte cell adHesion molecule | Brain |
| Solute carrier family 12 member 5 | Brain |
| Contactin-associated protein-like 2 | Brain |
| Adult male brain UNDEFINED_CELL_LINE cDNA, Proton myo-inositol | Brain |
| transporter homolog | |
| LOC237403 protein | Brain |
| Neurofascin | Brain |
| Contactin-associated protein 1 | Brain |
| Splice Isoform 1 of Chondroitin sulfate proteoglycan 5 | Brain |
| Visual cortex cDNA, RIKEN library, clone: K530020M04 | Brain |
| product: dipeptidylpeptidase 6, full insert sequence | |
| Sodium channel beta-1 subunit precursor | Brain |
| Niemann-Pick C1-like protein 1 | Intestine |
| Oligopeptide transporter, small intestine isoform | Intestine |
| Angiotensin-converting enzyme 2 | Intestine |
| Adult male colon cDNA, RIKEN full-length enriched library, membrane-bound | Intestine |
| aminopeptidase P | |
| NOD-derived CD11c +ve dendritic cells cDNA, hypothetical protein | Intestine |
| 4 days neonate male adipose cDNA, N-acylsphingosine amidohydrolase 2 | Intestine |
| Oligopeptide transporter, small intestine isoform | Intestine |
| Calcium activated chloride channel | Intestine |
| N-acetylated-alpha-linked acidic dipeptidase-like protein | Intestine |
| Tumor necrosis factor receptor superfamily member 13C | Spleen |
| Cannabinoid receptor 2 | Spleen |
| Splice Isoform 1 of B-cell receptor CD22 | Spleen |
| Semaphorin-4D | Spleen |
| Thrombospondin 1 | Spleen |
| Osteoclast-like cell cDNA, granulin | Spleen |
| NOD-derived CD11c +ve dendritic cells cDNA, hypothetical Phospholipase | Spleen |
| D/Transphosphatidylase | |
| L-selectin | Spleen |
| Bone marrow macrophage cDNA, solute carrier family 30 | Spleen |
| B-cell differentiation antigen CD72 | Spleen |
| Transmembrane glycoprotein NMB | Spleen |
| Class II histocompatibility antigen, M beta 1 chain | Spleen |
| Splice Isoform 2 of Sialoadhesin | Spleen |
| Myeloperoxidase | Spleen |
| Leukocyte surface antigen CD53 | Spleen |
| CD180 antigen | Spleen |
| Receptor-type tyrosine-protein phosphatase eta | Spleen |
| Toll-like receptor 9 | Spleen |
| Complement receptor type 2 precursor | Spleen |
| Beta-microseminoprotein | Prostate |
| Adult male urinary bladder cDNA, hypothetical Kazal-type serine protease | Prostate |
| inhibitor domain containing protein | |
| Putative polypeptide N-acetylgalactosaminyltransferase-like protein 4 | Prostate |
| Carcinoembryonic antigen-related cell adhesion molecule 10 | Prostate |
| Adult male tongue cDNA, hypothetical protein | Prostate |
| Seminal vesicle antigen | Prostate |
| Adult male urinary bladder cDNA, weakly similar to LYSOZYME C, TYPE M | Prostate |
| Beta-defensin 50 | Prostate |
| Sodium/bile acid cotransporter | Liver |
| Asialoglycoprotein receptor major subunit | Liver |
| Similar to Rattus norvegicus putative integral membrane transport protein | Liver |
| SLC10A5 | Liver |
| Adult male testis cDNA, similar to PUTATIVE METALLOPEPTIDASE | Testis |
| Zona pellucida sperm-binding protein 3 receptor | Testis |
| Testis-specific protein TES101RP | Testis |
| Dickkopf-like protein 1 | Testis |
| Oviduct-specific glycoprotein | Ovary |
| Procollagen-lysine, 2-oxoglutarate 5-dioxygenase 2 | Ovary |
| Cathepsin L | Ovary |
| Renal sodium-dependent phosphate transport protein 2 | Kidney |
| Gamma-glutamyltranspeptidase 1 | Kidney |
| Splice Isoform 4 of Ssodium- and chloride-dependent transporter XTRP2 | Kidney |
| PREDICTED: similar to low density lipoprotein receptor-related protein 2 | Kidney |
| EP1 | Stomach |
| Potassium-transporting ATPase beta chain | Stomach |
| Secreted gel-forming mucin | Stomach |
| MUC6 | Stomach |
| Lymphocyte antigen 6 complex locus G6C protein | Epidermis |
| Sodium-dependent noradrenaline transporter | Epidermis |
| Solute carrier family 2 (facilitated glucoSe tranSporter), member 4 | Heart |
| Histidine-rich calcium-binding protein | Heart |
| Cadherin-13 | Heart |

In one aspect, the present disclosure provides a multispecific antigen-binding molecule comprising:
(i) a first antigen-binding moiety which specifically binds to an antigen on a tissue stem cell, and
(ii) a second antigen-binding moiety which specifically binds to an anchor antigen on a second target cell,
   wherein the tissue stem cell and the second target cell are different cells, and the multispecific antigen-binding molecule trans-activates the Notch signaling pathway in the tissue stem cell.

In one aspect, the feature, “the multispecific antigen-binding molecule trans-activates the Notch signaling pathway in the tissue stem cell” is alternatively referred to as follows: “the multispecific antigen-binding molecule activates the Notch signaling pathway in the tissue stem cell when (or only when) the multispecific antigen-binding molecule is binding to the anchor antigen on the second target cell. Preferably, the anchor antigen on the second target cell is not expressed (or not significantly/substantially/specifically expressed) in the tissue stem cell.

In one aspect, the present disclosure provides a multispecific antigen-binding molecule comprising:
(i) a first antigen-binding moiety which specifically binds to an antigen on a satellite cell, and
(ii) a second antigen-binding moiety which specifically binds to an anchor antigen on a second target cell,
   wherein the satellite cell and the second target cell are different cells, and the multispecific antigen-binding molecule trans-activates the Notch signaling pathway in the satellite cell. In one aspect, the feature, “the multispecific antigen-binding molecule trans-activates the Notch signaling pathway in the satellite cell” is alternatively referred to as follows: “the multispecific antigen-binding molecule activates the Notch signaling pathway in the satellite cell when (or only when) the multispecific antigen-binding molecule is binding to the anchor antigen on the second target cell. Preferably, the anchor antigen on the second target cell is not expressed (or not significantly/substantially/specifically expressed) in the satellite cell.

### Antigen-Binding Domain

The term “antigen-binding domain” refers to the part of an antibody that comprises the area which specifically binds to and is complementary to part or all of an antigen. An antigen-binding domain may be provided by, for example, one or more antibody variable domains (also called antibody variable regions). Preferably, the antigen-binding domains contain both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F (ab′)2”. An antigen-binding domain may also be provided by single-domain antibodies.

### Single-Domain Antibody

In the present specification, the term “single-domain antibody” is not limited by its structure as long as the domain can exert antigen binding activity by itself. It is known that a general antibody, for example, an IgG antibody, exhibits antigen binding activity in a state where a variable region is formed by the pairing of VH and VL, whereas the own domain structure of the single-domain antibody can exert antigen binding activity by itself without pairing with another domain. Usually, the single-domain antibody has a relatively low molecular weight and exists in the form of a monomer.

Examples of the single-domain antibody include, but are not limited to, antigen-binding molecules congenitally lacking a light chain, such as VHH of an animal of the family Camelidae and shark VNAR, and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present invention, one single-domain antibody has three CDRs (CDR1, CDR2 and CDR3).

The single-domain antibody can be obtained from an animal capable of producing the single-domain antibody or by the immunization of the animal capable of producing the single-domain antibody. Examples of the animal capable of producing the single-domain antibody include, but are not limited to, animals of the family Camelidae, and transgenic animals harboring a gene capable of raising the single-domain antibody. The animals of the family Camelidae include camels, lamas, alpacas, one-hump camels and guanacos, etc. Examples of the transgenic animals harboring a gene capable of raising the single-domain antibody include, but are not limited to, transgenic animals described in International Publication No. WO2015/143414 and U.S. Patent Publication No. US2011/0123527 A1. The framework sequences of the single-domain antibody obtained from the animal may be converted to human germline sequences or sequences similar thereto to obtain a humanized single-domain antibody. The humanized single-domain antibody (e.g., humanized VHH) is also one embodiment of the single-domain antibody of the present invention.

Alternatively, the single-domain antibody can be obtained by ELISA, panning, or the like from a polypeptide library containing single-domain antibodies. Examples of the polypeptide library containing single-domain antibodies include, but are not limited to, naive antibody libraries obtained from various animals or humans (e.g., Methods in Molecular Biology 2012 911 (65-78); and Biochimica et Biophysica Acta—Proteins and Proteomics 2006 1764: 8 (1307-1319)), antibody libraries obtained by the immunization of various animals (e.g., Journal of Applied Microbiology 2014 117: 2 (528-536)), and synthetic antibody libraries prepared from antibody genes of various animals or humans (e.g., Journal of Biomolecular Screening 2016 21: 1 (35-43); Journal of Biological Chemistry 2016 291:24 (12641-12657); and AIDS 2016 30: 11 (1691-1701)).

### Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### HVR or CDR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Hypervariable regions (HVRs) are also referred to as “complementarity determining regions” (CDRs), and these terms are used herein interchangeably in reference to portions of the variable region that form the antigen binding regions. Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 are also mentioned as “H-CDR1”, “H-CDR2”, “H-CDR3”, “L-CDR1”, “L-CDR2”, and “L-CDR3”, respectively.

### Fab Molecule

A “Fab molecule” refers to a protein consisting of the VH and CH1 domain of the heavy chain (the “Fab heavy chain”) and the VL and CL domain of the light chain (the “Fab light chain”) of an immunoglobulin.

### Fused

By “fused” is meant that the components (e.g. a Fab molecule and an Fc domain subunit) are linked by peptide bonds, either directly or via one or more peptide linkers.

### “Crossover” Fab

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

### “Conventional” Fab

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL). The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called alpha (IgA), delta (IgD), epsilon (IgE), gamma (IgG), or mu (IgM), some of which may be further divided into subtypes, e.g. gamma 1 (IgG1), gamma 2 (IgG2), gamma 3 (IgG3), gamma 4 (IgG4), alpha 1 (IgA1) and alpha 2 (IgA2). The light chain of an immunoglobulin may be assigned to one of two types, called kappa and lambda, based on the amino acid sequence of its constant domain. An immunoglobulin essentially consists of two Fab molecules and an Fc domain, linked via the immunoglobulin hinge region.

### Affinity/Avidity

“Affinity” refers to the strength of the sum total of noncovalent interactions between a single binding site of a molecule (e.g., an antigen-binding molecule or antibody) and its binding partner (e.g., an antigen). Unless indicated otherwise, as used herein, “binding affinity” refers to intrinsic binding affinity which reflects a 1:1 interaction between members of a binding pair (e.g., antigen-binding molecule and antigen, or antibody and antigen). The affinity of a molecule X for its partner Y can generally be represented by the dissociation constant (KD), which is the ratio of dissociation and association rate constants (koff and kon, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

The structure of the antigen-binding domain of an antibody that binds to the epitope is called paratope. The paratope stably binds to the epitope through a hydrogen bond, electrostatic force, van der Waals' forces, a hydrophobic bond, or the like acting between the epitope and the paratope. This binding force between the epitope and the paratope is called “affinity” (see also above). The total binding force when a plurality of antigen binding domains bind to a plurality of antigens is called “avidity”. The affinity works synergistically when, for example, an antibody comprising a plurality of antigen binding domains (i.e., a polyvalent antibody) bind to a plurality of epitopes, and the avidity may be higher than the affinity.

### Methods to Determine Affinity

In certain embodiments, the antigen-binding molecule or antibody provided herein has a dissociation constant (KD) of 1 micromolar (micro M) or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10⁻⁸ M or less, 10⁻⁸ M to 10⁻¹³ M, 10⁻⁹ M to 10⁻¹³ M) for its antigen. In certain embodiments, the KD value of the antibody/antigen-binding molecule for Notch receptor or anchor antigen falls within the range of 1-40, 1-50, 1-70, 1-80, 30-50, 30-70, 30-80, 40-70, 40-80, or 60-80 nM.

In one embodiment, KD is measured by a radiolabeled antigen-binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 microgram (micro g)/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 microliter (micro 1)/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, NJ) is performed at 25 degrees C. with immobilized antigen CM5 chips at approximately 10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (approximately 0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

According to the methods for measuring the affinity of the antigen-binding molecule or the antibody described above, persons skilled in art can carry out affinity measurement for other antigen-binding molecules or antibodies, towards various kind of antigens.

### Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

Unless otherwise indicated, amino acid residues in the light chain constant region are numbered herein according to Kabat et al., and numbering of amino acid residues in the heavy chain constant region is according to the EU numbering system, also called the EU index, as described in Kabat et al., Sequences of Proteins of Immunological Interest, 5th Ed. Public Health Service, National Institutes of Health, Bethesda, M D, 1991.

### Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

### Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

### Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

### Polynucleotide (Nucleic Acid)

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

### Isolated (Nucleic Acid)

An “isolated” nucleic acid molecule is one which has been separated from a component of its natural environment. An isolated nucleic acid molecule further includes a nucleic acid molecule contained in cells that ordinarily contain the nucleic acid molecule, but the nucleic acid molecule is present extrachromosomally or at a chromosomal location that is different from its natural chromosomal location.

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors could be introduced into host cells using virus or electroporation. However, introduction of vectors is not limited to in vitro method. For example, vectors could also be introduced into a subject using in vivo method directly.

### Host Cell

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### Specificity

“Specific” means that a molecule that binds specifically to one or more binding partners does not show any significant binding to molecules other than the partners. Furthermore, “specific” is also used when an antigen-binding site is specific to a particular epitope of multiple epitopes contained in an antigen. If an antigen-binding molecule binds specifically to an antigen, it is also described as “the antigen-binding molecule has/shows specificity to/towards the antigen”. When an epitope bound by an antigen-binding site is contained in multiple different antigens, an antigen-binding molecule containing the antigen-binding site can bind to various antigens that have the epitope.

### Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

### Variable Fragment (Fv)

Herein, the term “variable fragment (Fv)” refers to the minimum unit of an antibody-derived antigen-binding site that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

scFv, Single-Chain Antibody, and Sc(Fv)₂

Herein, the terms “scFv”, “single-chain antibody”, and “sc(Fv)₂” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen-binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

An scFv is an single chain low molecule weight antibody in which VH and VL forming Fv are linked together by a peptide linker (Proc. Natl. Acad. Sci. U.S.A. (1988) 85(16), 5879-5883). VH and VL can be retained in close proximity by the peptide linker. sc(Fv)₂ is a single chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)₂ preferably includes, for example, a bispecific sc(Fv)₂ that recognizes two epitopes present in a single antigen as disclosed in the Journal of Immunology (1994) 152(11), 5368-5374. sc(Fv)₂ can be produced by methods known to those skilled in the art. For example, sc(Fv)₂ can be produced by linking scFv by a linker such as a peptide linker.

Herein, an sc(Fv)₂ includes two VH units and two VL units which are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below.

[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

The molecular form of sc(Fv)₂ is also described in detail in WO 2006/132352. According to these descriptions, those skilled in the art can appropriately prepare desired sc(Fv)₂ to produce the polypeptide complexes disclosed herein.

Furthermore, the antigen-binding molecules or antibodies of the present disclosure may be conjugated with a carrier polymer such as PEG or an organic compound such as an anticancer agent. Alternatively, a sugar chain addition sequence is preferably inserted into the antigen-binding molecules or antibodies such that the sugar chain produces a desired effect.

The linkers to be used for linking the variable regions of an antibody comprise arbitrary peptide linkers that can be introduced by genetic engineering, synthetic linkers, and linkers disclosed in, for example, Protein Engineering, 9(3), 299-305, 1996. However, peptide linkers are preferred in the present disclosure. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)₂ contains three peptide linkers, their length may be all the same or different.

For example, such peptide linkers include:

Ser,
Gly-Ser,
Gly-Gly-Ser,
Ser-Gly-Gly,
(SEQ ID NO: 9)
Gly-Gly-Gly-Ser,
(SEQ ID NO: 10)
Ser-Gly-Gly-Gly,
(SEQ ID NO: 11)
Gly-Gly-Gly-Gly-Ser,
(SEQ ID NO: 12)
Ser-Gly-Gly-Gly-Gly,
(SEQ ID NO: 13)
Gly-Gly-Gly-Gly-Gly-Ser,
(SEQ ID NO: 14)
Ser-Gly-Gly-Gly-Gly-Gly,
(SEQ ID NO: 15)
Gly-Gly-Gly-Gly-Gly-Gly-Ser,
(SEQ ID NO: 16)
Ser-Gly-Gly-Gly-Gly-Gly-Gly,
(Gly-Gly-Gly-Gly-Ser (SEQ ID NO: 11))n,
and
(Ser-Gly-Gly-Gly-Gly (SEQ ID NO: 12))n,

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:
N-hydroxy succinimide (NHS),
disuccinimidyl suberate (DSS),
bis(sulfosuccinimidyl) suberate (BS3),
dithiobis(succinimidyl propionate) (DSP),
dithiobis(sulfosuccinimidyl propionate) (DTSSP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl] sulfone (BSOCOES), and
bis[2-(sulfosuccinimidoxycarbonyloxy)ethyl] sulfone (sulfo-BSOCOES). These crosslinking agents are commercially available.

In general, three linkers are required to link four antibody variable regions together. The linkers to be used may be of the same type or different types.

### Fab, F(ab′)₂, and Fab′

“Fab” consists of a single light chain, and a CH1 domain and variable region from a single heavy chain. The heavy chain of Fab molecule cannot form disulfide bonds with another heavy chain molecule.

“F(ab′)₂” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)₂” consists of two light chains and two heavy chains comprising the constant region of a CH1 domain and a portion of CH2 domains so that disulfide bonds are formed between the two heavy chains. The F(ab′)₂ disclosed herein can be preferably produced as follows. A whole monoclonal antibody or such comprising a desired antigen-binding site is partially digested with a protease such as pepsin; and Fc fragments are removed by adsorption onto a Protein A column. The protease is not particularly limited, as long as it can cleave the whole antibody in a selective manner to produce F(ab′)₂ under an appropriate setup enzyme reaction condition such as pH. Such proteases include, for example, pepsin and ficin.

### Fc Region

The term “Fc region” or “Fc domain” refers to a region comprising a fragment consisting of a hinge or a portion thereof and CH2 and CH3 domains in an antibody molecule. The Fc region of IgG class means, but is not limited to, a region from, for example, cysteine 226 (EU numbering (also referred to as EU index herein)) to the C terminus or proline 230 (EU numbering) to the C terminus. The Fc region can be preferably obtained by the partial digestion of, for example, an IgG1, IgG2, IgG3, or IgG4 monoclonal antibody with a proteolytic enzyme such as pepsin followed by the re-elution of a fraction adsorbed on a protein A column or a protein G column. Such a proteolytic enzyme is not particularly limited as long as the enzyme is capable of digesting a whole antibody to restrictively form Fab or F(ab′)2 under appropriately set reaction conditions (e.g., pH) of the enzyme. Examples thereof can include pepsin and papain.

An Fc region derived from, for example, naturally occurring IgG can be used as the “Fc region” of the present disclosure. In this context, the naturally occurring IgG means a polypeptide that contains an amino acid sequence identical to that of IgG found in nature and belongs to a class of an antibody substantially encoded by an immunoglobulin gamma gene. The naturally occurring human IgG means, for example, naturally occurring human IgG1, naturally occurring human IgG2, naturally occurring human IgG3, or naturally occurring human IgG4. The naturally occurring IgG also includes variants or the like spontaneously derived therefrom. A plurality of allotype sequences based on gene polymorphism are described as the constant regions of human IgG1, human IgG2, human IgG3, and human IgG4 antibodies in Sequences of proteins of immunological interest, NIH Publication No. 91-3242, any of which can be used in the present disclosure. Particularly, the sequence of human IgG1 may have DEL or EEM as an amino acid sequence of EU numbering positions 356 to 358.

In certain embodiments, one or more amino acid substitutions may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid substitution at one or more amino acid positions. For instance, the heavy chain constant region of human IgG1, human IgG2, human IgG3, and human IgG4 are shown in SEQ ID NOs: 18 to 21, respectively. For instance, the Fc region of human IgG1, human IgG2, human IgG3, and human IgG4 are shown as a partial sequence of SEQ ID NOs: 18 to 21.

In some embodiments, the Fc domain of the multispecific antigen-binding molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the multispecific antigen-binding molecule described herein comprises not more than one Fc domain.

In one embodiment described herein, the Fc domain of the multispecific-antigen binding molecule is an IgG Fc domain. In a particular embodiment, the Fc domain is an IgG1 Fc domain. In another embodiment, the Fc domain is an IgG1 Fc domain. In a further particular embodiment, the Fc domain is a human IgG1 Fc region.

In one aspect, the present disclosure provides a multispecific antigen-binding molecule further comprising
(i) an Fc domain which exhibits reduced binding affinity to human Fc gamma receptor, as compared to a native human IgG1 Fc domain,
   wherein the Fc domain is composed of a first Fc-region subunit and a second Fc-region subunit that are capable of stable association.

In one aspect, the present disclosure provides a multispecific antigen-binding molecule further comprising
(i) an Fc domain which exhibits reduced binding affinity to human Fc gamma receptor, as compared to a native human IgG1 Fc domain,
   wherein the Fc domain comprises (e1) or (e2) below:
(e1) the first Fc-region subunit comprising Cys at position 349, Ser at position 366, Ala at position 368 and Val at position 407, and the second Fc-region comprising Cys at position 354 and Trp at position 366;
(e2) the first Fc-region subunit comprising Glu at position 439, and the second Fc-region comprising Lys at position 356;
   wherein the amino acid positions are numbered according to EU index.

In one aspect, the present disclosure provides a multispecific antigen-binding molecule further comprising
(i) an Fc domain which exhibits reduced binding affinity to human Fc gamma receptor, as compared to a native human IgG1 Fc domain,
   wherein the first and/or the second Fc-region subunit comprised in the Fc domain comprises (f1) or (f2) below:
(f1) Ala at position 234 and Ala at position 235;
(f2) Ala at position 234, Ala at position 235 and Ala at position 297; wherein the amino acid positions are numbered according to EU index.

In one aspect, the present disclosure provides a multispecific antigen-binding molecule further comprising
(i) an Fc domain which exhibits reduced binding affinity to human Fc gamma receptor, as compared to a native human IgG1 Fc domain,
   wherein the Fc domain further exhibits stronger FcRn binding affinity to human FcRn, as compared to a native human IgG1 Fc domain.

In one aspect, the present disclosure provides a multispecific antigen-binding molecule further comprising
(i) an Fc domain which exhibits reduced binding affinity to human Fc gamma receptor, as compared to a native human IgG1 Fc domain,
   wherein the first and/or Fc region subunit comprised in the Fc domain comprises Leu at position 428, Ala at position 434, Arg at position 438, and Glu at position 440, wherein the amino acid positions are numbered according to EU index.
   Fc Region with a Reduced Fc Receptor (Fc Gamma Receptor)-Binding Activity

In certain embodiments, the Fc domain of the multispecific antigen-binding molecules described herein exhibits reduced binding affinity to an Fc receptor, as compared to a native IgG1 Fc domain. In one such embodiment the Fc domain (or the multispecific antigen-binding molecule comprising said Fc domain) exhibits less than 50%, preferably less than 20%, more preferably less than 10% and most preferably less than 5% of the binding affinity to an Fc receptor, as compared to a native IgG1 Fc domain (or a multispecific antigen-binding molecule comprising a native IgG1 Fc domain). In one embodiment, the Fc domain (or the multispecific antigen-binding molecule comprising said Fc domain) does not substantially bind to an Fc receptor. In a particular embodiment, the Fc receptor is an Fc gamma receptor. In one embodiment the Fc receptor is a human Fc receptor. In one embodiment, the Fc receptor is an activating Fc receptor. In a specific embodiment the Fc receptor is an activating human Fc gamma receptor, more specifically human Fc gamma RIIIa, Fc gamma RI or Fc gamma RIIa, most specifically human Fc gamma RIIIa.

In certain embodiments, the Fc domain of the multispecific antigen-binding molecule comprises one or more amino acid mutation that reduces the binding affinity of the Fc domain to an Fc receptor. Typically, the same one or more amino acid mutation is present in each of the two subunits of the Fc domain. In one embodiment the amino acid mutation reduces the binding affinity of the Fc domain to an Fc receptor. In one embodiment, the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the multispecific antigen-binding molecule comprising an engineered Fc domain exhibits less than 20%, particularly less than 10%, more particularly less than 5% of the binding affinity to an Fc receptor as compared to a multispecific antigen-binding molecule comprising a non-engineered Fc domain. In a particular embodiment the Fc receptor is an Fc gamma receptor. In some embodiments, the Fc receptor is a human Fc receptor. In some embodiments the Fc receptor is an activating Fc receptor. In a specific embodiment, the Fc receptor is an activating human Fc gamma receptor, more specifically human Fc gamma RIIIa, Fc gamma RI or Fc gamma RIIa, most specifically human Fc gamma RIIIa. Preferably, binding to each of these receptors is reduced.

In one embodiment, the amino acid mutation that reduces the binding affinity of the Fc domain to an Fc receptor is an amino acid substitution. In one embodiment, the Fc domain comprises an amino acid substitution at a position selected from the group of E233, L234, L235, N297, P331 and P329. In a more specific embodiment, the Fc domain comprises an amino acid substitution at a position selected from the group of L234, L235 and P329. In some embodiments, the Fc domain comprises the amino acid substitutions L234A and L235A. In one such embodiment, the Fc domain is an IgG1 Fc domain, particularly a human IgG1 Fc domain. In one embodiment, the Fc domain comprises an amino acid substitution at position P329. In a more specific embodiment, the amino acid substitution is P329A or P329G, particularly P329G. In one embodiment the Fc domain comprises an amino acid substitution at position P329 and a further amino acid substitution at a position selected from E233, L234, L235, N297 and P331. In a more specific embodiment, the further amino acid substitution is E233P, L234A, L235A, L235E, N297A, N297D or P331S. In particular embodiments, the Fc domain comprises amino acid substitutions at positions P329, L234 and L235. In more particular embodiments the Fc domain comprises the amino acid mutations L234A, L235A and P329G (“P329G LALA”). In one such embodiment, the Fc domain is an IgG1 Fc domain, particularly a human IgG1 Fc domain. The “P329G LALA” combination of amino acid substitutions almost completely abolishes Fc gamma receptor (as well as complement) binding of a human IgG1 Fc domain, as described in PCT publication no. WO 2012/130831. WO 2012/130831 also describes methods of preparing such mutant Fc domains and methods for determining its properties such as Fc receptor binding or effector functions.

IgG4 antibodies exhibit reduced binding affinity to Fc receptors and reduced effector functions as compared to IgG1 antibodies. Hence, in some embodiments, the Fc domain of the T cell activating bispecific antigen binding molecules described herein is an IgG4 Fc domain, particularly a human IgG4 Fc domain. In one embodiment, the IgG4 Fc domain comprises amino acid substitutions at position S228, specifically the amino acid substitution S228P. To further reduce its binding affinity to an Fc receptor and/or its effector function, in one embodiment the IgG4 Fc domain comprises an amino acid substitution at position L235, specifically the amino acid substitution L235E. In another embodiment, the IgG4 Fc domain comprises an amino acid substitution at position P329, specifically the amino acid substitution P329G. In a particular embodiment, the IgG4 Fc domain comprises amino acid substitutions at positions S228, L235 and P329, specifically amino acid substitutions S228P, L235E and P329G. Such IgG4 Fc domain mutants and their Fc gamma receptor binding properties are described in PCT publication no. WO 2012/130831.

In certain embodiments, N-glycosylation of the Fc domain has been eliminated. In one such embodiment, the Fc domain comprises an amino acid mutation at position N297, particularly an amino acid substitution replacing asparagine by alanine (N297A) or aspartic acid (N297D).

In a particular preferred embodiment, the Fc domain exhibiting reduced binding affinity to an Fc receptor, as compared to a native IgG1 Fc domain, is a human IgG1 Fc domain comprising the amino acid substitutions L234A, L235A and N297A.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing Fc gamma IIIa receptor.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

### Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma RIb and Fc gamma RIc; Fc gamma RII (CD32) including isoforms Fc gamma RIIa (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIIIa (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RII (CD32), Fc gamma RIII (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fc gamma RI (CD64), Fc gamma RIIA (CD32), Fc gamma RIIB (CD32), Fc gamma RIIIA (CD16), and/or Fc gamma RIIIB (CD16). The polynucleotide sequence and amino acid sequence of Fc gamma RI are shown in RefSeq accession number NM_000566.3 and RefSeq accession number NP_000557.1, respectively; the polynucleotide sequence and amino acid sequence of Fc gamma RIIA are shown in RefSeq accession number BC020823.1 and RefSeq accession number AAH20823.1, respectively; the polynucleotide sequence and amino acid sequence of Fc gamma RIIB are shown in RefSeq accession number BC146678.1 and RefSeq accession number AAI46679.1, respectively; the polynucleotide sequence and amino acid sequence of Fc gamma RIIIA are shown in RefSeq accession number BC033678.1 and RefSeq accession number AAH33678.1, respectively; and the polynucleotide sequence and amino acid sequence of Fc gamma RIIIB are shown in RefSeq accession number BC128562.1 and RefSeq accession number AAI28563.1, respectively. Whether an Fc gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fc gamma receptor, Fc alpha receptor, Fc beta receptor, FcRn, C1q, and C3, mannan-binding lectin, mannose receptor, Staphylococcus Protein A, Staphylococcus Protein G, and viral Fc gamma receptors. The Fc ligands also include Fc receptor homologs (FcRH) (Davis et al., (2002) Immunological Reviews 190, 123-136), which are a family of Fc receptors homologous to Fc gamma receptor. The Fc ligands also include unidentified molecules that bind to Fc.

### Fc Gamma Receptor-Binding Activity

The impaired binding activity of Fc domain to any of the Fc gamma receptors Fc gamma RI, Fc gamma RIIA, Fc gamma RIIB, Fc gamma RIIIA, and/or Fc gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fc domain, inducing a signal of 520 to 620 nm as a result. The antigen-binding molecule or antibody having a non-tagged mutant Fc domain competes with the antigen-binding molecule or antibody comprising a wild-type Fc domain for the interaction with Fc gamma receptor. The relative binding affinity can be determined by quantifying the reduction of fluorescence as a result of competition. Methods for biotinylating the antigen-binding molecules or antibodies such as antibodies using Sulfo-NHS-biotin or the like are known. Appropriate methods for adding the GST tag to an Fc gamma receptor include methods that involve fusing polypeptides encoding Fc gamma receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE methods. Examples of such inhibition assay are described in Proc. Natl. Acad. Sci. USA (2006) 103(11), 4005-4010.

Engineered Fc Region (“Fc Region Variant”) that Specifically Binds to Fc Gamma RIIb

In one aspect, the multispecific antigen-binding molecule of the present disclosure comprises a second antigen-binding moiety comprising an engineered Fc region (“Fc region variant”) which specifically binds to an anchor antigen on a second target cell.

In one aspect, the multispecific antigen-binding molecule of the present disclosure comprises a second antigen-binding moiety comprising an engineered Fc region (“Fc region variant”) which specifically binds to FcgRIIB. In one embodiment, the engineered Fc region which specifically binds to FcgRIIB can lower binding activities to all activating Fc gamma Rs, in particular Fc gamma RIIa (R type), while maintaining Fc gamma RIIb-binding activity, when compared to a polypeptide containing a native IgG antibody Fc region.

More specifically, the invention provides Fc region variants comprising an amino acid sequence in which the amino acid alteration at position 238 according to EU numbering is combined with other specific amino acid alterations.

Furthermore, the present invention provides methods of introducing the amino acid alterations into an Fc region for decreasing its binding activities to all activating Fc gamma Rs, in particular Fc gamma RIIa (R type), while maintaining its Fc gamma RIIb-binding activity in comparison to those of a polypeptide containing a native IgG antibody Fc region.

In some embodiments, the engineered Fc region (“Fc region variant”) that specifically binds to Fc gamma RIIb of the present disclosure comprises an Fc region variant which comprises amino acid alterations that combine an alteration of the amino acid at position 238 according to EU numbering to another amino acid with alteration of any one of the amino acids of (a) to (k) below to another amino acid in the Fc region of human IgG (IgG1, IgG2, IgG3, and IgG4). Introducing the alterations into an Fc region can provide a polypeptide comprising an Fc region variant with decreased binding activities to all activating Fc gamma Rs, in particular Fc gamma RIIa (R type), while maintaining the Fc gamma RIIb-binding activity as compared to those of a polypeptide comprising the Fc region of a native IgG:
(a) amino acid at position 235 of the Fc region according to EU numbering;
(b) amino acid at position 237 of the Fc region according to EU numbering;
(c) amino acid at position 241 of the Fc region according to EU numbering;
(d) amino acid at position 268 of the Fc region according to EU numbering;
(e) amino acid at position 295 of the Fc region according to EU numbering;
(f) amino acid at position 296 of the Fc region according to EU numbering;
(g) amino acid at position 298 of the Fc region according to EU numbering;
(h) amino acid at position 323 of the Fc region according to EU numbering;
(i) amino acid at position 324 of the Fc region according to EU numbering;
(j) amino acid at position 330 of the Fc region according to EU numbering; and
(k) at least two amino acids selected from (a) to (j).
   Methods for Producing an Antibody with Desired Binding Activity

Methods for producing an antibody with desired binding activity are known to those skilled in the art. Below is an example that describes a method for producing an antibody (anti-Notch Receptor antibody) that binds to Notch receptor.

Antibodies that bind to an anchor antigen on a second target cell or a third target cell can also be produced according to the example described below.

Anti-Notch receptor antibodies can be obtained as polyclonal or monoclonal antibodies using known methods. The anti-Notch receptor antibodies preferably produced are monoclonal antibodies derived from mammals. Such mammal-derived monoclonal antibodies include antibodies produced by hybridomas or host cells transformed with an expression vector carrying an antibody gene by genetic engineering techniques.

Monoclonal antibody-producing hybridomas can be produced using known techniques, for example, as described below. Specifically, mammals are immunized by conventional immunization methods using a Notch receptor protein as a sensitizing antigen. Resulting immune cells are fused with known parental cells by conventional cell fusion methods. Then, hybridomas producing an anti-Notch receptor antibody can be selected by screening for monoclonal antibody-producing cells using conventional screening methods.

Specifically, monoclonal antibodies are prepared as mentioned below. First, the Notch receptor gene whose nucleotide sequence is disclosed in RefSeq Accession Nos. for Notch1 (NP_060087.3 or P46531), Notch2 (NP_077719.2 (isoform 1) or NP_001186930.1 (isoform 2)), Notch3 (NP_000426.2), or Notch4 (NP_004548.3 or Q99466) can be expressed to produce the Notch receptor protein shown as follows.

(The amino acid sequence of human Notch receptor1: Genbank Accession No. P46531, human Notch receptor2: Genbank Accession No. AAH71562.2, human Notch receptor3: Genbank Accession No. AAB91371.1, human Notch receptor4: Genbank Accession No. AAC63097.1.)

Those proteins will be used as a sensitizing antigen for antibody preparation. Alternatively, a nucleotide encoding the extracellular domain (ECD) of Notch receptor can be expressed to produce a Notch receptor ECD-containing protein. That is, a gene sequence encoding full-length Notch receptor or Notch receptor ECD is inserted into a known expression vector, and appropriate host cells are transformed with this vector. The extracellular domain of Notch receptor could be used. The desired human full-length Notch receptor or Notch receptor ECD protein is purified from the host cells or their culture supernatants by known methods. Alternatively, it is possible to use a purified natural Notch receptor protein as a sensitizing antigen.

The purified full-length Notch receptor or Notch receptor ECD protein can be used as a sensitizing antigen for use in immunization of mammals. Partial peptides of full-length Notch receptor or Notch receptor ECD can also be used as sensitizing antigens. In this case, the partial peptides may also be obtained by chemical synthesis from the human Notch receptor amino acid sequence. Furthermore, they may also be obtained by incorporating a portion of the Notch receptor gene into an expression vector and expressing it. Moreover, they may also be obtained by degrading the Notch receptor protein using proteases, but the region and size of the Notch receptor peptide used as the partial peptide are not particularly limited to a special embodiment.

For sensitizing antigen, alternatively it is possible to use a fusion protein prepared by fusing a desired partial polypeptide or peptide of the full-length Notch receptor or Notch receptor ECD protein with a different polypeptide. For example, antibody Fc fragments and peptide tags are preferably used to produce fusion proteins to be used as sensitizing antigens. Vectors for expression of such fusion proteins can be constructed by fusing in frame genes encoding two or more desired polypeptide fragments and inserting the fusion gene into an expression vector as described above. Methods for producing fusion proteins are described in Molecular Cloning 2nd ed. (Sambrook, J et al., Molecular Cloning 2nd ed., 9.47-9.58 (1989) Cold Spring Harbor Lab. Press). Methods for preparing Notch receptor to be used as a sensitizing antigen, and immunization methods using Notch receptor are also generally known.

There is no particular limitation on the mammals to be immunized with the sensitizing antigen. However, it is preferable to select the mammals by considering their compatibility with the parent cells to be used for cell fusion. In general, rodents such as mice, rats, and hamsters, rabbits, and monkeys are preferably used.

The above animals are immunized with a sensitizing antigen by known methods. Generally performed immunization methods include, for example, intraperitoneal or subcutaneous injection of a sensitizing antigen into mammals. Specifically, a sensitizing antigen is appropriately diluted with PBS (Phosphate-Buffered Saline), physiological saline, or the like. If desired, a conventional adjuvant such as Freund's complete adjuvant is mixed with the antigen, and the mixture is emulsified. Then, the sensitizing antigen is administered to a mammal several times at 4- to 21-day intervals. Appropriate carriers may be used in immunization with the sensitizing antigen. In particular, when a low-molecular-weight partial peptide is used as the sensitizing antigen, it is sometimes desirable to couple the sensitizing antigen peptide to a carrier protein such as albumin or keyhole limpet hemocyanin for immunization.

Alternatively, hybridomas producing a desired antibody can be prepared using DNA immunization as mentioned below. DNA immunization is an immunization method that confers immunostimulation by expressing a sensitizing antigen in an animal immunized as a result of administering a vector DNA constructed to allow expression of an antigen protein-encoding gene in the animal. As compared to conventional immunization methods in which a protein antigen is administered to animals to be immunized, DNA immunization is expected to be superior in that:
immunostimulation can be provided while retaining the structure of a membrane protein such as Notch receptor; and
there is no need to purify the antigen for immunization.

In order to prepare a monoclonal antibody of the present invention using DNA immunization, first, a DNA expressing a Notch receptor protein is administered to an animal to be immunized. The Notch receptor-encoding DNA can be synthesized by known methods such as PCR. The obtained DNA is inserted into an appropriate expression vector, and then this is administered to an animal to be immunized. Preferably used expression vectors include, for example, commercially-available expression vectors such as pcDNA3.1. Vectors can be administered to an organism using conventional methods. For example, DNA immunization is performed by using a gene gun to introduce expression vector-coated gold particles into cells in the body of an animal to be immunized. Antibodies that recognized Notch receptor can also be produced by the methods described in WO 2003/104453.

After immunizing a mammal as described above, an increase in the titer of a Notch receptor-binding antibody is confirmed in the serum. Then, immune cells are collected from the mammal, and then subjected to cell fusion. In particular, splenocytes are preferably used as immune cells.

A mammalian myeloma cell is used as a cell to be fused with the above-mentioned immunocyte. The myeloma cells preferably comprise a suitable selection marker for screening. A selection marker confers characteristics to cells for their survival (or death) under a specific culture condition. Hypoxanthine-guanine phosphoribosyltransferase deficiency (hereinafter abbreviated as HGPRT deficiency) and thymidine kinase deficiency (hereinafter abbreviated as TK deficiency) are known as selection markers. Cells with HGPRT or TK deficiency have hypoxanthine-aminopterin-thymidine sensitivity (hereinafter abbreviated as HAT sensitivity). HAT-sensitive cells cannot synthesize DNA in a HAT selection medium, and are thus killed. However, when the cells are fused with normal cells, they can continue DNA synthesis using the salvage pathway of the normal cells, and therefore they can grow even in the HAT selection medium.

HGPRT-deficient and TK-deficient cells can be selected in a medium containing 6-thioguanine, 8-azaguanine (hereinafter abbreviated as 8AG), or 5′-bromodeoxyuridine, respectively. Normal cells are killed because they incorporate these pyrimidine analogs into their DNA. Meanwhile, cells that are deficient in these enzymes can survive in the selection medium, since they cannot incorporate these pyrimidine analogs. In addition, a selection marker referred to as G418 resistance provided by the neomycin-resistant gene confers resistance to 2-deoxystreptamine antibiotics (gentamycin analogs). Various types of myeloma cells that are suitable for cell fusion are known.

For example, myeloma cells including the following cells can be preferably used:
P3(P3x63Ag8.653) (J. Immunol. (1979) 123 (4), 1548-1550);
P3x63Ag8U.1 (Current Topics in Microbiology and Immunology (1978)81, 1-7);
NS-1 (C. Eur. J. Immunol. (1976)6 (7), 511-519);
MPC-11 (Cell (1976) 8 (3), 405-415);
SP2/0 (Nature (1978) 276 (5685), 269-270);
FO (J. Immunol. Methods (1980) 35 (1-2), 1-21);
S194/5.XX0.BU.1 (J. Exp. Med. (1978) 148 (1), 313-323);
R210 (Nature (1979) 277 (5692), 131-133), etc.

Cell fusions between the immunocytes and myeloma cells are essentially carried out using known methods, for example, a method by Kohler and Milstein et al. (Methods Enzymol. (1981) 73: 3-46).

More specifically, cell fusion can be carried out, for example, in a conventional culture medium in the presence of a cell fusion-promoting agent. The fusion-promoting agents include, for example, polyethylene glycol (PEG) and Sendai virus (HVJ). If required, an auxiliary substance such as dimethyl sulfoxide is also added to improve fusion efficiency.

The ratio of immunocytes to myeloma cells may be determined at one's own discretion, preferably, for example, one myeloma cell for every one to ten immunocytes. Culture media to be used for cell fusions include, for example, media that are suitable for the growth of myeloma cell lines, such as RPMI1640 medium and MEM medium, and other conventional culture medium used for this type of cell culture. In addition, serum supplements such as fetal calf serum (FCS) may be preferably added to the culture medium.

For cell fusion, predetermined amounts of the above immune cells and myeloma cells are mixed well in the above culture medium. Then, a PEG solution (for example, the average molecular weight is about 1,000 to 6,000) prewarmed to about 37 degrees Celsius (C) is added thereto at a concentration of generally 30% to 60% (w/v). This is gently mixed to produce desired fusion cells (hybridomas). Then, an appropriate culture medium mentioned above is gradually added to the cells, and this is repeatedly centrifuged to remove the supernatant. Thus, cell fusion agents and such which are unfavorable to hybridoma growth can be removed.

The hybridomas thus obtained can be selected by culture using a conventional selective medium, for example, HAT medium (a culture medium containing hypoxanthine, aminopterin, and thymidine). Cells other than the desired hybridomas (non-fused cells) can be killed by continuing culture in the above HAT medium for a sufficient period of time. Typically, the period is several days to several weeks. Then, hybridomas producing the desired antibody are screened and singly cloned by conventional limiting dilution methods.

The hybridomas thus obtained can be selected using a selection medium based on the selection marker possessed by the myeloma used for cell fusion. For example, HGPRT- or TK-deficient cells can be selected by culture using the HAT medium (a culture medium containing hypoxanthine, aminopterin, and thymidine). Specifically, when HAT-sensitive myeloma cells are used for cell fusion, cells successfully fused with normal cells can selectively proliferate in the HAT medium. Cells other than the desired hybridomas (non-fused cells) can be killed by continuing culture in the above HAT medium for a sufficient period of time. Specifically, desired hybridomas can be selected by culture for generally several days to several weeks. Then, hybridomas producing the desired antibody are screened and singly cloned by conventional limiting dilution methods.

Desired antibodies can be preferably selected and singly cloned by screening methods based on known antigen/antibody reaction. For example, a Notch receptor-binding monoclonal antibody can bind to Notch receptor expressed on the cell surface. Such a monoclonal antibody can be screened by fluorescence activated cell sorting (FACS). FACS is a system that assesses the binding of an antibody to cell or cell surface by analyzing cells contacted with a fluorescent antibody using laser beam, and measuring the fluorescence emitted from individual cells.

To screen for hybridomas that produce a monoclonal antibody of the present invention by FACS, Notch receptor-expressing cells are first prepared. Cells preferably used for screening are mammalian cells in which Notch receptor is forcedly expressed. As control, the activity of an antibody to bind to cell-surface Notch receptor can be selectively detected using non-transformed mammalian cells as host cells. Specifically, hybridomas producing an anti-Notch receptor monoclonal antibody can be isolated by selecting hybridomas that produce an antibody which binds to cells forced to express Notch receptor, but not to host cells.

Alternatively, the activity of an antibody to bind to immobilized Notch receptor-expressing cells can be assessed based on the principle of ELISA. For example, Notch receptor-expressing cells are immobilized to the wells of an ELISA plate. Culture supernatants of hybridomas are contacted with the immobilized cells in the wells, and antibodies that bind to the immobilized cells are detected. When the monoclonal antibodies are derived from mouse, antibodies bound to the cells can be detected using an anti-mouse immunoglobulin antibody. Hybridomas producing a desired antibody having the antigen-binding ability are selected by the above screening, and they can be cloned by a limiting dilution method or the like.

Monoclonal antibody-producing hybridomas thus prepared can be passaged in a conventional culture medium, and stored in liquid nitrogen for a long period.

The above hybridomas are cultured by a conventional method, and desired monoclonal antibodies can be prepared from the culture supernatants. Alternatively, the hybridomas are administered to and grown in compatible mammals, and monoclonal antibodies are prepared from the ascites. The former method is suitable for preparing antibodies with high purity.

Antibodies encoded by antibody genes that are cloned from antibody-producing cells such as the above hybridomas can also be preferably used. A cloned antibody gene is inserted into an appropriate vector, and this is introduced into a host to express the antibody encoded by the gene. Methods for isolating antibody genes, inserting the genes into vectors, and transforming host cells have already been established, for example, by Vandamme et al. (Eur. J. Biochem. (1990) 192(3), 767-775). Methods for producing recombinant antibodies are also known as described below.

Preferably, the present invention provides nucleic acids that encode a multispecific antigen-binding molecule of the present invention. The present invention also provides a vector into which the nucleic acid encoding the multispecific antigen-binding molecule is introduced, i.e., a vector comprising the nucleic acid. Furthermore, the present invention provides a cell comprising the nucleic acid or the vector. The present invention also provides a method for producing the multispecific antigen-binding molecule by culturing the cell. The present invention further provides multispecific antigen-binding molecules produced by the method.

For example, a cDNA encoding the variable region (V region) of an anti-Notch receptor antibody is prepared from hybridoma cells expressing the anti-Notch receptor antibody. For this purpose, total RNA is first extracted from hybridomas. Methods used for extracting mRNAs from cells include, for example:
the guanidine ultracentrifugation method (Biochemistry (1979) 18(24), 5294-5299), and
the AGPC method (Anal. Biochem. (1987) 162(1), 156-159)

Extracted mRNAs can be purified using the mRNA Purification Kit (GE Healthcare Bioscience) or such. Alternatively, kits for extracting total mRNA directly from cells, such as the QuickPrep mRNA Purification Kit (GE Healthcare Bioscience), are also commercially available. mRNAs can be prepared from hybridomas using such kits. cDNAs encoding the antibody V region can be synthesized from the prepared mRNAs using a reverse transcriptase. cDNAs can be synthesized using the AMV Reverse Transcriptase First-strand cDNA Synthesis Kit (Seikagaku Co.) or such. Furthermore, the SMART RACE cDNA amplification kit (Clontech) and the PCR-based 5′-RACE method (Proc. Natl. Acad. Sci. USA (1988) 85(23), 8998-9002; Nucleic Acids Res. (1989) 17(8), 2919-2932) can be appropriately used to synthesize and amplify cDNAs. In such a cDNA synthesis process, appropriate restriction enzyme sites described below may be introduced into both ends of a cDNA.

The cDNA fragment of interest is purified from the resulting PCR product, and then this is ligated to a vector DNA. A recombinant vector is thus constructed, and introduced into E. coli or such. After colony selection, the desired recombinant vector can be prepared from the colony-forming E. coli. Then, whether the recombinant vector has the cDNA nucleotide sequence of interest is tested by a known method such as the dideoxy nucleotide chain termination method.

The 5′-RACE method which uses primers to amplify the variable region gene is conveniently used for isolating the gene encoding the variable region. First, a 5′-RACE cDNA library is constructed by cDNA synthesis using RNAs extracted from hybridoma cells as a template. A commercially available kit such as the SMART RACE cDNA amplification kit is appropriately used to synthesize the 5′-RACE cDNA library.

The antibody gene is amplified by PCR using the prepared 5′-RACE cDNA library as a template. Primers for amplifying the mouse antibody gene can be designed based on known antibody gene sequences. The nucleotide sequences of the primers vary depending on the immunoglobulin subclass. Therefore, it is preferable that the subclass is determined in advance using a commercially available kit such as the Iso Strip mouse monoclonal antibody isotyping kit (Roche Diagnostics).

Specifically, for example, primers that allow amplification of genes encoding gamma1, gamma2a, gamma2b, and gamma3 heavy chains and kappa and lambda light chains are used to isolate mouse IgG-encoding genes. In general, a primer that anneals to a constant region site close to the variable region is used as a 3′-side primer to amplify an IgG variable region gene. Meanwhile, a primer attached to a 5′ RACE cDNA library construction kit is used as a 5′-side primer.

PCR products thus amplified are used to reshape immunoglobulins composed of a combination of heavy and light chains. A desired antibody can be selected using the Notch receptor-binding activity of a reshaped immunoglobulin as an indicator. For example, when the objective is to isolate an antibody against Notch receptor, it is more preferred that the binding of the antibody to Notch receptor is specific. A Notch receptor-binding antibody can be screened, for example, by the following steps:
(1) contacting a Notch receptor-expressing cell with an antibody comprising the V region encoded by a cDNA isolated from a hybridoma;
(2) detecting the binding of the antibody to the Notch receptor-expressing cell; and
(3) selecting an antibody that binds to the Notch receptor-expressing cell.

Methods for detecting the binding of an antibody to Notch receptor-expressing cells are known. Specifically, the binding of an antibody to Notch receptor-expressing cells can be detected by the above-described techniques such as FACS. Immobilized samples of Notch receptor-expressing cells are appropriately used to assess the binding activity of an antibody.

Preferred antibody screening methods that use the binding activity as an indicator also include panning methods using phage vectors. Screening methods using phage vectors are advantageous when the antibody genes are isolated from heavy-chain and light-chain subclass libraries from a polyclonal antibody-expressing cell population. Genes encoding the heavy-chain and light-chain variable regions can be linked by an appropriate linker sequence to form a single-chain Fv (scFv). Phages presenting scFv on their surface can be produced by inserting a gene encoding scFv into a phage vector. The phages are contacted with an antigen of interest. Then, a DNA encoding scFv having the binding activity of interest can be isolated by collecting phages bound to the antigen. This process can be repeated as necessary to enrich scFv having a desired binding activity.

After isolation of the cDNA encoding the V region of the anti-Notch receptor antibody of interest, the cDNA is digested with restriction enzymes that recognize the restriction sites introduced into both ends of the cDNA. Preferred restriction enzymes recognize and cleave a nucleotide sequence that occurs in the nucleotide sequence of the antibody gene at a low frequency. Furthermore, a restriction site for an enzyme that produces a sticky end is preferably introduced into a vector to insert a single-copy digested fragment in the correct orientation. The cDNA encoding the V region of the anti-Notch receptor antibody is digested as described above, and this is inserted into an appropriate expression vector to construct an antibody expression vector. In this case, if a gene encoding the antibody constant region (C region) and a gene encoding the above V region are fused in-frame, a chimeric antibody is obtained. Herein, “chimeric antibody” means that the origin of the constant region is different from that of the variable region. Thus, in addition to mouse/human heterochimeric antibodies, human/human allochimeric antibodies are included in the chimeric antibodies of the present invention. A chimeric antibody expression vector can be constructed by inserting the above V region gene into an expression vector that already has the constant region. Specifically, for example, a recognition sequence for a restriction enzyme that excises the above V region gene can be appropriately placed on the 5′ side of an expression vector carrying a DNA encoding a desired antibody constant region (C region). A chimeric antibody expression vector is constructed by fusing in frame the two genes digested with the same combination of restriction enzymes.

To produce an anti-Notch receptor monoclonal antibody, antibody genes are inserted into an expression vector so that the genes are expressed under the control of an expression regulatory region. The expression regulatory region for antibody expression includes, for example, enhancers and promoters. Furthermore, an appropriate signal sequence may be attached to the amino terminus so that the expressed antibody is secreted to the outside of cells. In the Examples described below, a peptide having the amino acid sequence MGWSCIILFLVATATGVHS (SEQ ID NO: 17) is used as a signal sequence. Meanwhile, other appropriate signal sequences may be attached. The expressed polypeptide is cleaved at the carboxyl terminus of the above sequence, and the resulting polypeptide is secreted to the outside of cells as a mature polypeptide. Then, appropriate host cells are transformed with the expression vector, and recombinant cells expressing the anti-Notch receptor antibody-encoding DNA are obtained.

DNAs encoding the antibody heavy chain (H chain) and light chain (L chain) are separately inserted into different expression vectors to express the antibody gene. An antibody molecule having the H and L chains can be expressed by co-transfecting the same host cell with vectors into which the H-chain and L-chain genes are respectively inserted. Alternatively, host cells can be transformed with a single expression vector into which DNAs encoding the H and L chains are inserted (see WO 94/11523).

There are various known host cell/expression vector combinations for antibody preparation by introducing isolated antibody genes into appropriate hosts. All of these expression systems are applicable to isolation of domains including antibody variable regions of the present invention. Appropriate eukaryotic cells used as host cells include animal cells, plant cells, and fungal cells. Specifically, the animal cells include, for example, the following cells.
(1) mammalian cells: CHO, COS, myeloma, baby hamster kidney (BHK), HeLa, Vero, or such;
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells: yeasts: the Saccharomyces genus such as Saccharomyces cerevisiae, and the Pichia genus such as Pichia pastoris; and filamentous fungi: the Aspergillus genus such as Aspergillus niger.

Furthermore, antibody gene expression systems that utilize prokaryotic cells are also known. For example, when using bacterial cells, E. coli cells, Bacillus subtilis cells, and such can suitably be utilized in the present invention. Expression vectors carrying the antibody genes of interest are introduced into these cells by transfection. The transfected cells are cultured in vitro, and the desired antibody can be prepared from the culture of transformed cells.

In addition to the above-described host cells, transgenic animals can also be used to produce a recombinant antibody. That is, the antibody can be obtained from an animal into which the gene encoding the antibody of interest is introduced. For example, the antibody gene can be constructed as a fusion gene by inserting in frame into a gene that encodes a protein produced specifically in milk. Goat beta-casein or such can be used, for example, as the protein secreted in milk. DNA fragments containing the fused gene inserted with the antibody gene is injected into a goat embryo, and then this embryo is introduced into a female goat. Desired antibodies can be obtained as a protein fused with the milk protein from milk produced by the transgenic goat born from the embryo-recipient goat (or progeny thereof). In addition, to increase the volume of milk containing the desired antibody produced by the transgenic goat, hormones can be administered to the transgenic goat as necessary (Ebert, K. M. et al., Bio/Technology (1994) 12 (7), 699-702).

### Methods for Producing a Humanized Antibody

When an antigen-binding molecule described herein is administered to human, a domain derived from a genetically recombinant antibody that has been artificially modified to reduce the heterologous antigenicity against human and such, can be appropriately used as the domain of the antigen-binding molecule including an antibody variable region. Such genetically recombinant antibodies include, for example, humanized antibodies. These modified antibodies are appropriately produced by known methods. Furthermore, generally, the binding specificity of a certain antibody can be introduced into another antibody by CDR grafting.

Specifically, humanized antibodies prepared by grafting the CDR of a non-human animal antibody such as a mouse antibody to a human antibody and such are known. Common genetic engineering techniques for obtaining humanized antibodies are also known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the FR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, complementary strand synthesis reaction is conducted to anneal the overlapping CDR regions of the products synthesized using a human antibody gene as template. Human FRs are ligated via the mouse CDR sequences by this reaction.

The full length V region gene, in which three CDRs and four FRs are ultimately ligated, is amplified using primers that anneal to its 5′- or 3-end, which are added with suitable restriction enzyme recognition sequences. An expression vector for humanized antibody can be produced by inserting the DNA obtained as described above and a DNA that encodes a human antibody C region into an expression vector so that they will ligate in frame. After the recombinant vector is transfected into a host to establish recombinant cells, the recombinant cells are cultured, and the DNA encoding the humanized antibody is expressed to produce the humanized antibody in the cell culture (see, European Patent Publication No. EP 239400 and International Patent Publication No. WO 1996/002576).

By qualitatively or quantitatively measuring and evaluating the antigen-binding activity of the humanized antibody produced as described above, one can suitably select human antibody FRs that allow CDRs to form a favorable antigen-binding site when ligated through the CDRs. Amino acid residues in FRs may be substituted as necessary, so that the CDRs of a reshaped human antibody form an appropriate antigen-binding site. For example, amino acid sequence mutations can be introduced into FRs by applying the PCR method used for grafting a mouse CDR into a human FR. More specifically, partial nucleotide sequence mutations can be introduced into primers that anneal to the FR. Nucleotide sequence mutations are introduced into the FRs synthesized by using such primers. Mutant FR sequences having the desired characteristics can be selected by measuring and evaluating the activity of the amino acid-substituted mutant antibody to bind to the antigen by the above-mentioned method (Sato, K. et al., Cancer Res. (1993) 53: 851-856)

### Methods for Producing a Human Antibody

Alternatively, desired human antibodies can be obtained by immunizing transgenic animals having the entire repertoire of human antibody genes (see WO 1993/012227; WO 1992/003918; WO 1994/002602; WO 1994/025585; WO 1996/034096; WO 1996/033735) by DNA immunization.

Furthermore, techniques for preparing human antibodies by panning using human antibody libraries are also known. For example, the V region of a human antibody is expressed as a single-chain antibody (scFv) on phage surface by the phage display method. Phages expressing a scFv that binds to the antigen can be selected. The DNA sequence encoding the human antibody V region that binds to the antigen can be determined by analyzing the genes of selected phages. The DNA sequence of the scFv that binds to the antigen is determined. An expression vector is prepared by fusing the V region sequence in frame with the C region sequence of a desired human antibody, and inserting this into an appropriate expression vector. The expression vector is introduced into cells appropriate for expression such as those described above. The human antibody can be produced by expressing the human antibody-encoding gene in the cells. These methods are already known (see WO 1992/001047; WO 1992/020791; WO 1993/006213; WO 1993/011236; WO 1993/019172; WO 1995/001438; WO 1995/015388).

### Epitope

“Epitope” means an antigenic determinant in an antigen, and refers to an antigen site to which the antigen-binding domain of an antigen-binding molecule or antibody disclosed herein binds. Thus, for example, the epitope can be defined according to its structure. Alternatively, the epitope may be defined according to the antigen-binding activity of an antigen-binding molecule or antibody that recognizes the epitope. When the antigen is a peptide or polypeptide, the epitope can be specified by the amino acid residues forming the epitope. Alternatively, when the epitope is a sugar chain, the epitope can be specified by its specific sugar chain structure.

A linear epitope is an epitope that contains an epitope whose primary amino acid sequence is recognized. Such a linear epitope typically contains at least three and most commonly at least five, for example, about 8 to 10 or 6 to 20 amino acids in its specific sequence.

In contrast to the linear epitope, “conformational epitope” is an epitope in which the primary amino acid sequence containing the epitope is not the only determinant of the recognized epitope (for example, the primary amino acid sequence of a conformational epitope is not necessarily recognized by an epitope-defining antibody). Conformational epitopes may contain a greater number of amino acids compared to linear epitopes. A conformational epitope-recognizing antigen-binding domain recognizes the three-dimensional structure of a peptide or protein. For example, when a protein molecule folds and forms a three-dimensional structure, amino acids and/or polypeptide main chains that form a conformational epitope become aligned, and the epitope is made recognizable by the antigen-binding domain. Methods for determining epitope conformations include, for example, X ray crystallography, two-dimensional nuclear magnetic resonance, site-specific spin labeling, and electron paramagnetic resonance, but are not limited thereto. See, for example, Epitope Mapping Protocols in Methods in Molecular Biology (1996), Vol. 66, Morris (ed.).

Examples of a method for assessing the epitope binding by a test antigen-binding molecule or antibody containing an anti-Notch receptor antigen-binding domain are described below. According to the examples below, methods for assessing the epitope binding by a test antigen-binding molecule or antibody containing an antigen-binding domain for an antigen other than Notch receptor, can also be appropriately conducted.

For example, whether a test antigen-binding molecule or antibody containing an anti-Notch receptor antigen-binding domain recognizes a linear epitope in the Notch receptor molecule can be confirmed for example as mentioned below. A linear peptide comprising an amino acid sequence forming the extracellular domain of Notch receptor is synthesized for the above purpose. The peptide can be synthesized chemically, or obtained by genetic engineering techniques using a region encoding the amino acid sequence corresponding to the extracellular domain in a Notch receptor cDNA. Then, a test antigen-binding molecule or antibody containing an anti-Notch receptor antigen-binding domain is assessed for its binding activity towards a linear peptide comprising the amino acid sequence forming the extracellular domain. For example, an immobilized linear peptide can be used as an antigen by ELISA to evaluate the binding activity of the polypeptide complex towards the peptide. Alternatively, the binding activity towards a linear peptide can be assessed based on the level that the linear peptide inhibits the binding of the antigen-binding molecule or antibody to Notch receptor-expressing cells. These tests can demonstrate the binding activity of the antigen-binding molecule or antibody towards the linear peptide.

Whether a test antigen-binding molecule or antibody containing an anti-Notch receptor antigen-binding domain recognizes a conformational epitope can be assessed as follows. Notch receptor-expressing cells are prepared for the above purpose. A test antigen-binding molecule or antibody containing an anti-Notch receptor antigen-binding domain can be determined to recognize a conformational epitope when it strongly binds to Notch receptor-expressing cells upon contact, but does not substantially bind to an immobilized linear peptide comprising an amino acid sequence forming the extracellular domain of Notch receptor. Herein, “not substantially bind” means that the binding activity is 80% or less, generally 50% or less, preferably 30% or less, and particularly preferably 15% or less compared to the binding activity towards cells expressing a Notch receptor.

Methods for assaying the binding activity of a test antigen-binding molecule or antibody containing an anti-Notch receptor antigen-binding domain towards Notch receptor-expressing cells include, for example, the methods described in Antibodies: A Laboratory Manual (Ed Harlow, David Lane, Cold Spring Harbor Laboratory (1988) 359-420). Specifically, the assessment can be performed based on the principle of ELISA or fluorescence activated cell sorting (FACS) using Notch receptor-expressing cells as antigen.

In the ELISA format, the binding activity of a test antigen-binding molecule or antibody containing an anti-Notch receptor antigen-binding domain towards Notch receptor-expressing cells can be assessed quantitatively by comparing the levels of signal generated by enzymatic reaction. Specifically, a test polypeptide complex is added to an ELISA plate onto which Notch receptor-expressing cells are immobilized. Then, the test antigen-binding molecule or antibody bound to the cells is detected using an enzyme-labeled antibody that recognizes the test antigen-binding molecule or antibody. Alternatively, when FACS is used, a dilution series of a test antigen-binding molecule or antibody is prepared, and the antibody binding titer for Notch receptor-expressing cells can be determined to compare the binding activity of the test antigen-binding molecule or antibody towards Notch receptor-expressing cells.

The binding of a test antigen-binding molecule or antibody towards an antigen expressed on the cells or surface of cells suspended in buffer or the like can be detected using a flow cytometer. Known flow cytometers include, for example, the following devices:
FACSCanto™ II
FACSAria™
FACSArray™
FACSVantage™ SE
FACSCalibur™ (all are trade names of BD Biosciences)
EPICS ALTRA HyPerSort
Cytomics FC 500
EPICS XL-MCL ADC EPICS XL ADC
Cell Lab Quanta/Cell Lab Quanta SC (all are trade names of Beckman Coulter)

Preferable methods for assaying the binding activity of a test antigen-binding molecule or antibody containing an anti-Notch receptor antigen-binding domain towards an antigen include, for example, the following method. First, Notch receptor-expressing cells are reacted with a test antigen-binding molecule or antibody, and then this is stained with an FITC-labeled secondary antibody that recognizes the antigen-binding molecule or antibody. The test antigen-binding molecule or antibody is appropriately diluted with a suitable buffer to prepare the antigen-binding molecule or antibody at a desired concentration. For example, the antigen-binding molecule or antibody can be used at a concentration within the range of 10 micro g/ml to 10 ng/ml. Then, the fluorescence intensity and cell count are determined using FACSCalibur (BD). The fluorescence intensity obtained by analysis using the CELL QUEST Software (BD), i.e., the Geometric Mean value, reflects the quantity of antibody bound to cells. That is, the binding activity of a test antigen-binding molecule or antibody, which is represented by the quantity of the test antigen-binding molecule or antibody bound, can be determined by measuring the Geometric Mean (Geo-mean) value.

Whether a test antigen-binding molecule or antibody containing an anti-Notch receptor antigen-binding domain shares a common epitope with another antigen-binding molecule or antibody can be assessed based on the competition between the two antigen-binding molecules or antibodies for the same epitope. The competition between the antigen-binding molecules or antibodies can be detected by cross-blocking assay or the like. For example, the competitive ELISA assay is a preferred cross-blocking assay.

Specifically, in cross-blocking assay, the Notch receptor protein immobilized to the wells of a microtiter plate is pre-incubated in the presence or absence of a candidate competitor antigen-binding molecule or antibody, and then a test antigen-binding molecule or antibody is added thereto. The quantity of test antigen-binding molecule or antibody bound to the Notch receptor protein in the wells is indirectly correlated with the binding ability of a candidate competitor antigen-binding molecule or antibody that competes for the binding to the same epitope. That is, the greater the affinity of the competitor antigen-binding molecule or antibody for the same epitope, the lower the binding activity of the test antigen-binding molecule or antibody towards the Notch receptor protein-coated wells.

The quantity of the test antigen-binding molecule or antibody bound to the wells via the Notch receptor protein can be readily determined by labeling the antigen-binding molecule or antibody in advance. For example, a biotin-labeled antigen-binding molecule or antibody is measured using an avidin/peroxidase conjugate and appropriate substrate. In particular, cross-blocking assay that uses enzyme labels such as peroxidase is called “competitive ELISA assay”. The antigen-binding molecule or antibody can also be labeled with other labeling substances that enable detection or measurement. Specifically, radiolabels, fluorescent labels, and such are known.

When the candidate competitor antigen-binding molecule or antibody can block the binding by a test antigen-binding molecule or antibody containing an anti-Notch receptor antigen-binding domain by at least 20%, preferably at least 20 to 50%, and more preferably at least 50% compared to the binding activity in a control experiment conducted in the absence of the competitor antigen-binding molecule or antibody, the test antigen-binding molecule or antibody is determined to substantially bind to the same epitope bound by the competitor antigen-binding molecule or antibody, or compete for the binding to the same epitope.

When the structure of an epitope bound by a test antigen-binding molecule or antibody containing an anti-Notch receptor antigen-binding domain has already been identified, whether the test and control antigen-binding molecules or antibodies share a common epitope can be assessed by comparing the binding activities of the two antigen-binding molecules or antibodies towards a peptide prepared by introducing amino acid mutations into the peptide forming the epitope.

To measure the above binding activities, for example, the binding activities of test and control antigen-binding molecules or antibodies towards a linear peptide into which a mutation is introduced are compared in the above ELISA format. Besides the ELISA methods, the binding activity towards the mutant peptide bound to a column can be determined by flowing test and control antigen-binding molecules or antibodies in the column, and then quantifying the antigen-binding molecule or antibody eluted in the elution solution. Methods for adsorbing a mutant peptide to a column, for example, in the form of a GST fusion peptide, are known.

Alternatively, when the identified epitope is a conformational epitope, whether test and control antigen-binding molecules or antibodies share a common epitope can be assessed by the following method. First, Notch receptor-expressing cells and cells expressing a Notch receptor with a mutation introduced into the epitope are prepared. The test and control antigen-binding molecules or antibodies are added to a cell suspension prepared by suspending these cells in an appropriate buffer such as PBS. Then, the cell suspensions are appropriately washed with a buffer, and an FITC-labeled antibody that recognizes the test and control antigen-binding molecules or antibodies is added thereto. The fluorescence intensity and number of cells stained with the labeled antibody are determined using FACSCalibur (BD). The test and control antigen-binding molecules or antibodies are appropriately diluted using a suitable buffer, and used at desired concentrations. For example, they may be used at a concentration within the range of 10 micro g/ml to 10 ng/ml. The fluorescence intensity determined by analysis using the CELL QUEST Software (BD), i.e., the Geometric Mean value, reflects the quantity of labeled antibody bound to cells. That is, the binding activities of the test and control antigen-binding molecules or antibodies, which are represented by the quantity of labeled antibody bound, can be determined by measuring the Geometric Mean value.

In the above method, whether an antigen-binding molecule or antibody does “not substantially bind to cells expressing mutant Notch receptor” can be assessed, for example, by the following method. First, the test and control antigen-binding molecules or antibodies bound to cells expressing mutant Notch receptor are stained with a labeled antibody. Then, the fluorescence intensity of the cells is determined. When FACSCalibur is used for fluorescence detection by flow cytometry, the determined fluorescence intensity can be analyzed using the CELL QUEST Software. From the Geometric Mean values in the presence and absence of the antigen-binding molecule or antibody, the comparison value (delta Geo-Mean) can be calculated according to the following formula to determine the ratio of increase in fluorescence intensity as a result of the binding by the antigen-binding molecule or antibody. delta Geo-Mean=Geo-Mean (in the presence of the antigen-binding molecule or antibody)/Geo-Mean (in the absence of the antigen-binding molecule or antibody)

The Geometric Mean comparison value (delta Geo-Mean value for the mutant Notch receptor molecule) determined by the above analysis, which reflects the quantity of a test antigen-binding molecule or antibody bound to cells expressing mutant Notch receptor, is compared to the delta Geo-Mean comparison value that reflects the quantity of the test antigen-binding molecule or antibody bound to Notch receptor-expressing cells. In this case, the concentrations of the test antigen-binding molecule or antibody used to determine the delta Geo-Mean comparison values for Notch receptor-expressing cells and cells expressing mutant Notch receptor are particularly preferably adjusted to be equal or substantially equal. An antigen-binding molecule or antibody that has been confirmed to recognize an epitope in Notch receptor is used as a control antigen-binding molecule or antibody.

If the delta Geo-Mean comparison value of a test antigen-binding molecule or antibody for cells expressing mutant Notch receptor is smaller than the delta Geo-Mean comparison value of the test antigen-binding molecule or antibody for Notch receptor-expressing cells by at least 80%, preferably 50%, more preferably 30%, and particularly preferably 15%, then the test antigen-binding molecule or antibody “does not substantially bind to cells expressing mutant Notch receptor”. The formula for determining the Geo-Mean (Geometric Mean) value is described in the CELL QUEST Software User's Guide (BD biosciences). When the comparison shows that the comparison values are substantially equivalent, the epitope for the test and control antigen-binding molecules or antibodies can be determined to be the same.

### Production and Purification of Multispecific Antigen-Binding Molecules

In some embodiments, the multispecific antigen-binding molecules of the present disclosure are isolated multispecific antigen-binding molecules.

In one embodiment, the multispecific antigen-binding molecules described herein comprise two different antigen-binding moieties (e.g. the “first antigen-binding moiety” and the “second antigen-binding moiety”), fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of multispecific antigen-binding molecules in recombinant production, it will thus be advantageous to introduce in the Fc domain of the multispecific antigen-binding molecule a modification promoting the association of the desired polypeptides.

Accordingly, in particular embodiments, the Fc domain of the multispecific antigen-binding molecule described herein comprises a modification promoting the association of the first and the second subunit of the Fc domain. The site of most extensive protein-protein interaction between the two subunits of a human IgG Fc domain is in the CH3 domain of the Fc domain. Thus, in one embodiment, said modification is in the CH3 domain of the Fc domain.

In a specific embodiment, said modification is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the multispecific antigen-binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In other embodiments, other techniques for promoting the association among H chains and between L and H chains having the desired combinations can be applied to the multispecific antigen-binding molecules of the present disclosure.

For example, techniques for suppressing undesired H-chain association by introducing electrostatic repulsion at the interface of the second constant region or the third constant region of the antibody H chain (CH2 or CH3) can be applied to multispecific antibody association (WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:
(a) glutamic acid (E) and aspartic acid (D); and
(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present disclosure, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

In addition, other known techniques can also be used for formation of multispecific antigen-binding molecules of the present disclosure. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form multispecific antigen-binding molecules of interest.

In addition, technologies for antibody production using association of antibody CH1 and CL and association of VH and VL as described in WO 2011/028952, WO2014/018572, and Nat Biotechnol. 2014 February; 32(2):191-8; technologies for producing bispecific antibodies using separately prepared monoclonal antibodies in combination (Fab Arm Exchange) as described in WO2008/119353 and WO2011/131746; technologies for regulating association between antibody heavy-chain CH3s as described in WO2012/058768 and WO2013/063702; technologies for producing multispecific antibodies composed of two types of light chains and one type of heavy chain as described in WO2012/023053; technologies for producing multispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of multispecific antigen-binding molecules.

Alternatively, even when a multispecific antigen-binding molecule of interest cannot be formed efficiently, a multispecific antigen-binding molecule of the present disclosure can be obtained by separating and purifying the multispecific antigen-binding molecule of interest from the produced molecules. For example, a method for enabling purification of two types of homomeric forms and the heteromeric antibody of interest by ion-exchange chromatography by imparting a difference in isoelectric points by introducing amino acid substitutions into the variable regions of the two types of H chains has been reported (WO2007114325). To date, as a method for purifying heteromeric antibodies, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antibody can be purified efficiently on its own by using H chains comprising substitution of amino acid residues at EU numbering positions 435 and 436, which is the IgG-Protein A binding site, with Tyr, His, or such which are amino acids that yield a different Protein A affinity, or using H chains with a different protein A affinity, to change the interaction of each of the H chains with Protein A, and then using a Protein A column.

Furthermore, an Fc region whose Fc region C-terminal heterogeneity has been improved can be appropriately used as an Fc region of the present disclosure. More specifically, the present disclosure provides Fc regions produced by deleting glycine at position 446 and lysine at position 447 as specified by EU numbering from the amino acid sequences of two polypeptides constituting an Fc region derived from IgG1, IgG2, IgG3, or IgG4.

Multispecific antigen-binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the multispecific antigen-binding molecule binds. For example, for affinity chromatography purification of multispecific antigen-binding molecules of the invention, a matrix with protein A or protein G may be used. Sequential Protein A or G affinity chromatography and size exclusion chromatography can be used to isolate a multispecific antigen-binding molecule. The purity of the multispecific antigen-binding molecule can be determined by any of a variety of well-known analytical methods including gel electrophoresis, high pressure liquid chromatography, and the like.

### Pharmaceutical Composition

In one aspect, the present disclosure provides a pharmaceutical composition comprising the multispecific antigen-binding molecule of the disclosure.

In certain embodiments, the pharmaceutical composition of the disclosure induces trans-activation to Notch signaling pathway in the target cell of interest, in another word, the pharmaceutical composition of the disclosure is a therapeutic agent for use in treating or preventing a Notch receptor-mediated disease or disorder via (trans-)activation of the Notch signaling pathway. In certain embodiments, the pharmaceutical composition of the disclosure enhances muscle regeneration and/or maintain muscle function. In certain embodiments, the pharmaceutical composition of the disclosure enhances muscle satellite cell proliferation and differentiation.

In certain embodiments, the pharmaceutical composition of the disclosure is a pharmaceutical composition used for treatment and/or prevention of Muscular Dystrophy, Tissue fibrosis, Autoimmune diseases (e.g. SLE (systemic lupus erythematosus), RA (Rheumatoid arthritis), MS (Multiple sclerosis), etc.). In certain embodiments, the pharmaceutical composition of the disclosure is cell growth-suppressing agent. In certain embodiments, the pharmaceutical composition of the disclosure is a pharmaceutical composition used for treatment and/or prevention of any cancers and malignancies that can benefit from Notch agonist (i.e. cancers with downregulation of Notch signaling). In certain embodiments, the pharmaceutical composition of the disclosure is a pharmaceutical composition used for treatment and/or prevention of gastrointestinal cancers and malignancies that can benefit from Notch agonist (i.e. cancers with downregulation of Notch signaling). In certain embodiments, the pharmaceutical composition of the disclosure is a pharmaceutical composition used for treatment and/or prevention of DMD (Duchenne muscular dystrophy). In certain embodiments, the pharmaceutical composition of the disclosure is a pharmaceutical composition used for preventing the progression of DMD (Duchenne muscular dystrophy).

In certain embodiments, the pharmaceutical composition of the disclosure is a pharmaceutical composition used for promoting lgr5+CBC's self-renewal and/or proliferation. In certain embodiments, the pharmaceutical composition of the disclosure is a pharmaceutical composition used for treatment and/or prevention of Gastrointestinal (GI) tract diseases such as Crohn's disease and Ulcerative Colitis, IBS or any diseases that results in intestinal damage. In certain embodiments, the pharmaceutical composition of the disclosure is a pharmaceutical composition used for promoting gut repair.

Pharmaceutical compositions comprising an antigen-binding molecule or antibody as described herein are prepared by mixing such antigen-binding molecule or antibody having the desired degree of purity with one or more optional pharmaceutically acceptable carriers (Remington's Pharmaceutical Sciences 16^{th} edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. Such active ingredients are suitably present in combination in amounts that are effective for the purpose intended.

If necessary, the antigen-binding molecules or antibodies of the present disclosure may be encapsulated in microcapsules (microcapsules made from hydroxymethylcellulose, gelatin, poly[methylmethacrylate], and the like), and made into components of colloidal drug delivery systems (liposomes, albumin microspheres, microemulsions, nano-particles, and nano-capsules) (for example, see “Remington's Pharmaceutical Science 16^{th} edition”, Oslo Ed. (1980)). Moreover, methods for preparing agents as sustained-release agents are known, and these can be applied to the antigen-binding molecules of the present disclosure (J. Biomed. Mater. Res. (1981) 15, 267-277; Chemtech. (1982) 12, 98-105; U.S. Pat. No. 3,773,719; European Patent Application (EP) Nos. EP58481 and EP133988; Biopolymers (1983) 22, 547-556).

If necessary, the vectors comprising nucleic acid molecule encodes the multispecific antigen-binding molecules of the present disclosure may be introduced to subjects, to express the antigen-binding molecules or antibodies of the present disclosure directly within the subject. An example of vectors that is possible to be used is adenovirus, but not limited to. It is also possible to administer the nucleic acid molecule encodes the antigen-binding molecules or antibodies of the present disclosure directly into a subject, or transfer the nucleic acid molecule encodes the antigen-binding molecules or antibodies of the present disclosure via electroporation to a subject, or administer cells comprises nucleic acid molecule encodes the antigen-binding molecules or antibodies of the present disclosure to be expressed and secreted into a subject, to express and secrete the antigen-binding molecules or antibodies of the present disclosure in the subject continuously.

The pharmaceutical compositions of the present disclosure may be administered either orally or parenterally to patients. Parental administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present disclosure can be administered locally or systemically by injection. Furthermore, appropriate administration methods can be selected according to the patient's age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present disclosure is not limited to these doses.

In the present disclosure, “contact” can be carried out, for example, by adding an antigen-binding molecule of the present disclosure to culture media of cells expressing CLDN6 cultured in vitro. In this case, an antigen-binding molecule to be added can be used in an appropriate form, such as a solution or solid prepared by lyophilization or the like. When the antigen-binding molecule of the present disclosure is added as an aqueous solution, the solution may be a pure aqueous solution containing the antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The added concentration is not particularly limited; however, the final concentration in a culture medium is preferably in a range of 1 pg/ml to 1 g/ml, more preferably 1 ng/ml to 1 mg/ml, and still more preferably 1 micro g/ml to 1 mg/ml.

In one aspect, the present disclosure provides a method for activating Notch signaling pathway in a first target cell, comprising contacting the first target cell with an effective amount of the multispecific antigen-binding molecule of any aspect/embodiment of the present disclosure. In one embodiment, the first target cell is in a mammalian subject in vivo. In a further embodiment, the subject is a human. In some embodiments, the subject is a non-human mammal. In some embodiments, the method is a method for activating Notch signaling pathway in vivo. In some embodiments, the method is a method for activating Notch signaling pathway in vitro.

In an aspect, the present disclosure provides a multispecific antigen-binding molecule of any aspect/embodiment of the present disclosure for use in activating Notch signaling pathway in a first target cell.

In an aspect, the present disclosure provides a multispecific antigen-binding molecule of any aspect/embodiment of the present disclosure for use in a method for activating Notch signaling pathway in a first target cell, where the method comprises contacting the first target cell with an effective amount of the multispecific antigen-binding molecule.

In an aspect, the present disclosure provides use of a multispecific antigen-binding molecule of any aspect/embodiment of the present disclosure in the manufacture of an agent or composition (including a therapeutic agent or pharmaceutical composition) for activating Notch signaling pathway in a first target cell.

In an aspect, the present disclosure provides use of a multispecific antigen-binding molecule of any aspect/embodiment of the present disclosure in the manufacture of an agent or composition (including a therapeutic agent or pharmaceutical composition) for use in a method for activating Notch signaling pathway in a first target cell, where the method comprises contacting the first target cell with an effective amount of the multispecific antigen-binding molecule.

In an aspect, the present disclosure provides use of a multispecific antigen-binding molecule of any aspect/embodiment of the present disclosure for activating Notch signaling pathway in a first target cell, where the method comprises contacting the first target cell with an effective amount of the multispecific antigen-binding molecule.

In another embodiment of the present disclosure, “contact” can also be carried out by administration to nonhuman animals transplanted with Notch receptor-expressing cells in vivo, to animals having cells expressing a Notch receptor endogenously or in vitro condition using Notch receptor-expressing cells. The administration method may be oral or parenteral. Parenteral administration is particularly preferred. Specifically, the parenteral administration method includes injection, nasal administration, pulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present disclosure can be administered locally or systemically by injection. Furthermore, an appropriate administration method can be selected according to the age and symptoms of an animal subject. When the antigen-binding molecule is administered as an aqueous solution, the solution may be a pure aqueous solution containing the antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The administered dose can be selected, for example, from the range of 0.0001 to 1,000 mg per kg of body weight for each administration. Alternatively, the dose can be selected, for example, from the range of 0.001 to 100,000 mg/body for each patient. However, the dose of an antigen-binding molecule of the present disclosure is not limited to these examples.

The present disclosure also provides kits for use in a method of the present disclosure, which contain an antigen-binding molecule of the present disclosure or an antigen-binding molecule produced by a method of the present disclosure. The kits may be packaged with an additional pharmaceutically acceptable carrier or medium, or instruction manual describing how to use the kits, etc.

In another aspect of the invention, an article of manufacture containing materials useful for activating the Notch signaling pathway, or the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Package Insert

The term “package insert” is used to refer to instructions customarily included in commercial packages of therapeutic products, that contain information about the indications, usage, dosage, administration, combination therapy, contraindications and/or warnings concerning the use of such therapeutic products.

### Pharmaceutical Formulation

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

### Pharmaceutically Acceptable Carrier

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding molecules or antibodies of the present disclosure are used to delay development of a disease or to slow the progression of a disease.

### Other Agents and Treatments

The multispecific antigen-binding molecules described herein may be administered in combination with one or more other agents in therapy. For instance, a multispecific antigen-binding molecules as described herein may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of multispecific antigen-binding molecules used, the type of disorder or treatment, and other factors discussed above. The multispecific antigen-binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the multispecific antigen-binding molecules described herein can occur prior to, simultaneously, and/or following, administration of the additional therapeutic agent and/or adjuvant. Multispecific antigen-binding molecules as described herein can also be used in combination with radiation therapy.

All documents cited herein are incorporated herein by reference.

The following are examples of methods and compositions of the present disclosure. It is understood that various other embodiments may be practiced, given the general description provided above.

### EXAMPLES

The following are examples of methods and compositions of the invention. It is understood that various other embodiments may be practiced, given the general description provided above.

### Example 1

The Notch agonist domain is capable of activating the Notch signalling upon concurrent binding to Notch reporter and binding to the anchor antigen by the site-specific binding domain (FIGS. 1A-C). Notch agonist domain includes any polypeptide which could bind and activate Notch signalling in an anchorage dependent manner (i.e. concurrent binding of site specific binding domain to its anchor antigen). The Notch agonist domain includes the extracellular domains of Notch ligands such as Jagged1, Jagged2, DLL1, DLL3 and DLL4, or Notch agonist antibody arm. Unlike conventional agonist antibodies or ligands which could activate receptors upon binding, an antigen-binding molecule of the present invention can lead to Notch activation only when it is binding to an anchor antigen expressed on cell or cell surface or immobilized to a scaffold.

Tissue or site specificity is conferred by site-specific binding domain's selective binding to a unique anchor antigen which expression is exclusive or limited to the tissue or cell population of interest. The concept of site specific Notch trans-activation can be achieved by adopting the various multi-specific antibody formats as illustrated in FIG. 1 and the choice of anchor antigen. Most of the Notch agonist antibodies described in Examples have adopted the antibody format as illustrated in FIG. 1A with an antibody arm with binding specificity to anchor antigen (e.g. GPC3, CACNA1S and FAP).

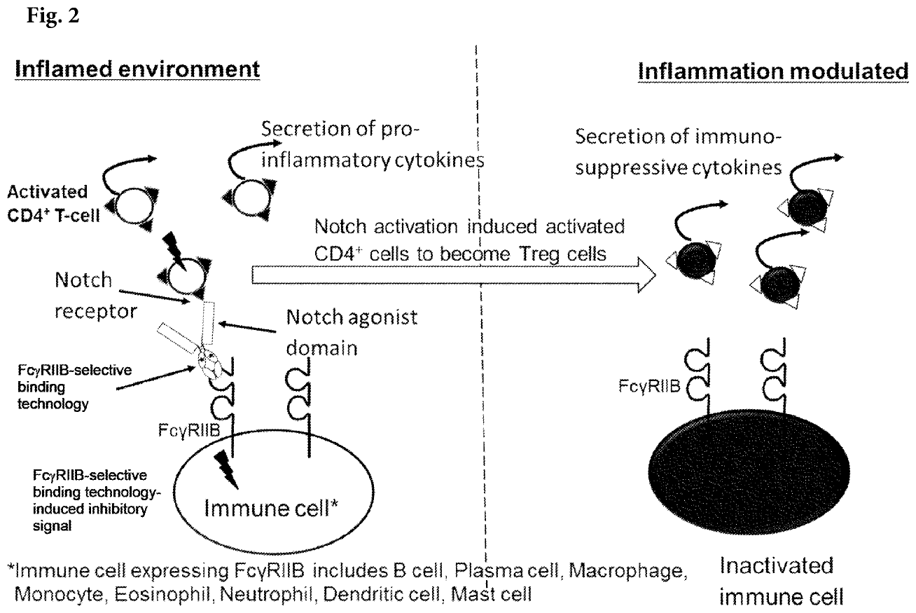

Apart from site specificity conferred by a Fab arm, an engineered Fc with enhanced binding affinity towards an anchor antigen can also provide the anchorage required for trans-activation of Notch receptors (FIG. 1B). For example, Fc gamma RIIB-selective binding technology can be applied to engineer the Fc to enhance selective binding towards Fc gamma RIIB, a membrane protein expressed by lymphoid- and myeloid-lineage cells (FIG. 2; see, e.g., WO2012/115241, WO2014/030728, WO2014/163101, WO2013/002362, WO2014/030750, WO2014/104165). Immune cells that express Fc gamma RIIB such as dendritic cells (DCs), macrophages, activated neutrophils, mast cells and basophils which are often recruited to inflammatory sites in response to chemokines. An inflammatory microenvironment is sustained through a positive feedback loop as these immune cells continue to secrete pro-inflammatory cytokines. Notch activation in activated CD4 T-lymphocytes have been reported to modulate activated CD4-T lymphocytes to become Treg cells which are capable of releasing anti-inflammatory cytokines (Brandstadter and Maillard (2019); Ferrandino et al (2018) Tindemans et al (2017)). With Fc gamma RIIB-selective binding technology applied to Notch agonist antibody, Notch activation of activated CD4 T-lymphocytes can be localized to the inflammatory sites enriched with FcgRIIB expressing cells which are in close proximity to activated CD4 T-lymphocytes in order to modulate the pro-inflammatory microenvironment (FIG. 2). Moreover, additional site specificity can be achieved by having a second antibody Fab arm which binds to a second anchor antigen (FIG. 1C). This can further enhance localized Notch trans-activation to specific cell population within the microenvironment if the second anchor antigen is exclusively expressed by the cell population of interest.

TABLE 1A
List of anchor antigen candidates which define tissue or site specificity

| | Subcellular | | |
|---|---|---|---|
| Anchor antigen | localization | Tissue/cell expression | Relevant pathology |
| Calcium Voltage-Gated | Membrane | Skeletal muscle | Muscular |
| Channel Subunit | | | dystrophic |
| Alpha1 S (CACNA1S) | | | conditions |
| Fibroblast activation | Membrane | Activated fibroblast | Tissue fibrosis |
| protein (FAP) | | | |
| FcγRIIB (CD32B) | Membrane | circulating B lymphocytes, | Autoimmune |
| | | monocytes, neutrophils, | diseases |
| | | myeloid dendritic cells | (e.g. SLE, RA and |
| | | (DCs) | MS) |

TABLE 1B
List of extracellular proteins with exclusive or limited expression in specific tissue

| List of extracellular proteins as anchor antigen candidates | Tissue |
|---|---|
| Excitatory amino acid transporter 1 | Brain |
| Glutamate [NMDA] receptor subunit zeta 1 | Brain |
| Immunoglobulin superfamily, member 8 | Brain |
| Neuronal cell adhesion molecule | Brain |
| 10 days neonate cortex cDNA, RIKEN library, clone: A830029E02 product: | Brain |
| weakly similar to BK134P22.1 | |
| N-CAM 180 of Neural cell adhesion molecule 1, 180 kDa isoform | Brain |
| Sodium/potassium-transporting ATPase beta-2 chain | Brain |
| DSD-1-proteoglycan | Brain |
| Adult male testis cDNA, synaptic vesicle glycoprotein 2 b | Brain |
| Hepatocyte cell adHesion molecule | Brain |
| Solute carrier family 12 member 5 | Brain |
| Contactin-associated protein-like 2 | Brain |
| Adult male brain UNDEFINED_CELL_LINE cDNA, Proton myo-inositol | Brain |
| transporter homolog | |
| LOC237403 protein | Brain |
| Neurofascin | Brain |
| Contactin-associated protein 1 | Brain |
| Splice Isoform 1 of Chondroitin sulfate proteoglycan 5 | Brain |
| Visual cortex cDNA, RIKEN library, clone: K530020M04 | Brain |
| product: dipeptidylpeptidase 6, full insert sequence | |
| Sodium channel beta-1 subunit precursor | Brain |
| Niemann-Pick C1-like protein 1 | Intestine |
| Oligopeptide transporter, small intestine isoform | Intestine |
| Angiotensin-converting enzyme 2 | Intestine |
| Adult male colon cDNA, RIKEN full-length enriched library, membrane-bound | Intestine |
| aminopeptidase P | |
| NOD-derived CD11c +ve dendritic cells cDNA, hypothetical protein | Intestine |
| 4 days neonate male adipose cDNA, N-acylsphingosine amidohydrolase 2 | Intestine |
| Oligopeptide transporter, small intestine isoform | Intestine |
| Calcium activated chloride channel | Intestine |
| N-acetylated-alpha-linked acidic dipeptidase-like protein | Intestine |
| Tumor necrosis factor receptor superfamily member 13C | Spleen |
| Cannabinoid receptor 2 | Spleen |
| Splice Isoform 1 of B-cell receptor CD22 | Spleen |
| Semaphorin-4D | Spleen |
| Thrombospondin 1 | Spleen |
| Osteoclast-like cell cDNA, granulin | Spleen |
| NOD-derived CD11c +ve dendritic cells cDNA, hypothetical Phospholipase | Spleen |
| D/Transphosphatidylase | |
| L-selectin | Spleen |
| Bone marrow macrophage cDNA, solute carrier family 30 | Spleen |
| B-cell differentiation antigen CD72 | Spleen |
| Transmembrane glycoprotein NMB | Spleen |
| Class II histocompatibility antigen, M beta 1 chain | Spleen |
| Splice Isoform 2 of Sialoadhesin | Spleen |
| Myeloperoxidase | Spleen |
| Leukocyte surface antigen CD53 | Spleen |
| CD180 antigen | Spleen |
| Receptor-type tyrosine-protein phosphatase eta | Spleen |
| Toll-like receptor 9 | Spleen |
| Complement receptor type 2 precursor | Spleen |
| Beta-microseminoprotein | Prostate |
| Adult male urinary bladder cDNA, hypothetical Kazal-type serine protease | Prostate |
| inhibitor domain containing protein | |
| Putative polypeptide N-acetylgalactosaminyltransferase-like protein 4 | Prostate |
| Carcinoembryonic antigen-related cell adhesion molecule 10 | Prostate |
| Adult male tongue cDNA, hypothetical protein | Prostate |
| Seminal vesicle antigen | Prostate |
| Adult male urinary bladder cDNA, weakly similar to LYSOZYME C, TYPE M | Prostate |
| Beta-defensin 50 | Prostate |
| Sodium/bile acid cotransporter | Liver |
| Asialoglycoprotein receptor major subunit | Liver |
| Similar to Rattus norvegicus putative integral membrane transport protein | Liver |
| SLC10A5 | Liver |
| Adult male testis cDNA, similar to PUTATIVE METALLOPEPTIDASE | Testis |
| Zona pellucida sperm-binding protein 3 receptor | Testis |
| Testis-specific protein TES101RP | Testis |
| Dickkopf-like protein 1 | Testis |
| Oviduct-specific glycoprotein | Ovary |
| Procollagen-lysine, 2-oxoglutarate 5-dioxygenase 2 | Ovary |
| Cathepsin L | Ovary |
| Renal sodium-dependent phosphate transport protein 2 | Kidney |
| Gamma-glutamyltranspeptidase 1 | Kidney |
| Splice Isoform 4 of Ssodium- and chloride-dependent transporter XTRP2 | Kidney |
| PREDICTED: similar to low density lipoprotein receptor-related protein 2 | Kidney |
| EP1 | Stomach |
| Potassium-transporting ATPase beta chain | Stomach |
| Secreted gel-forming mucin | Stomach |
| MUC6 | Stomach |
| Lymphocyte antigen 6 complex locus G6C protein | Epidermis |
| Sodium-dependent noradrenaline transporter | Epidermis |
| Solute carrier family 2 (facilitated glucoSe tranSporter), member 4 | Heart |
| Histidine-rich calcium-binding protein | Heart |
| Cadherin-13 | Heart |

There are several criteria that should be considered for the selection of anchor antigen. Refer to Table 1A (for the list of candidate anchor antigens), Table 1B (for the list of extracellular proteins which expression is exclusive or limited to specific tissue) or an engineered Fc with preferential binding to an anchor antigen (e.g. Fc gamma RUB-selective binding technology and Fc gamma RUB).
1) The spatial expression of the anchor antigen should be restricted to or exclusively expressed by the cell type or tissue of interest to limit systemic exposure and minimize risk of toxicity from Notch activation.
2) The temporal expression of the anchor antigen should be carefully considered. For example, some anchor antigens are only expressed in stem cells and will be lost after commitment to differentiation. Notch activation at different developmental stages also results in different phenotypes in transgenic mice. Early Notch activation leads to embryonic lethality and impaired muscle development. On the contrary, Notch activation in post-natal transgenic mice helps to improve aged muscle and boost muscle regeneration.
3) The anchor antigen should have stable expression on the cell or anchor to the cell surface with slow internalization.
4) The anchor antigen should be uniformly expressed in most cells or tissues of interest with low heterogeneity to minimize uneven activation of Notch signalling.
5) The anchor antigen should be expressed at sufficient levels even in pathological conditions and ensure sufficient retention of bi-specific Notch agonist antibody.

### Example 2. Preparation of Multi-Specific Notch Agonistic Antibody

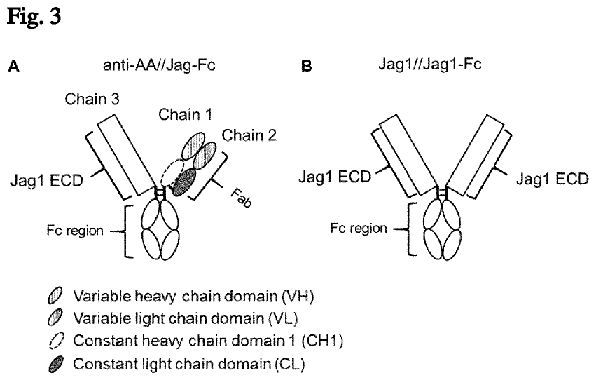

FIG. 3A shows an example of bispecific molecule, Anti-AA//Jag-Fc with one arm targeting an anchor antigen and the other arm targeting Notch receptor, consisting of an Fc, devoid of Fc gamma R binding, a human Jag1 extra cellular domain (ECD), and a Fab, binding to an anchor antigen such as GPC3. Heterodimerization and correct assembly are achieved by knob into hole (kih) mutation in the Fc. The molecule design and naming rule is showed in FIG. 3A, and the sequence ID (SEQ ID NO) is showed in Table 2A. FIG. 3B shows another example, a molecule with bivalent binding to Notch receptor Jag1//Jag1-Fc, consisting of an Fc, devoid of Fc gamma R binding, and two human Jag1 extra cellular domains (ECDs). The molecule design and naming rule is showed in FIG. 3B, and the sequence ID (SEQ ID NO) is showed in Table 2B. As shown in FIG. 3A, “Chain 1” comprises the variable heavy chain domain (VH) and constant heavy chain domain 1 (CH1) (site-specific binding domain), and Fc region; “Chain 2” comprises the variable light chain domain (VL) (site-specific binding domain) and constant light chain domain (CL); and “Chain 3” comprises a Notch agonist domain (Jag1 ECD in this Example) and the Fc region.[0301] Table 2

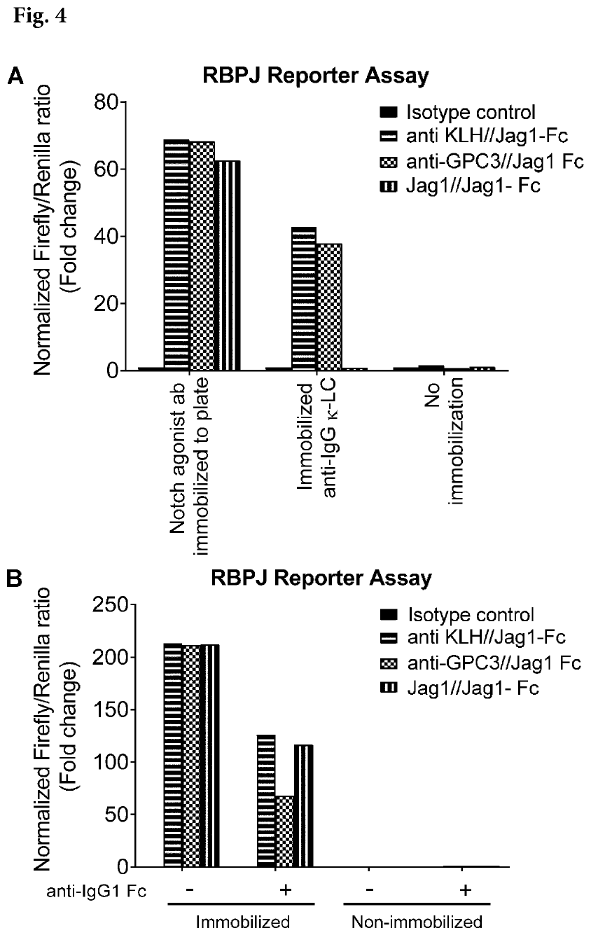

TABLE 2
Table showing sequence ID of molecules illustrated in FIG. 4
A

| | SEQ ID NO: | | |
|---|---|---|---|
| Molecule name | Chain 1 | Chain 2 | Chain 3 |
| anti KLH//Jag1-Fc | 1 | 2 | 3 |
| anti-GPC3//Jag1-Fc | 5 | 6 | 3 |

B

| Sample name | SEQ ID NO: |
|---|---|
| Jag1//Jag1-Fc | 4 |

TABLE 3
Table showing amino acid sequence of molecules illustrated FIG. 4 and Table 2

| SEQ | |
|---|---|
| ID NO | Amino Acid Sequence |
| 1 | QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLA |
| | PSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPELRRGPSVFLFPPKPKDTLMISRTPE |
| | VTCWVDVSHEDPEVKFNWYVDGVEVHNAKTKPREEQYNSTYRVVSVLTVLHQDWLNGKEYKCKVSNKGLPSSIEKTISKAKGQPREPQVYTLPPSRCEMTKNQVSLSCAVKGFYPSDIAVEWESNGQP |
| | ENNYKTTPPVLDSDGSFFLVSKLTVDKSRWQQGNVFSCSYMHEALHNHYTQKSLSLSP |
| 2 | DIQMTQSSSSFSVSUGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSG |
| | TASVVCLLNNFYPREAKVQWKVDNALQSGNSQESVTEQDSKDSTYSLSSTLTLSKADYEKHKVYACEVTHQGLSSPVTKSENRGEC |
| 3 | KVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIEKA |
| | SHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCL |
| | CETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNINCSHGGTCQDLVNGFKCVCPPQWTGKTCQ |
| | LDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGA |
| | QCYNRASDYFCKCPEDYEGKNCSHLKDHCRITPCEVIDSCTVAMASNDTPEGVRYISSNVCGPHGKCKSQSGGKFTCDCNKGFTGTYCHENINDCESNPCRNGGTCIDGVNSYKCICSDGWEGAYCET |
| | NINDCSQNPCHINGGTCRDLVNDPYCDCKNGWKGKTCHSRDSQCDEATCNINGGTCYDEGDAFKCMICPGGWEGTTCNIARNSSCLPNPCHINGGTCVVNGESFTCVCKEGWEGPICAQNTNDCSPHP |
| | CYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCI |
| | PILDDQCFVHPCTGVGECRSSSLQPVKTKCTSDSYYQDNCANITFTENKEMMSPGLTTEHICSELRNLNILKNVSAEIYSIYIACEPSPSANNEIHVAISAEDIRDDGNPIKEITDKIIDLVSKRDGN |
| | SSLIAAVAEVRVQRRPLKNRTDEPKSSDKTHTCPPCPAPELRRGPSVFLFPPKPKDTUMISRTPEVTCVVVDVSHEDPEVKFNWYVDGVEVHNAKTKPREEQYNSTYRVVSVLTVLHQDWLNGKEYKC |
| | KVSNKGLPSSIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPHHHH |
| | HHHH |
| 4 | KVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSNIEK |
| | ASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQC |
| | LCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNINCSHGGTCQDLVNGFKCVCPPQWTGKTC |
| | QLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNG |
| | AQCYNRASDYFCKCPEDYEGKINCSHLKDHCRITPCEVIDSCTVAMASNDTPEGVRYISSNVCGPHGKCKSQSGGKFTCDCNKGFTGTYCHENINDCESNPCRNGGTCIDGVNSYKCICSDGWEGAYC |
| | ETNINDCSQNPCHINGGTCRDLVNDFYCDCKINGWKGKTCHSRDSQCDEATCNNGGTCYDEGDAFKCMCPGGWEGTTCNIARNSSCLPNPCHINGGTCVVNGESFTCVCKEGWEGPICAQNTNDCSPH |
| | PCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSC |
| | IPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIDLVSKRDGNS |
| | SLIAAVAEVRVQRRPLKNRTDEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWINGKEYKCK |
| | VSNKGLPSSIEKTISKAKGQPREPQWTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP |
| 5 | QVQLVQSGAEVKKPGASVKVSCKASGYTFTDYEMHWRQPPGQGLEWIGAIDGKTPDTAYSQKFKGRVTLTADKSTSTAYMELSSLTSEDTAVYYCTRFYSYTYWGQGTLVTVSSASTKGPSVFPLAPS |
| | SKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRCEMTKNQVSISCAVKGFYPSDIAVEWESNGQP |
| | ENNYKTTPPVLDSDGSFFLVSKLTVDKSRWQQGNVFSCSVMHEALHNHYTQKSLSLSP |
| 6 | DIVMTQSPLSLPVTPGEPASISCRSSQSLVHSNRNTYLQWYQQKPGQAPRLLIYKVSNRFRGVPDRFSGSGSGTDFTLKISRVEAEDVGWYCIQNTHVPPTFGQGTKLEIKRTVAAPSVFIFPPSDEQ |
| | LKSGTASVVCLLNNFYPREAKVQWKVDNALQSGNSQESVTEQDSKDSTYSLSSTLTLSKADYEKHKVYACEVTHQGLSSPVTKSFNRGEC |

### Example 3. Purification of Multi-Specific Notch Agonistic Antibodies

Recombinant multi-specific Notch agonistic antibodies were expressed transiently using HEK293 cells. Conditioned media expressing an antibody was applied to a column packed with Protein A resin and eluted with an acidic solution. Fractions containing the antibody were collected and subsequently subjected to a gel filtration column equilibrated with Histidine buffer. Fractions containing the antibody were then pooled and stored at −80 degrees Celsius (C).

### Example 4

To prove the hypothesis that Notch agonist antibodies require anchorage to activate Notch receptor, Notch agonist antibodies were either immobilised directly to the culture plate by adsorption or captured by an anti-human kappa light chain antibody (FIG. 4A). In the absence of anchorage, the non-immobilized Notch agonist antibodies failed to activate Notch signalling in C2C12 reporter cells even after 48 hours of treatment. On the contrary, when the Notch agonist antibodies were immobilized to either the culture plate or captured by the anti-human kappa light chain antibody, significant Notch activation was observed in the C2C12 reporter cells. As Jag1//Jag1-Fc lacked the human kappa light chain, the immobilized anti-human kappa light chain antibody failed to capture Jag1//Jag1-Fc leading to its failure to activate Notch signalling in C2C12 reporter cells. This observation suggests that the activation of Notch signalling induced by the Notch agonist antibodies are dependent on anchorage.

To further elucidate the mechanism of Notch activation induced by the Notch agonist antibodies are dependent on anchorage but not clustering of antibodies by the immobilized anti-IgG antibody, an anti-human IgG Fc specific antibody was either immobilized on the culture plate via adsorption or added to culture medium along with the Notch agonist antibodies (FIG. 4B). Consistently, all Notch agonist antibodies except for the isotype control successfully induced Notch activation when they were either adsorbed to the culture plate or captured by the immobilized anti-human IgG Fc antibody. Interestingly, the addition of the anti-human Fc antibody with Notch agonist antibodies failed to activate Notch signalling. This observation suggests that the clustering or oligomerization of Notch antibodies by the anti-human IgG Fc antibody is likely to contribute to the activation of Notch signalling induced by the Notch agonist antibodies when they are anchored by the immobilized anti-human IgG Fc antibody. Similarly, it is also unlikely that Jag1//Jag1-Fc is able to crosslink Notch receptors expressed on two different cells simultaneously to induce trans-activation (FIG. 4B). The results demonstrated the importance of anchorage in order for Notch agonist antibodies to induce trans-activation of Notch signalling.

### Method for Antibody Immobilization Assay

For direct immobilization of Notch agonist antibody via absorption, a 96-well plate was first coated with the antibodies at a concentration of 10 mcg/mL for 16 hours at 4 degrees Celsius (C). For antibody capture condition, either anti-human kappa light chain antibody (in FIG. 4A; 10 mcg/mL) or anti-human IgG Fc specific antibody (in FIG. 4B; 10 mcg/mL) was first coated in the same condition as above. After antibody coating, the culture plates were washed with cell culture medium before blocking with 5% FBS solution for 2 hours at room temperature. For antibody capture condition, the Notch agonist antibodies (10 mcg/mL) were added and incubated at 37 degrees C. for 1 hour. Thereafter, C2C12 Notch reporter cells were seeded at 3×10⁴ cells per well and incubated at 37 degrees C. for 48 hours before performing dual-glo luciferase assay as per manufacturer's protocol (Promega). The luciferase signal was expressed as Firefly over renilla signal and the ratio was further normalized to the isotype control's ratio.

### Example 5

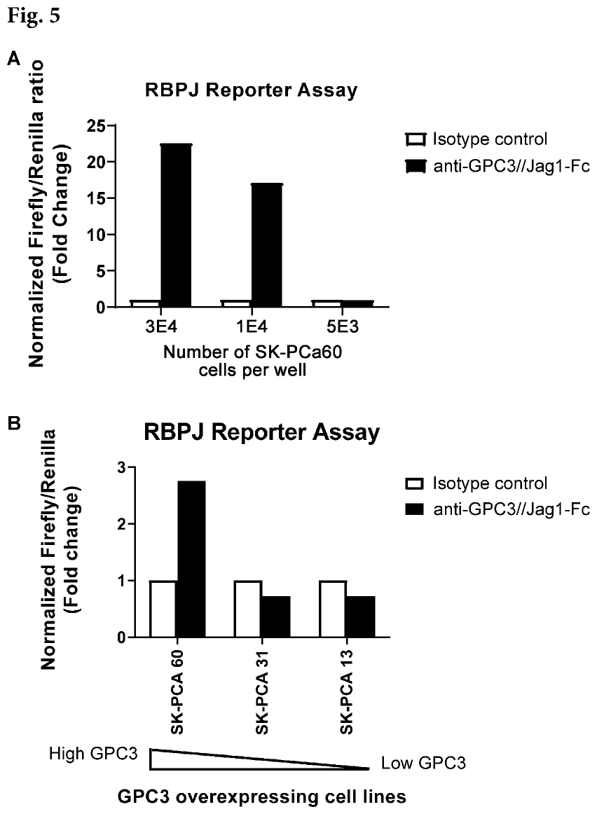

Glypican 3 (GPC3) was chosen as a model anchor antigen to demonstrate the dependency on anchorage to induce trans-activation of Notch signalling due to the availability of a well characterized anti-GPC3 antibody and a panel of transfectant cell lines with various GPC3 expression levels. The anchor antigen-expressing cell, SK-PCa60 was first co-cultured at different cell density with C2C12 Notch reporter cells treated either with anti-GPC3//Jag1-Fc or an isotype control antibody (FIG. 5A). The results show that the level of Notch activation as indicated by the luciferase activity was dependent on the number of GPC3 expressing cells. Of note, when there is insufficient anchor antigen-expressing cells (i.e. 5E3/well), anti-GPC3//Jag1 Fc failed to induce trans-activation of Notch signalling in the C2C12 reporter cells. To further validate the importance of anchor antigen expression in the anchorage dependent trans-activation of Notch signalling, SK-HEP1 cells stably transfected with various levels of GPC3 expression was co-cultured with C2C12 reporter cells treated with anti-GPC3//Jag1-Fc. Consistent with the observation in FIG. 5A, only SK-PCa60 cells (with high GPC3 expression) managed to induce Notch activation in C2C12 reporter cells after anti-GPC3//Jag1-Fc treatment (FIG. 5B).

### Method for Co-Culture Notch Reporter Assay

Anchor antigen expressing cells (i.e. GPC3 overexpressing cells) was seeded at a density of 1×10⁵ cells per well and incubated at 37 degrees C. for 16 hours. Anti-GPC3//Jag1-Fc bi-specific antibody or IgG1 isotype control antibody was added at 250 mcg/mL per well and incubated for 1 hour before 3×10⁴ of C2C12 Notch reporter cells were seeded to the wells. After 24 hours of incubation at 37 degrees C., dual-glo luciferase assay was conducted according to manufacturer's protocol (Promega). The luciferase signal was expressed as Firefly over renilla signal and the ratio was further normalized to the isotype control's ratio.

### Example 6

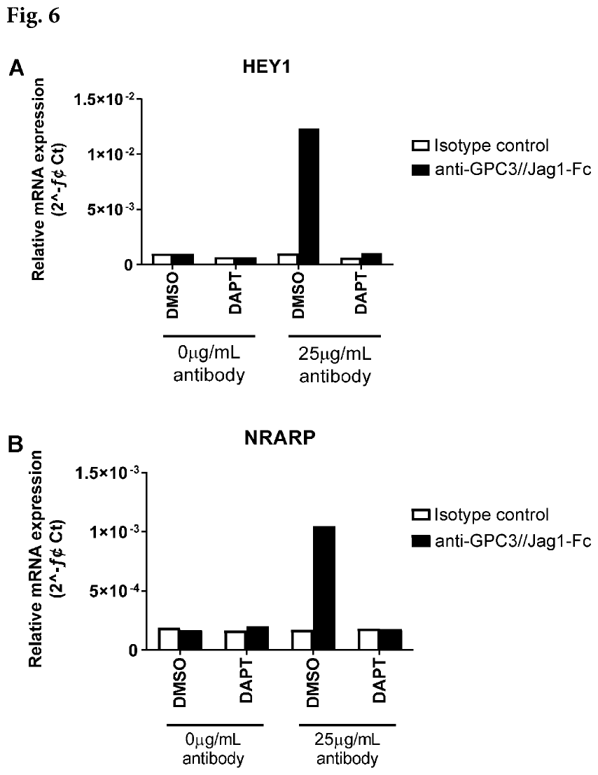

To validate the specificity of Notch signalling activation induced by anti-GPC//Jag1-Fc, the antibodies were first immobilized to a culture plate via adsorption before parental C2C12 cells were seeded (FIG. 6). The cells were treated with either DMSO as control or DAPT, a gamma-secretase inhibitor (10 micromolar) which in turn inhibits the Notch signalling pathway. Consistently, C2C12 cells with immobilized anti-GPC3//Jag1-Fc showed strong upregulation of Notch target genes, HEY1 and NRARP after 24 hours of treatment. Consequently, DAPT-treated C2C12 cells completely abrogated the Notch activation induced by anti-GPC3//Jag1-Fc, indicating that the activation was specific to Notch signalling.

### Example 7

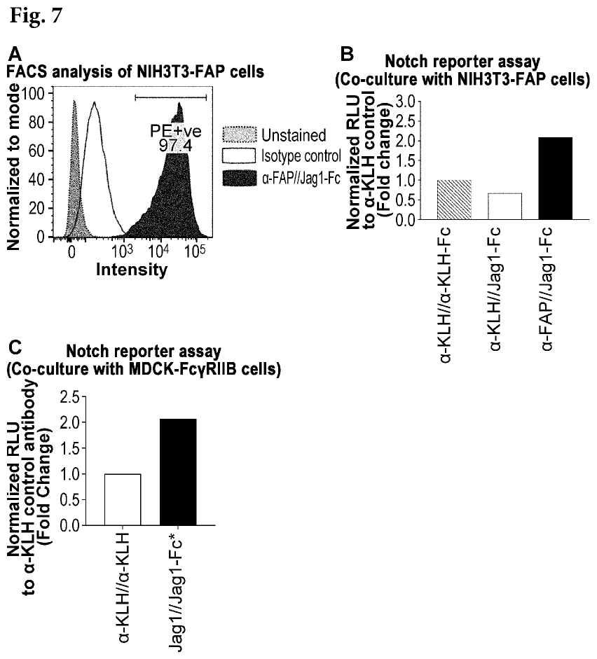

In Example 4, bispecific antibody with Jag1 ECD and anti-GPC3 binding arm demonstrated anchorage dependent trans-activation of Notch signaling. To demonstrate that the anchorage dependent trans-activation of Notch signaling is applicable to other anchor antigens, we have prepared bispecific antibodies that bind Jag1 ECD and other anchor antigens such as Fibroblast associated protein (FAP) and Fc gamma receptor IIB (Fc gammaRIIB). A bispecific antibody with Jag1 ECD and anti-FAP antibody arm (“anti-FAP//Jag1-Fc”) was generated and was shown to bind to NIH-3T3 cells overexpressing FAP (FIG. 7A). Consistent to our observation in Example 4 that Notch signaling is anchorage dependent, when NIH3T3-FAP cells were co-cultured with the Notch reporter cells, anti-FAP//Jag1-Fc was able to induce Notch activation in C2C12 Notch reporter cells but not KLH//Jag1-Fc nor KLH//KLH-Fc which were unable to bind on NIH3T3-FAP cells (FIG. 7B).

In addition, bispecific antibody (“Jag1//Jag1-Fc”) consisting of Jag1 ECD on both arms and an engineering Fc that preferentially binds to Fc gamma Receptor IIB as shown in FIG. 3B was also prepared. Using a stable cell line which overexpressed Fc gamma Receptor IIB, Jag1//Jag1-Fc* treatment was able to induce Notch activation of the Notch receptor cells (FIG. 7C). The data showed that anchorage provided by Fc instead of an antibody Fab arm, was also able to induce Notch activation. Taken together, our data suggested that the concept of anchorage dependent Notch activation induced by bispecific antibody could be applied to across anchor antigens and formats

### Example 8

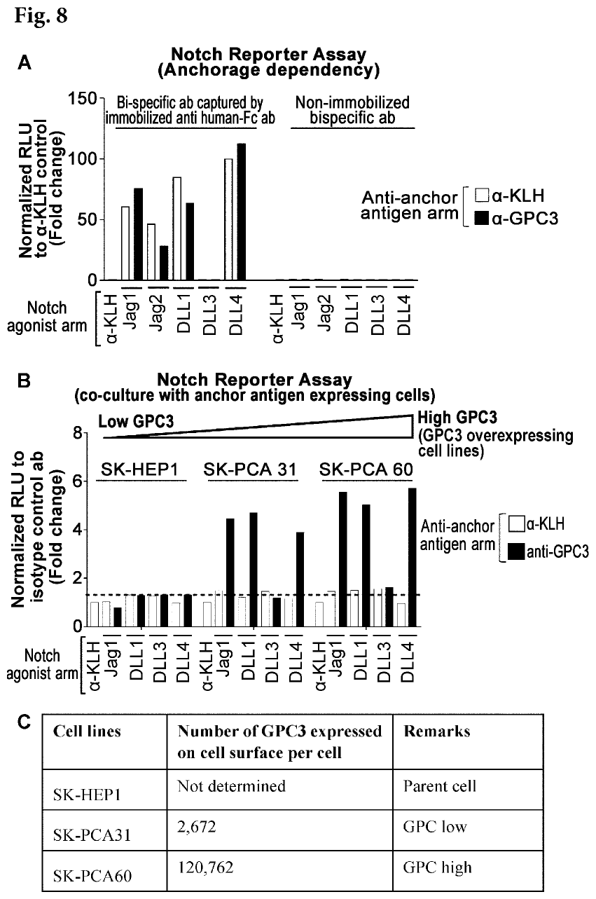

The mammalian Notch receptor family consist of four heterodimeric paralogs (Notch1-4) and they interact with five Notch ligands in the Jagged (Jag1 and Jag2) and Delta-like (DLL1, DLL3 and DLL4) families. Most Notch ligands activate Notch signaling, except DLL3 which is thought to function as a natural antagonist of signaling pathway (Kopan and Ilagan, 2009). Bi-specific antibodies consisting of a Notch ligand ECD were either immobilized to culture plate or added directly to culture medium (i.e. non-immobilized) before Notch reporter cells were added. Consistently, anchorage dependent activation of Notch reporters was observed for all Notch ligand bi-specific antibodies, except DLL3, only when they were immobilized (FIG. 8A). To demonstrate the importance of anchorage for antibody induced Notch activation, SK-HEP1 cells expressing two different levels of anchor antigen, GPC3 were used (FIG. 8B). Bi-specific antibodies consisting of anti-GPC3 binding arm were able to activate Notch reporter cells but not the anti-KLH containing bi-specific antibodies which do not bind to GPC3 expressing cells. In addition, Notch activation induced by anti-GPC3 bi-specific antibodies was only observed in GPC3 over-expressing cells (SK-PCA31 and SK-PCA60). Of note, the level of Notch activation between the SK-PCA31 (GPC3 low) and SK-PCA60 (GPC high) remained comparable, suggesting that the threshold required for anchorage dependent Notch activation in terms of surface anchor antigen expression per cell is low (2,672 surface anchor antigen per cell in SK-PAC31 vs 120,762 surface anchor antigen per cell in SK-PCA60) (FIG. 6C). This suggests that as long as sufficient numbers of anchor antigens are expressed on cells, Notch activation via trans-binding can be achieved (FIG. 8C).

### Method for Antibody Immobilization Assay

For direct immobilization of Notch ligand bi-specific antibodies via absorption, a 96-well plate was first coated with the antibodies at a concentration of 10 micro g/mL for 16 hours at 4° C. After antibody coating, the culture plates were washed cell culture medium before blocking with 5% FBS solution for 2 hours at room temperature. After which, C2C12 Notch reporter cells were seeded at 3×10⁴ cells per well and incubate at 37° C. for 48 hours before performing dual-glo luciferase assay as per manufacturer's protocol (Promega). For non-immobilized condition, antibodies were added right before C2C12 Notch reporter cells were seeded. The luciferase signal was expressed as Relative Luciferase Unit (RLU) and was further normalized to the anti-KLH isotype control's RLU.

### Method for Co-Culture Notch Reporter Assay

Anchor antigen expressing cells (i.e. GPC3/FAP/CD32 overexpressing cells) was seeded at a density of 1×10⁵ cells per well and incubated at 37° C. for 16 hours. Anti-GPC3//Notch ligand-Fc bi-specific antibody or anti-KLH IgG1 isotype control antibody were added at 250 micro g/mL per well and incubated for 1 hour before 3×10⁴ of C2C12 Notch reporter cells were seeded to the wells. After 24 hours of incubation at 37° C., dual-glo luciferase assay was conducted according to manufacturer's protocol (Promega). The luciferase signal was expressed as Relative Luciferase Unit and was further normalized to anti-KLH isotype control's RLU.

### Method for Cell Surface GPC3 Quantification

Cell surface expression of GPC3 was quantified using Quantum Simply Cellular, anti-human kit (Bangs Laboratories) in accordance to manufacturer's recommended protocol. Briefly, 10,000 cells were prepared and stained with an anti-GPC3 antibody for 30 minutes on ice. The 96-well plates were washed twice with HEPES-BSA buffer before the addition of a Goat Anti-Human Kappa PE secondary antibody (Southern Biotech) and incubated on ice for 30 minutes. After washing the plates twice, the samples were analyzed for PE signal with a flow cytometer (BD, Fortessa). A standard curve was generated with microspheres of varying levels of anti-human IgG conjugated to it. Calculation of cell surface GPC3 was done using the QuickCal v2.3 tool provided by manufacturer.

TABLE 4
Table showing sequence ID of molecules
illustrated in FIGS. 7 and 8

| | SEQ ID NO: | | |
|---|---|---|---|
| Molecule name | Chain 1 | Chain 2 | Chain 3 |
| Anti-FAP//Jag1-Fc | 22 | 23 | 3 |
| Anti-KLH//Jag1-Fc | 1 | 2 | 3 |
| Anti-KLH//Jag2-Fc | 1 | 24 | 25 |
| Anti-KLH//DLL1-Fc | 1 | 24 | 26 |
| Anti-KLH//DLL3-Fc | 1 | 24 | 27 |
| Anti-KLH//DLL4-Fc | 1 | 24 | 28 |
| Anti-GPC3//Jag1-Fc | 5 | 6 | 3 |
| Anti-GPC3//Jag2-Fc | 5 | 6 | 25 |
| Anti-GPC3//DLL1-Fc | 5 | 6 | 26 |
| Anti-GPC3//DLL3-Fc | 5 | 6 | 27 |
| Anti-GPC3//DLL4-Fc | 5 | 6 | 28 |
| Anti-KLH/anti-KLH-Fc | 29 | 2 | — |
| Jag1//Jag1-Fc* | 30 | — | — |

TABLE 5
Table showing amino acid sequence of molecules illustrated
FIGS. 7 and 8 and Table 4.

| SEQ ID NO | Amino acid sequence |
|---|---|
| 22 | EVQLLESGGGLVQPGGSLRLSCAASGFTFSSHAMSWVRQAPGKGLE |
| | WVSAIWASGEQYYADSVKGRFTISRDNSKNTLYLQMNSLRAEDTAV |
| | YYCAKGWLGNFDYWGQGTLVTVSSASTKGPSVFPLAPSSKSTSGGT |
| | AALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPEL |
| | RRGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | GLPSSIEKTISKAKGQPREPQVYTLPPSRCEMTKNQVSLSCAVKGFYP |
| | SDIAVEWESNGQPENNYKTTPPVLDSDGSFFLVSKLTVDKSRWQQG |
| | NVFSCSVMHEALHNHYTQKSLSLSP |
| 23 | EIVLTQSPGTLSLSPGERATLSCRASQSVSRSYLAWYQQKPGQAPRLL |
| | IIGASTRATGIPDRFSGSGSGTDFTLTISRLEPEDFAVYYCQQGQVIPPT |
| | FGQGTKVEIKRTVAAPSVFIFPPSDEQLKSGTASVVCLLNNFYPREAK |
| | VQWKVDNALQSGNSQESVTEQDSKDSTYSLSSTLTLSKADYEKHKV |
| | YACEVTHQGLSSPVTKSFNRGEC |
| 24 | DIQMTQSSSSFSVSLGDRVTITCKASEDIYNRLAWYQQKPGNAPRLLI |
| | SGATSLETGVPSRFSGSGSGKDYTLSITSLQTEDVATYYCQQYWSTP |
| | YTFGGGTKLEVKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC |
| 25 | MGYFELQLSALRNVNGELLSGACCDGDGRTTRAGGCGHDECDTYV |
| | RVCLKEYQAKVTPTGPCSYGHGATPVLGGNSFYLPPAGAAGDRARA |
| | RARAGGDQDPGLVVIPFQFAWPRSFTLIVEAWDWDNDTTPNEELLIE |
| | RVSHAGMINPEDRWKSLHFSGHVAHLELQIRVRCDENYYSATCNKF |
| | CRPRNDFFGHYTCDQYGNKACMDGWMGKECKEAVCKQGCNLLHG |
| | GCTVPGECRCSYGWQGRFCDECVPYPGCVHGSCVEPWQCNCETNW |
| | GGLLCDKDLNYCGSHHPCTNGGTCINAEPDQYRCTCPDGYSGRNCE |
| | KAEHACTSNPCANGGSCHEVPSGFECHCPSGWSGPTCALDIDECASN |
| | PCAAGGTCVDQVDGFECICPEQWVGATCQLDANECEGKPCLNAFSC |
| | KNLIGGYYCDCIPGWKGINCHINVNDCRGQCQHGGTCKDLVNGYQC |
| | VCPRGFGGRHCELERDECASSPCHSGGLCEDLADGFHCHCPQGFSGP |
| | LCEVDVDLCEPSPCRNGARCYNLEGDYYCACPDDFGGKNCSVPREP |
| | CPGGACRVIDGCGSDAGPGMPGTAASGVCGPHGRCVSQPGGNFSCI |
| | CDSGFTGTYCHENIDDCLGQPCRNGGTCIDEVDAFRCFCPSGWEGEL |
| | CDTNPNDCLPDPCHSRGRCYDLVNDFYCACDDGWKGKTCHSREFQ |
| | CDAYTCSNGGTCYDSGDTFRCACPPGWKGSTCAVAKNSSCLPNPCV |
| | NGGTCVGSGASFSCICRDGWEGRTCTHNTNDCNPLPCYNGGICVDG |
| | VNWFRCECAPGFAGPDCRINIDECQSSPCAYGATCVDEINGYRCSCP |
| | PGRAGPRCQEVIGFGRSCWSRGTPFPHGSSWVEDCNSCRCLDGRRD |
| | CSKVWCGWKPCLLAGQPEALSAQCPLGQRCLEKAPGQCLRPPCEA |
| | WGECGAEEPPSTPCLPRSGHLDNNCARLTLHFNRDHVPQGTTVGAIC |
| | SGIRSLPATRAVARDRLLVLLCDRASSGASAVEVAVSFSPARDLPDSS |
| | LIQGAAHAIVAAITQRGNSSLLLAVTEVKVETVVTGGSSTEPKSSDKT |
| | HTCPPCPAPELRRGPSVFLFPPKPKDTLMISRTPEVTCVVVDVSHEDP |
| | EVKFNWYVDGVEVHNAKTKPREEQYNSTYRVVSVLTVLHQDWLN |
| | GKEYKCKVSNKGLPSSIEKTISKAKGQPREPQVCTLPPSREEMTKNQ |
| | VSLWCLVKGFYPSDIAVEWESNGQPENNYKTTPPVLDSDGSFFLYSK |
| | LTVDKSRWQQGNVFSCSVMHEALHNRYTQKSLSLSPHHHHHHHH |
| 26 | SGVFELKLQEFVNKKGLLGNRNCCRGGAGPPPCACRTFFRVCLKHY |
| | QASVSPEPPCTYGSAVTPVLGVDSFSLPDGGGADSAFSNPIRFPFGFT |
| | WPGTFSLIIEALHTDSPDDLATENPERLISRLATQRHLTVGEEWSQDL |
| | HSSGRTDLKYSYRFVCDEHYYGEGCSVFCRPRDDAFGHFTCGERGE |
| | KVCNPGWKGPYCTEPICLPGCDEQHGFCDKPGECKCRVGWQGRYC |
| | DECIRYPGCLHGTCQQPWQCNCQEGWGGLFCNQDLNYCTHHKPCK |
| | NGATCTNTGQGSYTCSCRPGYTGATCELGIDECDPSPCKNGGSCTDL |
| | ENSYSCTCPPGFYGKICELSAMTCADGPCFNGGRCSDSPDGGYSCRC |
| | PVGYSGFNCEKKIDYCSSSPCSNGAKCVDLGDAYLCRCQAGFSGRH |
| | CDDNVDDCASSPCANGGTCRDGVNDFSCTCPPGYTGRNCSAPVSRC |
| | EHAPCHNGATCHERGHRYVCECARGYGGPNCQFLLPELPPGPAVVD |
| | LTEKLEGQGGPFPWEPKSSDKTHTCPPCPAPELRRGPSVFLFPPKPKD |
| | TLMISRTPEVTCVVVDVSHEDPEVKFNWYVDGVEVHNAKTKPREEQ |
| | YNSTYRVVSVLTVLHQDWLNGKEYKCKVSNKGLPSSIEKTISKAKG |
| | QPREPQVCTLPPSREEMTKNQVSLWCLVKGFYPSDIAVEWESNGQPE |
| | NNYKTTPPVLDSDGSFFLYSKLTVDKSRWQQGNVFSCSVMHEALHN |
| | RYTQKSLSLSPHHHHHHHH |
| 27 | AGVFELQIHSFGPGPGPGAPRSPCSARLPCRLFFRVCLKPGLSEEAAES |
| | PCALGAALSARGPVYTEQPGAPAPDLPLPDGLLQVPFRDAWPGTFSF |
| | IIETWREELGDQIGGPAWSLLARVAGRRRLAAGGPWARDIQRAGAW |
| | ELRFSYRARCEPPAVGTACTRLCRPRSAPSRCGPGLRPCAPLEDECEA |
| | PLVCRAGCSPEHGFCEQPGECRCLEGWTGPLCTVPVSTSSCLSPRGPS |
| | SATTGCLVPGPGPCDGNPCANGGSCSETPRSFECTCPRGFYGLRCEVS |
| | GVTCADGPCFNGGLCVGGADPDSAYICHCPPGFQGSNCEKRVDRCS |
| | LQPCRNGGLCLDLGHALRCRCRAGFAGPRCEHDLDDCAGRACANG |
| | GTCVEGGGAHRCSCALGFGGRDCRERADPCAARPCAHGGRCYAHF |
| | SGLVCACAPGYMGARCEFPVHPDGASALPAAPPGLRPGDPQRYLEP |
| | KSSDKTHTCPPCPAPELRRGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKGLPSSIEKTISKAKGQPREPQVCTLPPSREE |
| | MTKNQVSLWCLVKGFYPSDIAVEWESNGQPENNYKTTPPVLDSDGS |
| | FFLYSKLTVDKSRWQQGNVFSCSVMHEALHNRYTQKSLSLSPHHHH |
| | HHHH |
| 28 | SGVFQLQLQEFINERGVLASGRPCEPGCRTFFRVCLKHFQAVVSPGPC |
| | TFGTVSTPVLGTNSFAVRDDSSGGGRNPLQLPFNFTWPGTFSLIIEAW |
| | HAPGDDLRPEALPPDALISKIAIQGSLAVGQNWLLDEQTSTLTRLRYS |
| | YRVICSDNYYGDNCSRLCKKRNDHFGHYVCQPDGNLSCLPGWTGE |
| | YCQQPICLSGCHEQNGYCSKPAECLCRPGWQGRLCNECIPHNGCRH |
| | GTCSTPWQCTCDEGWGGLFCDQDLNYCTHHSPCKNGATCSNSGQR |
| | SYTCTCRPGYTGVDCELELSECDSNPCRNGGSCKDQEDGYHCLCPPG |
| | YYGLHCEHSTLSCADSPCFNGGSCRERNQGANYACECPPNFTGSNCE |
| | KKVDRCTSNPCANGGQCLNRGPSRMCRCRPGFTGTYCELHVSDCAR |
| | NPCAHGGTCHDLENGLMCTCPAGESGRRCEVRTSIDACASSPCFNRA |
| | TCYTDLSTDTFVCNCPYGFVGSRCEFPVGLPPSFPWEPKSSDKTHTCP |
| | PCPAPELRRGPSVFLFPPKPKDTLMISRTPEVTCVVVDVSHEDPEVKF |
| | NWYVDGVEVHNAKTKPREEQYNSTYRVVSVLTVLHQDWLNGKEY |
| | KCKVSNKGLPSSIEKTISKAKGQPREPQVCTLPPSREEMTKNQVSLW |
| | CLVKGFYPSDIAVEWESNGQPENNYKTTPPVLDSDGSFFLYSKLTVD |
| | KSRWQQGNVFSCSVMHEALHNRYTQKSLSLSPHHHHHHHH |
| 29 | QVQLQQSGPQLVRPGASVKISCKASGYSFTSYWMHWVNQRPGQGL |
| | EWIGMIDPSYSETRLNQKFKDKATLTVDKSSSTAYMQLSSPTSEDSA |
| | VYYCALYGNYFDYWGQGTTLTVSSASTKGPSVFPLAPSSRSTSESTA |
| | ALGCLVKDYFPEPVTVSWNSGALTSGVHTFPAVLQSSGLYSLSSVVT |
| | VPSSSLGTKTYTCNVDHKPSNTKVDKRVEPKSCDKTHTCPPCPAPEL |
| | RRGPKVFLFPPKPKDTLMISRTPEVTCVVVDVSQEDPEVQFNWYVD |
| | GVEVHNAKTKPREEQFASTYRVVSVLTVLHQDWLNGKEYKCKVSN |
| | KGLPSSIEKTISKAKGQPREPQVYTLPPSREEMTKNQVSLTCL VKGFY |
| | PSDIAVEWESNGQPENNYKTTPPYLDSDGSFFLYSKLTVDKSRWQQG |
| | NVFSCSVMHEALHNHYTQESLSLSPHHHHHHHH |
| 30 | KVCGASGQFELEILSMQNVNGELQNGNCCGGARNPGDRKCTRDEC |
| | DTYFKVCLKEYQSRVTAGGPCSFGSGSTPVIGGNTFNLKASRGNDRN |
| | RIVLPFSFAWPRSYTLLVEAWDSSNDTVQPDSIIEKASHSGMINPSRQ |
| | WQTLKQNTGVAHFEYQIRVTCDDYYYGFGCNKFCRPRDDFFGHYA |
| | CDQNGNKTCMEGWMGPECNRAICRQGCSPKHGSCKLPGDCRCQYG |
| | WQGLYCDKCIPHPGCVHGICNEPWQCLCETNWGGQLCDKDLNYCG |
| | THQPCLNGGTCSNTGPDKYQCSCPEGYSGPNCEIAEHACLSDPCHNR |
| | GSCKETSLGFECECSPGWTGPTCSTNIDDCSPNNCSHGGTCQDLVNG |
| | FKCVCPPQWTGKTCQLDANECEAKPCVNAKSCKNLIASYYCDCLPG |
| | WMGQNCDININDCLGQCQNDASCRDLVNGYRCICPPGYAGDHCER |
| | DIDECASNPCLNGGHCQNEINRFQCLCPTGFSGNLCQLDIDYCEPNPC |
| | QNGAQCYNRASDYFCKCPEDYEGKNCSHLKDHCRTTPCEVIDSCTV |
| | AMASNDTPEGVRYISSNVCGPHGKCKSQSGGKFTCDCNKGFTGTYC |
| | HENINDCESNPCRNGGTCIDGVNSYKCICSDGWEGAYCETNINDCSQ |
| | NPCHNGGTCRDLVNDFYCDCKNGWKGKTCHSRDSQCDEATCNNG |
| | GTCYDEGDAFKCMCPGGWEGTTCNIARNSSCLPNPCHNGGTCVVN |
| | GESFTCVCKEGWEGPICAQNTNDCSPHPCYNSGTCVDGDNWYRCEC |
| | APGFAGPDCRININECQSSPCAFGATCVDEINGYRCVCPPGHSGAKC |
| | QEVSGRPCITMGSVIPDGAKWDDDCNTCQCLNGRIACSKVWCGPRP |
| | CLLHKGHSECPSGQSCIPILDDQCFVHPCTGVGECRSSSLQPVKTKCT |
| | SDSYYQDNCANITFTFNKEMMSPGLTTEHICSELRNLNILKNVSAEYS |
| | IYIACEPSPSANNEIHVAISAEDIRDDGNPIKEITDKIIDLVSKRDGNSSL |
| | IAAVAEVRVQRRPLKNRTDEPKSSDKTHTCPPCPAPEYLGGDSVFLFP |
| | PKPKDVLMISRTPEVTCVVIDVSHEDPEVKFNWYVDGVEVHNAKTK |
| | PREEQYNSTYRVVSVLPVLHQDWLNGKEYKCKVSNKALPKPIEKTIS |
| | KAKGQPREPQVYTLPPSREEMTKNQVSLTCLVKGFYPSDIAVEWESN |
| | GQPENNYKTTPPVLDSDGSFFLYSKLTVDKSRWQQGNVFSCSVMHE |
| | ALHNHYTQKSLSLSP |

### SEQUENCE LISTING

C1-A2019Psq.txt

1. A multispecific antigen-binding molecule comprising:
(i) a first antigen-binding moiety which specifically binds to a Notch receptor on a first target cell, and
(ii) a second antigen-binding moiety which specifically binds to an anchor antigen on a second target cell,
wherein the first target cell and the second target cell are different cells, and
wherein the multispecific antigen-binding molecule activates the Notch signaling pathway in the first target cell when the multispecific antigen-binding molecule is binding to the anchor antigen on the second target cell.

2. The multispecific antigen-binding molecule of claim 1, wherein the first target cell is a tissue stem cell, activated CD4 T-lymphocyte, cell secreting pro-fibrotic factors or pro-tumorigenic cell in tumor microenvironment or non-tumor microenvironment.

3. The multispecific antigen-binding molecule of claim 2, wherein the tissue stem cell is a satellite cell, adult intestinal stem cell or crypt base columnar (CBC) cell.

4. The multispecific antigen-binding molecule of claim 1, wherein the first-binding moiety comprises a Notch-binding domain of a Notch receptor ligand.

5. The multispecific antigen-binding molecule of claim 4, wherein the Notch receptor ligand is a ligand to a Notch1, Notch2, Notch3, or Notch4 receptor.

6. The multispecific antigen-binding molecule of claim 4, wherein the Notch receptor ligand is a Delta protein or Jagged protein.

7. The multispecific antigen-binding molecule of claim 6, wherein the Delta protein is a Delta Like Ligand 1 (DLL1), DLL3, or DLL4.

8. The multispecific antigen-binding molecule of claim 6, wherein the Jagged protein is a Jagged 1 or Jagged 2.

9. The multispecific antigen-binding molecule of claim 1, wherein the first antigen-binding moiety comprises a Fab, scFv, VHH, VL, VH, or single domain antibody that specifically binds to the Notch receptor.

10. The multispecific antigen-binding molecule of claim 1, wherein the second target cell is selected from the group consisting of a muscle cell which is not a satellite cell, activated fibroblast, immune cell that expresses FcgRIIB, GPC3 expressing cancer cell, and cell in the intestinal crypts.

11. The multispecific antigen-binding molecule of claim 10, wherein the immune cell that expresses FcgRIIB is selected from the group consisting of a circulating B lymphocyte, monocyte, neutrophil, lymphoid-dendritic cell and myeloid-dendritic cell.

12. The multispecific antigen-binding molecule of claim 10, wherein the anchor antigen on the second target cell is selected from the group consisting of Calcium Voltage-Gated Channel Subunit Alpha1 S (CACNA1S), Fibroblast activation protein (FAP), Glypican-3 (GPC3) and Fc gamma RIIB (CD32B).

13. The multispecific antigen-binding molecule of claim 1, wherein the second antigen-binding moiety comprises a Fab, scFv, VHH, VL, VH, single domain antibody, ligand, or engineered Fc region that specifically binds to the anchor antigen.

14. The multispecific antigen-binding molecule of claim 1, wherein the multispecific antigen-binding molecule further comprises an Fc region.

15. The multispecific antigen-binding molecule of claim 14, wherein the Fc region is an engineered Fc region which exhibits reduced binding affinity to human Fc gamma receptor, as compared to a native human IgG1 Fc domain.

16. The multispecific antigen-binding molecule of claim 13, wherein the second antigen-binding moiety comprises an engineered Fc region which specifically binds to FcgRIIB.

17. The multispecific antigen-binding molecule of claim 1, wherein the multispecific antigen-binding molecule further comprises one more of the first antigen-binding moiety.

18. The multispecific antigen-binding molecule of claim 1, wherein the multispecific antigen-binding molecule further comprises a third antigen-binding moiety which specifically binds to an anchor antigen on a third target cell.

19. The multispecific antigen-binding molecule of claim 18, wherein the second target cell and the third target cell are different cells or the same cells.

20. A pharmaceutical composition comprising the multispecific antigen-binding molecule of claim 1, and a pharmaceutically acceptable carrier.

21. A method for activating Notch signaling pathway in a first target cell, comprising contacting the first target cell with an effective amount of the multispecific antigen-binding molecule of claim 1.

22. The method of claim 21, wherein the first target cell is in a mammalian subject in vivo.

23. The method of claim 22, wherein the subject is a human.

24. An isolated nucleic acid encoding the multispecific antigen-binding molecule of claim 1.

25. A vector comprising the nucleic acid of claim 24.

26. A host cell comprising the nucleic acid of claim 24.

27. A method of producing the multispecific antigen-binding molecule comprising culturing the host cell of claim 26 under conditions suitable to express the multispecific antigen-binding molecule.

28. A method of treating a Notch receptor-mediated disease or disorder comprising administering an effective amount of the multispecific antigen-binding molecule of claim 1 to a subject.

29. The method of claim 28, wherein the Notch receptor-mediated disease or disorder is selected from a cancer or malignancy; Muscular Dystrophy, Tissue fibrosis; SLE (systemic lupus erythematosus), RA (Rheumatoid arthritis), MS (Multiple sclerosis) or another autoimmune disease.

30. The multispecific antigen-binding molecule of claim 1, wherein each of the first target cell and the second target cell is in a tumor microenvironment or in a non-tumor microenvironment.


SEQUENCE LISTING (32 sequences)

SEQ ID NO: 1 - protein, 444 aa, Artificial Sequence
  an artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRCEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 2 - protein, 214 aa, Artificial Sequence
  an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 1279 aa, Artificial Sequence
  an artificially synthesized sequence
KVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPHHHHHHHH

SEQ ID NO: 4 - protein, 1271 aa, Artificial Sequence
  an artificially synthesized sequence
KVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 5 - protein, 443 aa, Artificial Sequence
  an artificially synthesized sequence
QVQLVQSGAEVKKPGASVKVSCKASGYTFTDYEMHWIRQPPGQGLEWIGAIDGKTPDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRCEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 6 - protein, 219 aa, Artificial Sequence
  an artificially synthesized sequence
DIVMTQSPLSLPVTPGEPASISCRSSQSLVHSNRNTYLQWYQQKPGQAPRLLIYKVSNRFRGVPDRFSGSGSGTDFTLKISRVEAEDVGVYYCIQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 115 aa, Artificial Sequence
  an artificially synthesized sequence
QVQLVQSGAEVKKPGASVKVSCKASGYTFTDYEMHWIRQPPGQGLEWIGAIDGKTPDTAYSQKFKGRVTLTADKSTSTAYMELSSLTSEDTAVYYCTRFYSYTYWGQGTLVTVSS

SEQ ID NO: 8 - protein, 112 aa, Artificial Sequence
  an artificially synthesized sequence
DIVMTQSPLSLPVTPGEPASISCRSSQSLVHSNRNTYLQWYQQKPGQAPRLLIYKVSNRFRGVPDRFSGSGSGTDFTLKISRVEAEDVGVYYCIQNTHVPPTFGQGTKLEIK

SEQ ID NO: 9 - protein, 4 aa, Artificial Sequence
  an artificially synthesized sequence
GGGS

SEQ ID NO: 10 - protein, 4 aa, Artificial Sequence
  an artificially synthesized sequence
SGGG

SEQ ID NO: 11 - protein, 5 aa, Artificial Sequence
  an artificially synthesized sequence
GGGGS

SEQ ID NO: 12 - protein, 5 aa, Artificial Sequence
  an artificially synthesized sequence
SGGGG

SEQ ID NO: 13 - protein, 6 aa, Artificial Sequence
  an artificially synthesized sequence
GGGGGS

SEQ ID NO: 14 - protein, 6 aa, Artificial Sequence
  an artificially synthesized sequence
SGGGGG

SEQ ID NO: 15 - protein, 7 aa, Artificial Sequence
  an artificially synthesized sequence
GGGGGGS

SEQ ID NO: 16 - protein, 7 aa, Artificial Sequence
  an artificially synthesized sequence
SGGGGGG

SEQ ID NO: 17 - protein, 19 aa, Artificial Sequence
  an artificially synthesized sequence
MGWSCIILFLVATATGVHS

SEQ ID NO: 18 - protein, 330 aa, Homo sapiens [sequence omitted]

SEQ ID NO: 19 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 444 aa, Artificial sequence
  an artificially synthesised sequence
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRCEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 23 - protein, 215 aa, Artificial sequence
  an artificially synthesised sequence
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, Artificial sequence; identical to SEQ ID NO: 2
  an artificially synthesised sequence

SEQ ID NO: 25 - protein, 1292 aa, Artificial sequence
  an artificially synthesised sequence
MGYFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCEKAEHACTSNPCANGGSCHEVPSGFECHCPSGWSGPTCALDIDECASNPCAAGGTCVDQVDGFECICPEQWVGATCQLDANECEGKPCLNAFSCKNLIGGYYCDCIPGWKGINCHINVNDCRGQCQHGGTCKDLVNGYQCVCPRGFGGRHCELERDECASSPCHSGGLCEDLADGFHCHCPQGFSGPLCEVDVDLCEPSPCRNGARCYNLEGDYYCACPDDFGGKNCSVPREPCPGGACRVIDGCGSDAGPGMPGTAASGVCGPHGRCVSQPGGNFSCICDSGFTGTYCHENIDDCLGQPCRNGGTCIDEVDAFRCFCPSGWEGELCDTNPNDCLPDPCHSRGRCYDLVNDFYCACDDGWKGKTCHSREFQCDAYTCSNGGTCYDSGDTFRCACPPGWKGSTCAVAKNSSCLPNPCVNGGTCVGSGASFSCICRDGWEGRTCTHNTNDCNPLPCYNGGICVDGVNWFRCECAPGFAGPDCRINIDECQSSPCAYGATCVDEINGYRCSCPPGRAGPRCQEVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPHHHHHHHH

SEQ ID NO: 26 - protein, 762 aa, Artificial sequence
  an artificially synthesised sequence
SGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPHHHHHHHH

SEQ ID NO: 27 - protein, 704 aa, Artificial sequence
  an artificially synthesised sequence
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPHHHHHHHH

SEQ ID NO: 28 - protein, 741 aa, Artificial sequence
  an artificially synthesised sequence
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPHHHHHHHH

SEQ ID NO: 29 - protein, 452 aa, Artificial sequence
  an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELRRGPKVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSPHHHHHHHH

SEQ ID NO: 30 - protein, 1271 aa, Artificial sequence
  an artificially synthesised sequence
KVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDEPKSSDKTHTCPPCPAPEYLGGDSVFLFPPKPKDVLMISRTPEVTCVVIDVSHEDPEVKFNWYVDGVEVHNAKTKPREEQYNSTYRVVSVLPVLHQDWLNGKEYKCKVSNKALPKPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 31 - protein, 116 aa, Artificial sequence
  an artificially synthesised sequence
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 32 - protein, 108 aa, Artificial sequence
  an artificially synthesised sequence
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK